(19) United States
(12) Patent Application Publication
(10) Pub. No.: US 2023/0093147 A1
(43) Pub. Date: Mar. 23, 2023

(54) METHODS AND COMPOSITIONS RELATING TO IMPROVED COMBINATION THERAPIES

(71) Applicant: PRESIDENT AND FELLOWS OF HARVARD COLLEGE, Cambridge, MA (US)

(72) Inventors: Samir MITRAGOTRI, Cambridge, MA (US); Debra WU, Cambridge, MA (US)

(73) Assignee: PRESIDENT AND FELLOWS OF HARVARD COLLEGE, Cambridge, MA (US)

(21) Appl. No.: 17/801,872

(22) PCT Filed: Mar. 2, 2021
(86) PCT No.: PCT/US2021/020401

§ 371 Date: Aug. 24, 2022

Related U.S. Application Data
(60) Provisional application No. 62/986,967, filed on Mar. 9, 2020.

(51) Int. Cl. A61K 31/7068; A61K 9/127; A61K 31/704; A61K 31/7028; A61P 35/00

(52) U.S. Cl. CPC A61K 31/7068; A61K 9/127; A61K 31/704; A61K 31/7028; A61P 35/00

(57) ABSTRACT

The technology described herein is directed to in vitro methods of identifying or selecting combinations of therapeutic agents that are effective in vivo. These methods provide improved methods of treatment, e.g., treatment of cancer. Further, provided herein are novel combinations of anti-cancer agents which are demonstrated to have surprising efficacy.

### CROSS-REFERENCE TO RELATED APPLICATIONS

This application claims benefit under 35 U.S.C. § 119(e) of U.S. Provisional Application No. 62/986,967 filed Mar. 9, 2020, the contents of which are incorporated herein by reference in their entirety.

### TECHNICAL FIELD

The technology described herein relates to methods and compositions for improved combination therapies.

### BACKGROUND

Combination therapies are a primary avenue for improving clinical outcomes. However, current methods of finding optimal or even functional combination therapies involves screening in vitro for maximal effects or IC₅₀. When the lead combination is then tested in vivo, the combination rarely displays the promising results suggested by the in vitro test. Improved in vitro assays for identifying clinically-relevant combination therapies are needed.

### SUMMARY

As described herein, the inventors have discovered that the Hill coefficient obtained from in vitro tests of combination therapies successfully predicts the in vitro performance of those same combinations. The Hill coefficient does not measure the total effect obtained from the combination, but rather the steepness of the dose-response curve.

In one aspect of any of the embodiments, provided herein is a method of treating cancer in a subject in need thereof with a drug combination, the method comprising administering to the subject a drug combination having an in vitro dose response Hill coefficient greater than 0.8.

In one aspect of any of the embodiments, provided herein is a method of treating cancer in a subject in need thereof with a drug combination, the method comprising:
a. contacting cancer cells in vitro with at least two different candidate combinations of the candidate drugs;
b. measuring the in vitro dose response of the cancer cells to each candidate combination of step a;
c. calculating the Hill coefficient from the dose response measured in step b;
d. administering the combination with the largest Hill coefficient to the subject.

In one aspect of any of the embodiments, provided herein is a method of treating cancer in a subject in need thereof with a drug combination, the method comprising administering to the subject a drug combination determined to have the largest in vitro dose response Hill coefficient from among a group of different drug combinations.

In one aspect of any of the embodiments, provided herein is a method of selecting the most therapeutically effective combination of anti-cancer drugs from a pool of candidate drugs, the method comprising:
a. contacting cancer cells in vitro with at least two different candidate combinations of the candidate drugs;
b. measuring the in vitro dose response of the cancer cells to each candidate combination of step a;
c. calculating the Hill coefficient from the dose response measured in step b;
d. selecting the combination with the largest Hill coefficient as the most therapeutically effective of the candidate combinations.

In one aspect of any of the embodiments, provided herein is a method of manufacturing a therapeutically effective combination of anti-cancer drugs from a pool of candidate drugs, the method comprising:
a. forming at least two different candidate combinations of candidate drugs from a pool of candidate drugs;
b. contacting cancer cells in vitro with the at least two different candidate combinations of the candidate drugs;
c. measuring the in vitro dose response of the cancer cells to each candidate combination in step b;
d. calculating the Hill coefficient from the dose response measured in step c;
e. selecting the combination with the largest Hill coefficient as the most therapeutically effective of the candidate combinations; and
f. providing the combination selected in step e as the therapeutically effective combination of anti-cancer drugs.

In some embodiments of any of the aspects, the largest Hill coefficient is greater than 0.8. In some embodiments of any of the aspects, the largest Hill coefficient is greater than 1.0. In some embodiments of any of the aspects, the largest Hill coefficient is greater than 1.5.

In some embodiments of any of the aspects, the cancer cells are primary cancer cells obtained from a/the subject. In some embodiments of any of the aspects, the cancer cells are primary cancer cells obtained from a/the subject during treatment or diagnosis. In some embodiments of any of the aspects, the cancer cells are primary cancer cells obtained from a/the subject no more than 3 months prior to the determination of the Hill coefficients.

In some embodiments of any of the aspects, the combination or candidate combination is a pairwise combination. In some embodiments of any of the aspects, the combination or candidate combination is a combination of three, four, or more drugs or candidate drugs. In some embodiments of any of the aspects, the drug combination or candidate combination is a. Doxorubicin and b. at least one of 5-fluorouracil, gemcitabine, and irinotecan. In some embodiments of any of the aspects, the drug combination or candidate combination is at least two of: doxil; doxorubicin; 5-fluorouracil; gemcitabine; irinotecan; vincristine; mifamurtide; cytarbine; and daunarubicin.

In some embodiments of any of the aspects, the drug combination or candidate combination is provided in a liposome, wherein each member of the combination is present in the liposome. In some embodiments of any of the aspects, the drug combination or candidate combination is provided in a mixture of liposomes, wherein each liposome comprises only one member of the combination. In some embodiments of any of the aspects, combinations or candidate combinations differ from other combinations or candidate combinations in the identity of the drugs therein, the relative dose of the drugs therein, and/or the liposome formulation.

In one aspect of any of the embodiments, provided herein is a method of treating cancer in a subject in need thereof, the method comprising administering to the subject a liposomal composition comprising individual liposomes each comprising both gemcitabine and doxorubicin. In one aspect of any of the embodiments, provided herein is a liposomal composition comprising individual liposomes each comprising both gemcitabine and doxorubicin. In some embodiments of any of the aspects, the liposomal composition is for use in a method of treating cancer. In some embodiments of any of the aspects, the gemcitabine and doxorubicin are present at a molar ratio of from 0.5:1 to 2:1. In some embodiments of any of the aspects, the gemcitabine and doxorubicin are present at a molar ratio of about 1:1.

### BRIEF DESCRIPTION OF THE DRAWINGS

FIGS. 1A-1E depict release profiles of all liposomal encapsulations. All release samples were analyzed for drug concentration by HPLC (n=3, error bars=SD). (FIG. 1A) DOX-L (FIG. 1B) 5FURW/DOX-L (FIG. 1C) 5FURW/DOX-LR=2.5 (FIG. 1D) IRIN/DOX-L (FIG. 1E) GEM/DOX-L.

FIGS. 2A-2F depict pharmacokinetic release profiles demonstrating that liposomes cause the drug ratios to remain relatively conserved over the 24 hour period (n=3). (FIG. 2A) DOX-L (FIG. 2B) 5FURW/DOX-L (FIG. 2C) 5FURW/DOX-L_{R}=_{2.5} (FIG. 2D) IRIN/DOX-L (FIG. 2E) GEM/DOX-L (FIG. 2F) Drug ratio of each drug combination.

FIGS. 3A-3D demonstrate that cellular inhibition was compared between free drugs, free drug combinations, and co-encapsulated liposomal formulations. (FIGS. 3A-3C) represent the dose-response Hill equation fits (n=12) for the free drug treatments, and (FIG. 3D) represents the dose-response fits for the liposomal formulations. (FIG. 3A) DOX, 5FURW, and 5FURW/DOX in R=1 and R-2.5 molar ratios (FIG. 3B) DOX, IRIN, and R=1 molar ratio of IRIN/DOX (FIG. 3C) DOX, GEM, and R=1 molar ratio of GEM/DOX (FIG. 3D) Dose-response of liposomal formulations with equimolar ratios unless specified otherwise.

FIGS. 4A-4F demonstrate that a study of in vivo performance of the liposomal formulations was initiated with 4T1 murine breast cancer cells subcutaneously injected above the 4th abdominal mammary fat pad. (FIG. 4A) An untreated control group was compared to liposomal formulations containing DOX, 5FURW/DOX at molar ratios R=1 and R=2.5, IRIN/DOX at R=1, and GEM/DOX at R=1. (FIG. 4B) Mice mass was used to reflect the general wellbeing of the animals. No mouse lost more than 15% body weight during the duration of this study. (FIG. 4C) Tumors were excised and weighed at the end of 10 days after administration of the liposomal formulations. (FIG. 4D) Tumor mass did not correlate with IC₅₀ (R²=0.16). (FIG. 4E) A strong negative correlation between HC of liposomal dose-response curve and tumor mass was identified (R²=0.92). (FIG. 4F) No correlation between CI and tumor mass was found (R²=5.5×10⁻⁶).

FIG. 5 depicts tumor-associated immune cell phenotyping. Cells were identified by characteristic markers (FIG. 11). After treatment of 4T1 murine tumors with the liposomal formulations containing DOX, GEM/DOX, 5FURW/DOX, and IRIN/DOX, formulations with better in vivo tumor response had higher amounts of anti-tumor (M1) macrophages and lower Gr-1/Ly6G+neutrophils. GEM/DOX and IRIN/DOX had significantly lower amounts of M2 alternatively activated macrophages, which are commonly associated with poor tumor prognosis. No significant changes in the proportion of cells associated with an adaptive immune response was observed, although CD4+ and CD8+ T cells were slightly elevated across all liposomal formulation treatments.

FIGS. 6A-6B depict a survival study of GEM/DOX-L, GEM-L, and DOX-L liposomes. GEM/DOX-L was dosed at 3 mg/kg DOX and 1.55 mg/kg GEM (n=7). DOX-L was dosed at 6 mg/kg DOX (n=7) and GEM-L was dosed at 3.1 mg/kg GEM (n=8). Intravenous injections were administered every third day for a total of four injections (indicated by black arrows). (FIG. 6A) Tumor volumes showed controlled tumor growth with no significance between treatment groups; however, control tumors reached 1000 mm³ by Day 26. The volume measurements were made for surviving mice (DOX: n=6, GEM: n=3, and GEM/DOX: n=6). (FIG. 6B) Kaplan Meier survival graph shows significant differences in survival profiles (Table 7 and 8, p<0.05).

FIG. 7 demonstrates that tumor mass shows an inverse correlation with increasing Hill coefficient of the combined free drugs. All drugs were combined in equimolar ratios unless specified otherwise.

FIG. 8 demonstrates that dendritic cells and neutrophils were analyzed in the tumor immune infiltrate after tumor extraction and digestion. There was no significant enhancement in proliferation after treatment with liposomal formulations. Refer to FIG. 11 for identification of cell phenotypes.

FIGS. 9A-9F depict the M1 (CD80+) to M2 (CD206+) ratio of all groups. There was an observed inverse correlation between tumor size and M1/M2 ratio for IRIN/DOX-L and GEM/DOX-L. 5FURW/DOX-L and DOX-L also displayed a slight inverse correlation. Other treatment groups displayed no specific pattern. (FIG. 9A) Control (FIG. 9B) DOX-L (FIG. 9C) 5FURW/DOX-L (FIG. 9D) 5FURW/DOX-L_{R=2.5} (FIG. 9E) IRIN/DOX-L (FIG. 9F) GEM/DOX-L.

FIGS. 10A-10B. FIG. 10A demonstrates that there was no weight loss above 15% in the body weight of mice in the DOX-L, GEM/DOX-L, and control group. (FIG. 10B) GEM-L proved to be toxic during treatment (four injections, given every third day), with five out of eight mice in the treatment group losing more than 15% body weight before the last injection was given. Each curve indicates one mouse.

FIG. 11 depicts the immune phenotyping schematic used to identify immune system cells.

FIG. 12 depicts an exemplary embodiment of the systematic design of chemotherapeutic drug combinations.

FIGS. 13A-13F depict in vitro activation of JAWSII cells alone and in co-culture with 4T1 cells. Experiments were conducted in triplicate wells, and quantification is displayed as fold increases in mean fluorescence intensity compared to untreated control JAWSII cells (FIGS. 13C-13D) or equivalent blank liposome treatment in the 4T1 and JAWSII co-culture (FIGS. 13E-13F). (FIG. 13A) Representative shift of JAWSII cells treated with blank liposomes (B-L), MPLA liposomes (MPLA-L) and LPS. (FIG. 13B) Representative shift of JAWSII cells in co-culture with 4T1 cells, after treatment with MPLA-L, DOX-L, and DOX/MPLA-L. (FIG. 13C) MHCII expression in JAWSII cells. (FIG. 13D) CD86 expression in JAWSII cells. (FIG. 13E) MHCII expression in 1:1 4T1:JAWSII co-culture. (FIG. 13F) CD86 expression in 1:1 4T1:JAWSII co-culture.

FIGS. 14A-14B depict GEM/DOX-L and GEM/DOX/MPLA-L in vitro toxicity on 4T1 cells. Both treatments displayed similar dose-response behavior on two different seeding densities. Error bars represent standard deviation with n=6. (FIG. 14A) 500 4T1 cells/well. (FIG. 14B) 5000 4T1 cells/well.

FIGS. 15A-15B depict release profile of liposomal formulations over 24 h at 37° C. in PBS. Error bars represent n=5, and statistical significance between groups was measured using Student's t test. (FIG. 15A) GEM release. (FIG. 15B) DOX release.

FIGS. 16A-16D depict immune profiling of 4T1 tumors showed increased dendritic cell activation. Expression levels are shown using mean fluorescent intensity. (FIG. 16A) MHC I expression. (FIG. 16B) MHC II expression. (FIG. 16C) MHC I/MHC II ratio. (FIG. 16D) CD86 expression.

FIGS. 17A-17C depict the macrophage population within 4T1 tumors as determined by flow cytometry immune profiling. (FIG. 17A) CD80+F4/80+M1 macrophages. (FIG. 17B) CD206+F4/80+M2 macrophages. (FIG. 17C) M1/M2 ratio.

FIGS. 18A-18C depict treatment efficacy of GEM/DOX/MPLA-L and GEM/DOX-L in an orthotopic 4T1 tumor model. Three injections of 100 μl of 0.54 mg/ml DOX and 0.28 mg/ml GEM were injected, which translates to 3 mg/kg DOX and 1.55 mg/ml GEM. Mice treated with GEM/DOX/MPLA-L received 5.7 μg MPLA per injection. (FIG. 18A) Tumor volume measurements, with control (n=8), GEM/DOX-L (n=8), and GEM/DOX/MPLA-L (n=5). Difference in n arises from fully regressed tumors, which were removed from the tumor volume measurements. Significance is displayed for the final tumor size of both treatment groups in comparison to the control group. (FIG. 18B) Mice weight measurements for GEM/DOX-L, GEM/DOX/MPLA-L, and control group. Reported significance is for the GEM/DOX/MPLA-L group relative to the control group. (FIG. 18C) Tumor mass after extraction on day 27 of study, with control (n=6, due to prior euthanasia of two mice), GEM/DOX-L (n=8), and GEM/DOX/MPLA-L (n=2) due to absence of tumors.

FIGS. 19A-19C demonstrate that GEM/DOX-L and GEM/DOX/MPLA-L were compared in terms of efficacy in a tumor rechallenge study. Two injections of 100 μl of 0.54 mg/ml DOX and 0.28 mg/ml GEM were injected, which translates to 3 mg/kg DOX and 1.55 mg/ml GEM. Mice treated with GEM/DOX/MPLA-L received 4.3 μg MPLA per injection. (FIG. 19A) Tumor volume was recorded after two injections of DOX-L and free GEM, GEM/DOX-L, and GEM/DOX/MPLA-L with equivalent doses of 3 mg/kg DOX and 1.55 mg/kg GEM. Significance is reported in terms of comparing control group to DOX-L and free GEM, as well as DOX-L and free GEM to GEM/DOX-L. (FIG. 19B) Upon tumor rechallenge of the GEM/DOX-L and GEM/DOX/MPLA-L treatment groups, little difference was observed in tumor volume, and the previously measured control group. (FIG. 19C) Mice weight remained consistent throughout the study

FIGS. 20A-20B. (FIG. 20A) The mean fluorescence intensity fold increase of calreticulin on 4T1 cells in response to DOX (10 μM) treatment both alone and in combination with blank liposomes and MPLA-L (5 μg) (n=3). Experiment was performed in triplicate wells. Combination with liposomes significantly increased calreticulin exposure. The fold increase of calreticulin is in reference to untreated 4T1 cells. (FIG. 20B) Co-culture of 1:1 4T1 and JAWSII cells significantly upregulated co-stimulatory ligand CD40 when treated DOX/MPLA-L (n=3). The fold increase of CD40 is in reference to treatment with an equivalent amount of blank liposomes.

FIG. 21 depicts representative flow gating strategy for in vitro experiments involving 4T1 cells.

FIG. 22 depicts representative gating and analysis of dendritic cells in co-culture with 4T1 cells.

FIGS. 23A-23B depict the release profile of liposomal formulations over 24 hours at 37° C. in PBS. Error bars represent n=5. (FIG. 23A) GEM/DOX-L (FIG. 23B) GEM/DOX/MPLA-L.

FIGS. 24A-24B depict immune profiling of 4T1 tumors reveals negligible differences in GEM/DOX-L and GEM/DOX/MPLA-L in regards to (FIG. 24A) CD11c+CD11b+ dendritic cells and (FIG. 24B) Ly6G+CD11b+ MDSCs.

FIG. 25 depicts representative gating of dendritic cells and macrophages after tumor extraction and fluorescent antibody staining. Subsequent numbering indicate gates with the previous number as the parent gate.

FIGS. 26A-26B depict dendritic cell and macrophage population shown as a percentage of total measured cells. (FIG. 26A) CD11b+CD11c+ dendritic cells (FIG. 26B) F4/80+ macrophages.

FIG. 27 depicts representative gating of dendritic cells and Ly6G+ myeloid-derived suppressor cells. Subsequent numbering indicate gates with the previous number as the parent gate.

FIG. 28 depicts a graph of the mass of 4T1 tumors prior to tumor dissociation for immune profiling.

FIG. 29 depicts that tumors after extraction on day 27 of the efficacy study.

FIG. 30 depicts a graph of tumor mass comparison between GEM/DOX-L (n=8) and GEM/DOX/MPLA-L (n=2) after tumor extraction.

FIG. 31. Top panel presents polymer drug conjugate tumor growth inhibition data published in J Control Release. 2017 Dec. 10; 267:191-202; which is incorporated by reference herein in its entirety. The bottom panel (also provide herein as FIG. 6A) presents liposome drug formulation tumor growth inhibition data. Both graphs depict tumor growth of the 4T1 Tumor model. “L” refers to a liposome formulation, prepared according to Example 2. GEM/DOX-L was dosed at 3 mg/kg DOX and 1.55 mg/kg GEM.

### DETAILED DESCRIPTION

Finding drug combinations that work effectively in vivo has proved challenging and unpredictable. Traditional approaches that measure in vitro synergy do not predictably carry those advantages into in vivo use. As described herein, the inventors have found that effective in vivo combinations can be identified in vitro by measuring the in vitro dose-response Hill coefficient. The higher the in vitro dose-response Hill coefficient, the greater effectiveness of the combination when used in vivo.

Accordingly, providing herein are methods of selecting or identifying drug combinations suitable for use in vivo by measuring the in vitro dose response Hill coefficient for each of the one or more different combinations and selecting or identifying the combination with the highest in vitro dose response Hill coefficient for use in vivo, or as the combination likely to be or most likely to be effective in vivo. In one aspect of any of the embodiments, described herein is a method of selecting the most therapeutically effective combination of drugs from a pool of candidate drugs, the method comprising:
a. contacting cells in vitro with at least two different candidate combinations of the candidate drugs;
b. measuring the in vitro dose response of the cells to each candidate combination of step a;
c. calculating the Hill coefficient from the dose response measured in step b;
d. selecting the combination with the largest Hill coefficient as the most therapeutically effective of the candidate combinations.
   In another aspect of any of the embodiments, described herein is a method of manufacturing a therapeutically effective combination of drugs from a pool of candidate drugs, the method comprising:
a. forming at least two different candidate combinations of candidate drugs from a pool of candidate drugs;
b. contacting cells in vitro with the at least two different candidate combinations of the candidate drugs;
c. measuring the in vitro dose response of the cells to each candidate combination in step b;
d. calculating the Hill coefficient from the dose response measured in step c;
e. selecting the combination with the largest Hill coefficient as the most therapeutically effective of the candidate combinations; and
f. providing or manufacturing the combination selected in step e as the therapeutically effective combination of drugs.

The response of the cells can be any measureable or observable response which is therapeutically relevant. For example, if the in vivo use is as a chemotherapeutic or cancer therapy, the response of the cells can be changes in cell death, cell cycle arrest, cell growth, cell proliferation, or the like. As a further example, if the in vivo use is as an anti-inflammatory, the response of the cells can be changes in cytokine production. If the in vivo use is as a cholesterol treatment, the response of the cells can be, e.g., changes in cholesterol metabolism or catabolism. If the in vivo use is as a diabetes treatment, the response of the cells can be, e.g., changes in insulin production or insulin sensitivity. The responses can be detected by microscopy, bioassay, or any other method known in the art. One of skill in the art can readily identify a suitable response and bioassay for any given in vivo therapeutic use. The cells themselves can be, e.g., diseased cells, cells which model a disease, or cells which are intended to be targeted by the drug combination for a therapeutic purpose (e.g., healthy immune cells if the drug combination is intended for use as an immune stimulating treatment for patients with cancer).

In some embodiments of any of the aspects, a Hill coefficient can be measured by determining the survival and/or proliferation rates of cells. In some embodiments of any of the aspects, a Hill coefficient can be measured by determining the level of an activity or marker in a cell, e.g., immune cell activity, or levels of a cancer biomarker. Assays for the foregoing are well known in the art and can include methods to measure gene expression products, e.g., protein level (such as ELISA (enzyme linked immunosorbent assay), lateral flow immunoassay (LFIA), western blot, immunoprecipitation, immunohistochemistry, immunocytochemistry, immunofluorescence using detection reagents such as an antibody or protein binding agents, radioimmunological assay (RIA); sandwich assay; fluorescence in situ hybridization (FISH); immunohistological staining; radioimmunometric assay; immunofluoresence assay; mass spectroscopy and/or immunoelectrophoresis assay), or nucleic acid level (such as PCR procedures, RT-PCR, quantitative RT-PCR Northern blot analysis, differential gene expression, RNAse protection assay, microarray based analysis, next-generation sequencing; hybridization methods, etc.)

In some embodiments of any of the aspects, the in vivo use is cancer, e.g., the drugs are anti-cancer drugs and/or anti-cancer candidate drugs. In some embodiments of any of the aspects, the cells can be cancer cells, e.g., cancer cell lines, primary cancer cells, or the like.

In one aspect of any of the embodiments, described herein is a method of selecting the most therapeutically effective combination of anti-cancer drugs from a pool of candidate anti-cancer drugs, the method comprising:
a. contacting cancer cells in vitro with at least two different candidate combinations of the candidate anti-cancer drugs;
b. measuring the in vitro dose response of the cancer cells to each candidate combination of step a;
c. calculating the Hill coefficient from the dose response measured in step b;
d. selecting the combination with the largest Hill coefficient as the most therapeutically effective of the candidate combinations.
   In another aspect of any of the embodiments, described herein is a method of manufacturing a therapeutically effective combination of anti-cancer drugs from a pool of candidate anti-cancer drugs, the method comprising:
a. forming at least two different candidate combinations of candidate anti-cancer drugs from a pool of anti-cancer candidate drugs;
b. contacting cancer cells in vitro with the at least two different candidate combinations of the candidate anti-cancer drugs;
c. measuring the in vitro dose response of the cancer cells to each candidate combination in step b;
d. calculating the Hill coefficient from the dose response measured in step c;
e. selecting the combination with the largest Hill coefficient as the most therapeutically effective of the candidate combinations; and
f. providing or manufacturing the combination selected in step e as the therapeutically effective combination of anti-cancer drugs.

In some embodiments of any of the aspects, multiple effective combinations can be selected as alternatives and/or a combination must exceed a threshold to be selected. In some embodiments of any of the aspects, a drug combination must have an in vitro dose response Hill coefficient greater than 0.8 to be selected, provided, or administered. In some embodiments of any of the aspects, a drug combination must have an in vitro dose response Hill coefficient greater than 0.9 to be selected, provided, or administered. In some embodiments of any of the aspects, a drug combination must have an in vitro dose response Hill coefficient greater than 1.0 to be selected, provided, or administered. In some embodiments of any of the aspects, a drug combination must have an in vitro dose response Hill coefficient greater than 1.1 to be selected, provided, or administered. In some embodiments of any of the aspects, a drug combination must have an in vitro dose response Hill coefficient greater than 1.2 to be selected, provided, or administered. In some embodiments of any of the aspects, a drug combination must have an in vitro dose response Hill coefficient greater than 1.3 to be selected, provided, or administered. In some embodiments of any of the aspects, a drug combination must have an in vitro dose response Hill coefficient greater than 1.4 to be selected, provided, or administered. In some embodiments of any of the aspects, a drug combination must have an in vitro dose response Hill coefficient greater than 1.5 to be selected, provided, or administered. In some embodiments of any of the aspects, a drug combination must have an in vitro dose response Hill coefficient greater than 1.6 to be selected, provided, or administered.

In one aspect of any of the embodiments, described herein is a method of treating a subject with a drug combination, the method comprising:
a. contacting cells in vitro with at least two different candidate combinations of candidate drugs;
b. measuring the in vitro dose response of the cells to each candidate combination of step a;
c. calculating the Hill coefficient from the dose response measured in step b;
d. administering the combination with the largest Hill coefficient to the subject.
   In one aspect of any of the embodiments, described herein is a method of treating a subject with a drug combination, the method comprising administering to the subject a drug combination determined to have the largest in vitro dose response Hill coefficient from among a group of different drug combinations.

In one aspect of any of the embodiments, described herein is a method of treating cancer in a subject in need thereof with a drug combination, the method comprising:
a. contacting cancer cells in vitro with at least two different candidate combinations of candidate drugs;
b. measuring the in vitro dose response of the cancer cells to each candidate combination of step a;
c. calculating the Hill coefficient from the dose response measured in step b;
d. administering the combination with the largest Hill coefficient to the subject.
   In one aspect of any of the embodiments, described herein is a method of treating cancer in a subject in need thereof with a drug combination, the method comprising administering to the subject a drug combination determined to have the largest in vitro dose response Hill coefficient from among a group of different drug combinations.

In some embodiments of any of the aspects, the cells used in vitro in the methods described herein can be cancer cells. In some embodiments of any of the aspects, the cancer cells are cells from a cancer cell line or are primary cancer cells. In some embodiments of any the aspects, the cells are obtained from a subject, e.g., during a treatment, diagnosis, and/or biopsy. In some embodiments of any the aspects, the cells are obtained from the subject (e.g, the subject to be administered the combination), e.g., during a treatment, diagnosis, and/or biopsy. Such embodiments permit identifying treatments and combinations that are particularly effective for the individual patient. In some embodiments of any of the aspects, the cells are obtained from the subject no more than 3 months prior to the determination of the Hill coefficient (e.g., no more than 3 months prior to the contacting step). In some embodiments of any the aspects, the cells are obtained from the subject no more than 2 months prior to the determination of the Hill coefficient (e.g., no more than 2 months prior to the contacting step). In some embodiments of any of the aspects, the cells are obtained from the subject no more than 1 month prior to the determination of the Hill coefficient (e.g., no more than 1 month prior to the contacting step). In some embodiments of any of the aspects, the cells are obtained from the subject no more than 3 weeks prior to the determination of the Hill coefficient (e.g., no more than 3 weeks prior to the contacting step). In some embodiments of any of the aspects, the cells are obtained from the subject no more than 2 weeks prior to the determination of the Hill coefficient (e.g., no more than 2 weeks prior to the contacting step). In some embodiments of any of the aspects, the cells are obtained from the subject no more than 1 week prior to the determination of the Hill coefficient (e.g., no more than 1 week prior to the contacting step). In some embodiments of any of the aspects, the cells are obtained from the subject no more than 1 day prior to the determination of the Hill coefficient (e.g., no more than 1 day prior to the contacting step).

The cells can be individual cells, or part of a culture, monolayer, multilayer, organoid, tissue, or the like during the contacting step. Alternatively, the cells can be in an organ-on-a-chip device during the contacting step.

In some embodiments of any of the aspects, the cells can be in a sample or isolated from a sample. The term “sample” or “test sample” as used herein denotes a sample taken or isolated from a biological organism, e.g., a blood or plasma sample from a subject. In some embodiments of any of the aspects, the subject is the same subject to be treated, e.g., to be administered the combination of drugs. In some embodiments of any of the aspects, the present invention encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. In some embodiments of any of the aspects, the subject can be a human subject. In some embodiments of any of the aspects, the sample obtained from a subject can be a biopsy sample. In some embodiments of any of the aspects, the sample obtained from a subject can be a blood or serum sample.

The term “test sample” also includes untreated or pretreated (or pre-processed) biological samples. In some embodiments of any of the aspects, a test sample can comprise cells from a subject. The test sample can be obtained by removing a sample from a subject, but can also be accomplished by using a previously isolated sample (e.g. isolated at a prior timepoint and isolated by the same or another person). In some embodiments of any of the aspects, the test sample can be an untreated test sample. As used herein, the phrase “untreated test sample” refers to a test sample that has not had any prior sample pre-treatment except for dilution and/or suspension in a solution.

As used herein “combination” refers to a group of two or more substances for use together, e.g., for administration to the same subject. The two or more substances can be present in the same formulation in any molecular or physical arrangement, e.g, in an admixture, in a solution, in a mixture, in a suspension, in a colloid, in an emulsion. The formulation can be a homogeneous or heterogenous mixture. Alternatively, the two or more substances can be present in two or more separate formulations, e.g., in a kit or package comprising multiple formulations in separate containers, to be administered to the same subject or added to the same cell/culture. In some embodiments of any of the aspects, the two or more substances active compound(s) can be comprised by the same or different superstructures, e.g., nanoparticles, liposomes, vectors, cells, scaffolds, or the like, and said superstructure is in solution, mixture, admixture, suspension with a solvent, carrier, or some of the two or more substances.

A combination can be defined by the identity of the elements/members and in some embodiments, the relative amounts of the elements/members. In some embodiments of any of the aspects, a combination is a group of specific elements/members at any relative amount. In some embodiments of any of the aspects, a combination is a group of specific elements/members, at a specified relative amount. Thus, different combinations might differ in their constituent members, or they might have the same constituent members but differ in the relative amounts of those members.

A combination or candidate combination can be a pairwise, three-way, four-way, or greater complexity combination of drugs or candidate drugs. A step of administering or contacting with a combination can comprise providing the combination's elements in a single composition/formulation, or administering/contacting with each element separately such that all elements are eventually present in the same subject or in contact with the same cell (e.g., in the cell's culture medium). Alternatively, the elements of the combination can be provided in a single composition/formulation (e.g., mixture, solution, emulsion, etc.) such that all elements of the combination can be administered or contacted within a single step.

In some embodiments of any of the aspects, a combination is provided in a liposome, wherein each member/element of the combination is present in the liposome. In some embodiments, of any of the aspects, the drug combination or candidate combination is provided in a mixture of liposomes, wherein each liposome comprises only one member/element of the combination but the mixture comprises all members/elements of the combination.

In some embodiments of any of the aspects, a combination's identity can further involve the liposome formulation. Accordingly, a combination can involve a specific type, size, or concentration of liposomes, and/or a specific drug distribution in the liposomes. Drug distribution can refer to where, in or on, the liposome the drug is found and/or whether each drug is found on each liposome vs. whether different drugs are found on different liposomes. Thus, different combinations might:
i) differ in their constituent members,
ii) have the same constituent members but differ in the relative amounts of those members,
iii) differ in the liposome formulation in at least one respect but have the same constitutent members,
iv) differ in the liposome formulation in at least one respect but have the same constitutent members at the same relative amounts,
v) have the same liposome formulation in at least one respect but have the different constitutent members (with the relative amounts being constant or differing).

The efficacy of the foregoing methods has been particularly demonstrated herein for anti-cancer drugs, specifically for various combinations of doxil; doxorubicin; 5-fluorouracil; gemcitabine; irinotecan; vincristine; mifamurtide; cytarbine; and daunarubicin when breast cancer cells were used. Accordingly, in some embodiments relating to other cancer types or for personalized medicine approaches using a patient's own cells, the drug combinations or candidate combinations comprise at least two of doxil; doxorubicin; 5-fluorouracil; gemcitabine; irinotecan; vincristine; mifamurtide; cytarbine; and daunarubicin. In some embodiments of any of the aspects, the drug combinations or candidate combinations comprise a. doxorubicin and b. at least one of 5-fluorouracil, gemcitabine, and irinotecan. In some embodiments of any of the aspects, the drug combinations or candidate combinations consist of combinations whose elements/members are selected from doxil; doxorubicin; 5-fluorouracil; gemcitabine; irinotecan; vincristine; mifamurtide; cytarbine; and daunarubicin. In some embodiments of any of the aspects, the drug combinations or candidate combinations consist of combinations of a. doxorubicin and b. at least one of 5-fluorouracil, gemcitabine, and irinotecan.

As used herein, the terms “candidate compound” or “candidate agent” refer to a compound, substance, agent, and/or compositions or formulation thereof that are to be screened, e.g., for their Hill coefficient in combination with other compounds, substances, agents, and/or compositions or formulations thereof. Candidate compounds and/or agents can be produced recombinantly using methods well known to those of skill in the art (see Sambrook et al., Molecular Cloning: A Laboratory Manual (2 ed.), Cold Spring Harbor Laboratory Press, Cold Spring Harbor, N.Y., USA (1989)) or synthesized. Candidate compounds and agents can be screened for their Hill coefficient in combinations as described herein. In one embodiment of any of the aspects, candidate agents are screened using the assays described above herein.

As used herein, the terms “compound” or “agent” are used interchangeably and refer to molecules and/or compositions including, but not limited to chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; nucleic acids; nucleic acid analogs and derivatives; extracts made from biological materials such as bacteria, plants, fungi, or animal cells or tissues; naturally occurring or synthetic compositions; peptides; aptamers; and antibodies and intrabodies, or fragments thereof.

Compounds can be tested at any concentration that can modulate expression or protein activity relative to a control over an appropriate time period. In some embodiments of any of the aspects, compounds are tested at concentrations in the range of about 0.1 nM to about 1000 mM. In one embodiment, the compound is tested in the range of about 0.1 μM to about 20 μM, about 0.1 μM to about 10 μM, or about 0.1 μM to about 5 μM. In one embodiment, compounds are tested at 1 μM. Depending upon the particular embodiment being practiced, the test compounds can be provided free in solution, or may be attached to a carrier, or a solid support, e.g., beads. A number of suitable solid supports may be employed for immobilization of the test compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc.

In some embodiments of any of the aspects, the candidate agent or agent is an anti-cancer agent or therapeutic. As used herein “anti-cancer agent” or “therapeutic” refers to any chemical or biological agent with therapeutic usefulness in the treatment of diseases characterized by abnormal cell growth. Such diseases include tumors, neoplasms and cancer as well as diseases characterized by hyperplastic growth. Examples of anti-cancer agents can include, e.g., chemotherapeutics, radiation therapy reagents, immunotherapies, targeted therapies, or hormone therapies.

As used herein the term “chemotherapeutic agent” refers to any chemical or biological agent with therapeutic usefulness in the treatment of diseases characterized by abnormal cell growth by inhibiting a cellular activity upon which the cancer cell depends for continued survival and/or proliferation. In some aspect of all the embodiments, a chemotherapeutic agent is a cell cycle inhibitor or a cell division inhibitor. Categories of chemotherapeutic agents that are useful in the methods of the invention include alkylating/alkaloid agents, antimetabolites, hormones or hormone analogs, and miscellaneous antineoplastic drugs. Most of these agents are directly or indirectly toxic to cancer cells. In one embodiment, a chemotherapeutic agent is a radioactive molecule. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al; Chemotherapy, Ch. 17 in Abeloff, Clinical Oncology 2nd ed. 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). In some embodiments, the chemotherapeutic agent can be a cytotoxic chemotherapeutic. The term “cytotoxic agent” as used herein refers to a substance that inhibits or prevents the function of cells and/or causes destruction of cells. The term is intended to include radioactive isotopes (e.g. At211, I131, I125, Y90, Re186, Re188, Sm153, Bi212, P32 and radioactive isotopes of Lu), chemotherapeutic agents, and toxins, such as small molecule toxins or enzymatically active toxins of bacterial, fungal, plant or animal origin, including fragments and/or variants thereof.

As used herein, the term “immunotherapy” refers to refers to any chemical or biological agent with therapeutic usefulness in the treatment of diseases characterized by abnormal cell growth by promoting, preserving, or increasing the activity of immune cells. Immunotherapies include immune checkpoint inhibitors, T-cell transfer therapy (e.g., CAR-T therapies), antibody therapies, treatment vaccines, and immune system modulators.

Immune checkpoint inhibitors inhibit one or more immune checkpoint proteins. The immune system has multiple inhibitory pathways that are critical for maintaining self-tolerance and modulating immune responses. For example, in T-cells, the amplitude and quality of response is initiated through antigen recognition by the T-cell receptor and is regulated by immune checkpoint proteins that balance co-stimulatory and inhibitory signals. In some embodiments of any of the aspects, a subject or patient is treated with at least one inhibitor of an immune checkpoint protein. As used herein, “immune checkpoint protein” refers to a protein which, when active, exhibits an inhibitory effect on immune activity, e.g., T cell activity. Exemplary immune checkpoint proteins can include PD-1 (e.g., NCBI Gene ID: 5133); PD-L1 (e.g., NCBI Gene ID: 29126); PD-L2 (e.g., NCBI Gene ID: 80380); TIM-3 (e.g., NCBI Gene ID: 84868); CTLA4 (e.g., NCBI Gene ID: 1493); TIGIT (e.g., NCBI Gene ID: 201633); KIR (e.g., NCBI Gene ID: 3811); LAG3 (e.g., NCBI Gene ID: 3902); DD1-α (e.g., NCBI Gene ID: 64115); A2AR (e.g., NCBI Gene ID: 135); B7-H3 (e.g., NCBI Gene ID: 80381); B7-H4 (e.g., NCBI Gene ID: 79679); BTLA (e.g., NCBI Gene ID: 151888); IDO (e.g., NCBI Gene ID: 3620); TDO (e.g., NCBI Gene ID: 6999); HVEM (e.g., NCBI Gene ID: 8764); GALS (e.g., NCBI Gene ID: 3965); 2B4 (belongs to the CD2 family of molecules and is expressed on all NK, γδ, and memory CD8+(αβ) T cells) (e.g., NCBI Gene ID: 51744); CD160 (also referred to as BY55) (e.g., NCBI Gene ID: 11126); and various B-7 family ligands. B7 family ligands include, but are not limited to, B7-1, B7-2, B7-DC, B7-H1, B7-H2, B7-H3, B7-H4, B7-H5, B7-H6 and B7-H7.

Non-limiting examples of immune checkpoint inhibitors (with checkpoint targets and manufacturers noted in parantheses) can include: MGA271 (B7-H3: MacroGenics); ipilimumab (CTLA-4; Bristol Meyers Squibb); pembrolizumab (PD-1; Merck); nivolumab (PD-1; Bristol Meyers Squibb); atezolizumab (PD-L1; Genentech); galiximab (B7.1; Biogen); IMP321 (LAG3: Immuntep); BMS-986016 (LAG3; Bristol Meyers Squibb); SMB-663513 (CD137; Bristol-Meyers Squibb); PF-05082566 (CD137; Pfizer); IPH2101 (KIR; Innate Pharma); KW-0761 (CCR4; Kyowa Kirin); CDX-1127 (CD27; CellDex); MEDI-6769 (Ox40; MedImmune); CP-870,893 (CD40; Genentech); tremelimumab (CTLA-4; Medimmune); pidilizumab (PD-1; Medivation); MPDL3280A (PD-L1; Roche); MEDI4736 (PD-L1; AstraZeneca); MSB0010718C (PD-L1; EMD Serono); AUNP12 (PD-1; Aurigene); avelumab (PD-L1; Merck); durvalumab (PD-L1; Medimmune); IMP321, a soluble Ig fusion protein (Brignone et al., 2007, J. Immunol. 179:4202-4211); the anti-B7-H3 antibody MGA271 (Loo et al., 2012, Clin. Cancer Res. July 15 (18) 3834); TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94); anti-CTLA-4 antibodies described in U.S. Pat. Nos. 5,811,097; 5,811,097; 5,855,887; 6,051,227; 6,207,157; 6,682,736; 6,984,720; and 7,605,238; tremelimumab, (ticilimumab, CP-675,206); ipilimumab (also known as 10D1, MDX-D010); PD-1 and PD-L1 blockers described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699; nivolumab (MDX 1106, BMS 936558, ONO 4538); lambrolizumab (MK-3475 or SCH 900475); CT-011; AMP-224; and BMS-936559 (MDX-1105-01). The foregoing references are incorporated by reference herein in their entireties.

As used herein, the term “targeted therapy” refers to any chemical or biological agent with therapeutic usefulness in the treatment of diseases characterized by abnormal cell growth by inhibiting a cellular activity or element which increases the survival, growth, or proliferation of a cancer cell. These activities or elements are usually unique to cancer cells, e.g., as compared to the cells which the cancer arises from. Targeted therapies can include small molecule and antibody reagents.

As used herein, the term “hormone therapy” refers to any chemical or biological agent with therapeutic usefulness in the treatment of diseases characterized by abnormal cell growth by inhibiting the production or activity of a hormone that promotes cancer cell survival and/or proliferation.

Exemplary anti-cancer agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an antibody (e.g., alemtuzamab, bevacizumab (Avastin®), gemtuzumab, nivolumab (Opdivo®), pembrolizumab (Keytruda®), rituximab (Rituxan®), traztuzumab (Herceptin®) tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide (Revlimid®)), a kinase inhibitor (e.g., palbociclib (Ibrance®), or a hormone therapy (e.g., abiraterone acetate (Zytiga®)). General chemotherapeutic agents include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®, Etopophos®, Toposar®), fludarabine phosphate (Fludara®), 5-fluorouracil (Adrucil®, Efudex®), flutamide (Eulexin®), tezacitibine, gemcitabine (difluorodeoxycitidine), hydroxyurea (Hydrea®), ibrutinib (Imbruvica®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®). Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®, Tepadina®), busulfan (Busilvex®, Myleran®), improsulfan, piposulfan, carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®). Exemplary mTOR inhibitors include, e.g., temsirolimus; ridaforolimus (formally known as deferolimus, (1R,2R,45)-4-[(2R)-2 [(1R,95,125,15R,16E,18R,19R,21R,235,24E,26E,28Z,305,325,35R)-1,18-dihydroxy-19,30-dimethoxy-15,17,21,23,29,35-hexamethyl-2,3,10,14,20-pentaoxo-11,36-dioxa-4-azatricyclo[30.3.1.04′9] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RADOOl); rapamycin (AY22989, Sirolimus®); simapimod (CAS 164301-51-3); emsirolimus, (5-{2,4-Bis[(35,)-3-methylmorpholin-4-yl]pyrido[2,3-(i]pyrimidin-7-yl}-2-methoxyphenyl)methanol (AZD8055); 2-Amino-8-[iraw5,-4-(2-hydroxyethoxy)cyclohexyl]-6-(6-methoxy-3-pyridinyl)-4-methyl-pyrido[2,3-JJpyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-a-aspartylL-serine- (SEQ ID NO: 39), inner salt (SF1126, CAS 936487-67-1), and XL765. Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics). Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin. Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary proteosome inhibitors include bortezomib (Velcade®); carfilzomib (PX-171-007, (5)-4-Methyl-N-((5)-1-(((5)-4-methyl-1-((R)-2-methyloxiran-2-yl)-1-oxopentan-2-yl)amino)-1-oxo-3-phenylpropan-2-yl)-2-((5,)-2-(2-morpholinoacetamido)-4-phenylbutanamido)-pentanamide); marizomib (NPT0052); ixazomib citrate (MLN-9708); delanzomib (CEP-18770); and O-Methyl-N-[(2-methyl-5-thiazolyl)carbonyl]-L-seryl-O-methyl-N-[(11S′)-2-[(2R)-2-methyl-2-oxiranyl]-2-oxo-1-(phenylmethyl)ethyl]-L-serinamide (ONX-0912). Additional exemplary anti-cancer agents also include AMG479, vorinostat, ABT-737, PI-103; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaIl (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation.

In some embodiments of any of the aspects, a combination or candidate combination as described herein can comprise one or more adjuvants. As used herein in the context immune responses the term “adjuvant” refers to any substance that produces a more robust immune response. When incorporated into a therapeutic compositions, an adjuvant acts generally to accelerate, prolong, or enhance the quality of specific immune responses. Adjuvants are known in the art and can include, e.g., potassium alum; aluminium hydroxide; aluminium phosphate; calcium phosphate hydroxide; paraffin oil; Adjuvant 65; Plant saponins from Quillaja, soybean, or Polygala senega; IL-1; IL-2; IL-12; Freund's complete adjuvant; Freund's incomplete adjuvant; and squalene.

In some embodiments of any of the aspects, the adjuvant is a TLR4 adjuvant, e.g., a TLR4 agonist. As used herein, “TLR4”, “Toll-like receptor 4”, of “CD284” refers to a transmembrane protein of the toll-like receptor family that recognizes lipopolysaccharide (LPS), as well as viral proteins, polysacchairdes, and endogenous LDL, beta-defensins, and HSP. Sequences for TLR4 are known for a number of species, e.g., human TLR7 (NCBI Gene ID: 7099) mRNA sequences (NM_016562.3) and polypeptide sequences (NP_057646.1).

As used herein, the term “agonist” refers to an agent which increases the expression and/or activity of the target by at least 10% or more, e.g. by 10% or more, 50% or more, 100% or more, 200% or more, 500% or more, or 1000% or more. The efficacy of an agonist of, for example, TLR4, e.g. its ability to increase the level and/or activity of TLR4 can be determined, e.g. by measuring the level of an expression product of TLR4 and/or the activity of TLR4. Methods for measuring the level of a given mRNA and/or polypeptide are known to one of skill in the art, e.g. RT-PCR with primers can be used to determine the level of RNA, and Western blotting with an antibody can be used to determine the level of a polypeptide. Antibodies to TLR4 are commercially available, e.g., Cat. No. ab13556 and ab22048 from Abcam (Cambridge, Mass.). Assays for measuring the activity of TLR4, e.g. the increases in NF-κB and cytokine production in response to LPS detection are known in the art.

Agonists of TLR4 are known in the art and can include, by way of non-limiting example, monophosphoryl Lipid A (MPLA); RC-529; QS-21; SLA; SLA-SE; GLE-(SE), E6030, OM-174, DETOX, CCL-34; 8-(furan-2-yl) substituted pyrimido[5,4-b]indole analog (2B182C); and glucopyranosyl lipid A (GLA). Agonists of TLR4 are further described, e.g, at Toussi et al. Vaccines (Basel) 2014 2:323-53; Sato-Kaneko et al. Front. Immunol 2020 doi.org/10.3389/fimmu.2020.01207; Reed et al. Curr Opin Immunol 2016 41:85-90; Gregg et al. mBio 2017 8:e00492-17; Liang et al. npj Vaccines 2019 4:19; Chou et al. Scientific Reports 2020 10:8422 each of which is incorporated by reference herein in its entirety.

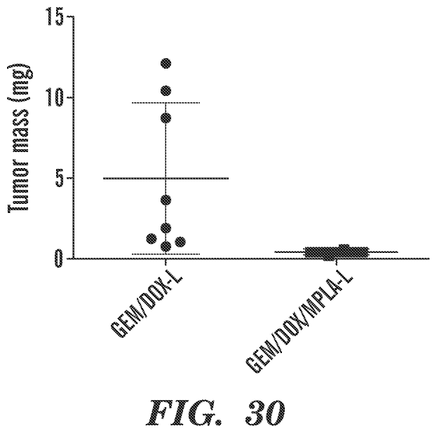

In some embodiments of any of the aspects, the TLR4 adjuvant is monophosphoryl lipid A (MPLA). As shown in Example 2 (see, e.g., FIG. 30), inclusion of a TLR4 adjuvant in combination with gemcitabine and doxorubicin showed a striking increase in the efficacy of tumor mass reduction.

In the Examples provided herein, a composition comprising liposomes, each liposome comprising both gemcitabine and doxorubicin was found to be particularly effective in treating cancer. Accordingly, in some embodiments of any of the aspects, described herein is a liposomal composition comprising individual liposomes each comprising both gemcitabine and doxorubicin. In some embodiments of any of the aspects, described herein is a liposomal composition comprising individual liposomes each comprising both gemcitabine and doxorubicin for use in a method of treating cancer. In some embodiments of any of the aspects, described herein is a method of treating cancer in a subject in need thereof, the method comprising administering to the subject a liposomal composition comprising individual liposomes each comprising both gemcitabine and doxorubicin.

In some embodiments of any of the aspects, the gemcitabine and doxorubicin are present at a molar ratio of from about 0.25:1 to about 4:1, respectively. In some embodiments of any of the aspects, the gemcitabine and doxorubicin are present at a molar ratio of from 0.25:1 to 4:1. In some embodiments of any of the aspects, the gemcitabine and doxorubicin are present at a molar ratio of from about 0.5:1 to about 2:1. In some embodiments of any of the aspects, the gemcitabine and doxorubicin are present at a molar ratio of from 0.5:1 to 2:1. In some embodiments of any of the aspects, the gemcitabine and doxorubicin are present at a molar ratio of about 1:1. In some embodiments of any of the aspects, the gemcitabine and doxorubicin are present at a molar ratio of 1:1.

As used herein, the term “liposome” refers to a vesicular structure having lipid-containing membranes enclosing an aqueous interior. In cell biology, a vesicular structure is a hollow, lamellar, spherical structure, and provides a small and enclosed compartment, separated from the cytosol by at least one lipid bilayer. Liposomes can have one or more lipid membranes. Oligolamellar large vesicles and multilamellar vesicles have multiple, usually concentric, membrane layers and are typically larger than 100 nm. Liposomes with several nonconcentric membranes, i.e., several smaller vesicles contained within a larger vesicle, are termed multivesicular vesicles.

Liposomes can further comprise one or more additional lipids and/or other components such as sterols, e.g., cholesterol. Additional lipids can be included in the liposome compositions for a variety of purposes, such as to prevent lipid oxidation, to stabilize the bilayer, to reduce aggregation during formation or to attach ligands onto the liposome surface. Any of a number of additional lipids and/or other components can be present, including amphipathic, neutral, cationic, anionic lipids, and programmable fusion lipids. Such lipids and/or components can be used alone or in combination. One or more components of the liposome can comprise a ligand, e.g., a targeting ligand. Liposome compositions can be prepared by a variety of methods that are known in the art and described in the Examples herein.

In some embodiments of any of the aspects, the liposomes comprise DSPC, DSPE-mPEG2000, and cholesterol. In some embodiments of any of the aspects, the liposomes consist of or consist essentially of DSPC, DSPE-mPEG2000, cholesterol, and the two or more drugs of the combination. In some embodiments of any of the aspects, the lipid content of the liposomes is 56.4% DSPC, 5.3% DSPE-mPEG2000, 38.3% cholesterol.

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastatses. Cancers which migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NEIL; intermediate grade diffuse NHL; high grade immunoblastic NHL; high grade lymphoblastic NEIL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

In some embodiments of any of the aspects, the cancer is breast cancer. In some embodiments of any of the aspects, the cancer cell is a breast cancer cell.

In some embodiments of any of the aspects, the methods described herein relate to treating a subject having or diagnosed as having, e.g., cancer. Subjects having such conditions can be identified by a physician using current methods of diagnosing them. For example, symptoms and/or complications of cancer which characterize these conditions and aid in diagnosis are well known in the art and include but are not limited to, growth of a tumor, impaired function of the organ or tissue harboring cancer cells, etc. Tests that may aid in a diagnosis of, e.g. cancer include, but are not limited to, tissue biopsies and histological examination. A family history of cancer or exposure to risk factors for cancer (e.g. smoking or radiation) can also aid in determining if a subject is likely to have cancer or in making a diagnosis of cancer.

The compositions and methods described herein can be administered to a subject having or diagnosed as having, e.g., cancer or a chronic infection. In some embodiments of any of the aspects, the methods described herein comprise administering an effective amount of compositions described herein to a subject in order to alleviate a symptom of, e.g., a cancer. As used herein, “alleviating a symptom” of a condition is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of an agent or combination thereof needed to alleviate at least one or more symptom of the disease or disorder, and relates to a sufficient amount of pharmacological composition to provide the desired effect. The term “therapeutically effective amount” therefore refers to an amount of an agent or combination thereof that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active agent which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

In some embodiments, the technology described herein relates to a pharmaceutical composition comprising an agent or combination thereof as described herein, and optionally a pharmaceutically acceptable carrier. In some embodiments, the active ingredients of the pharmaceutical composition comprise an agent or combination thereof as described herein. In some embodiments, the active ingredients of the pharmaceutical composition consist essentially of an agent or combination thereof as described herein. In some embodiments, the active ingredients of the pharmaceutical composition consist of an agent or combination thereof as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. an agent or combination thereof as described herein.

In some embodiments, the pharmaceutical composition comprising an agent or combination thereof as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of an agent or combination thereof as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of an agent as disclosed herein can also be incorporated into the parenteral dosage forms of the disclosure, including conventional and controlled-release parenteral dosage forms.

Pharmaceutical compositions comprising an agent or combination thereof can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the an agent or combination thereof can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

Im some embodiments of any of the aspects, the agent or combination thereof described herein is administered as a monotherapy, e.g., another treatment for the disease (e.g., cancer) is not administered to the subject.

In some embodiments of any of the aspects, the methods described herein can further comprise administering a further agent and/or treatment to the subject, e.g. as part of a combinatorial therapy. In addition, the methods of treatment can further include the use of radiation or radiation therapy. Further, the methods of treatment can further include the use of surgical treatments.

In certain embodiments, an effective dose of a composition comprising an agent or combination thereof as described herein can be administered to a patient once. In certain embodiments, an effective dose of a composition comprising an agent or combination thereof can be administered to a patient repeatedly. For systemic administration, subjects can be administered a therapeutic amount of a composition comprising agent or combination thereof, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient(s). The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising agent or combination thereof can be administered over a period of time, such as over a 5 minute, 10 minute, 15 minute, 20 minute, or 25 minute period.

The dosage ranges for the administration of agent or combination thereof, according to the methods described herein depend upon, for example, the form, its potency, and the extent to which symptoms, markers, or indicators of a condition described herein are desired to be reduced, for example the percentage reduction desired for tumor size or growth. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of an agent or combination thereof in, e.g. the treatment of a condition described herein, or to induce a response as described herein (e.g. a decrease in tumor size or growth rate) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of cancer. When using an experimental animal model, efficacy of treatment is evidenced when a statistically significant change in a marker is observed.

A dose-response curve is created by measuring the level of some output, marker, variable, or response to multiple doses of a treatment (e.g., candidate drug/agent/combination). In some embodiments of any of the aspects, the dose-response curve is created by measuring cell survival, death, and/or growth. The Hill coefficient is an exponential variable in a dose-response curve that indicates the degree of change in the output, marker, variable, or response when the dose is incrementally increased. A steep rise in a dose-response curve is indicative of a high Hill coefficient. The Hill equation is given as follows:

$$Y = {( 1 + {( \frac{IC 50}{X} )}^{m} )}^{- 1}$$

with X as the drug concentration and Y as the fractional inhibition, and m is known as the Hill coefficient and is determined according to the fit. The number of different concentrations that must be tested to calculate a Hill coefficient will vary depending on the steepness of the curve at the selected concentrations, as concentrations located at the steepest part of the curve will have the most impact on accuracy. In some embodiments of any of the aspects, the response is determined for enough different concentrations to yield an accuracy of m of at least 10%. In some embodiments of any of the aspects, the response is determined for at least 2 different concentrations. In some embodiments of any of the aspects, the response is determined for at least 3 different concentrations. In some embodiments of any of the aspects, the response is determined for at least 4 different concentrations. In some embodiments of any of the aspects, the response is determined for at least 5 different concentrations. In some embodiments of any of the aspects, the response is determined for at least 6 different concentrations. In some embodiments of any of the aspects, the response is determined for at least 7 different concentrations. In some embodiments of any of the aspects, the response is determined for at least 8 different concentrations. In some embodiments of any of the aspects, the response is determined for at least 9 different concentrations. In some embodiments of any of the aspects, the response is determined for at least 10 different concentrations.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of, e.g., cancer. A subject can be male or female.

A subject can be one who has been previously diagnosed with or identified as suffering from or having a condition in need of treatment (e.g. cancer) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

In some embodiments, the methods described herein relate to measuring, detecting, or determining the level of at least one response. As used herein, the term “detecting” or “measuring” can refer to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, the drug described herein is exogenous. In some embodiments of any of the aspects, the drug described herein is ectopic. In some embodiments of any of the aspects, the drug described herein is not endogenous.

The term “exogenous” refers to a substance present in a cell other than its native source. The term “exogenous” when used herein can refer to a substance a that has been introduced by a process involving the hand of man into a biological system such as a cell or organism in which it is not normally found. Alternatively, “exogenous” can refer to a substance that has been introduced by a process involving the hand of man into a biological system such as a cell or organism in which it is found in relatively low amounts and one wishes to increase the amount of the substance in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes a substance, such as a polypeptide or nucleic acid that is not naturally found or expressed in a given cell in its natural environment.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder associated with a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. In some embodiments of any of the aspects, prophylactic treatment is not prevention of all symptoms or signs of a disease.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

In some embodiments, the present technology may be defined in any of the following numbered paragraphs:
1. A method of treating cancer in a subject in need thereof with a drug combination, the method comprising administering to the subject a drug combination having an in vitro dose response Hill coefficient greater than 0.8.
2. A method of treating cancer in a subject in need thereof with a drug combination, the method comprising:
   a. contacting cancer cells in vitro with at least two different candidate combinations of the candidate drugs;
   b. measuring the in vitro dose response of the cancer cells to each candidate combination of step a;
   c. calculating the Hill coefficient from the dose response measured in step b;
   d. administering the combination with the largest Hill coefficient to the subject.
3. A method of treating cancer in a subject in need thereof with a drug combination, the method comprising administering to the subject a drug combination determined to have the largest in vitro dose response Hill coefficient from among a group of different drug combinations.
4. A method of selecting the most therapeutically effective combination of anti-cancer drugs from a pool of candidate drugs, the method comprising:
   a. contacting cancer cells in vitro with at least two different candidate combinations of the candidate drugs;
   b. measuring the in vitro dose response of the cancer cells to each candidate combination of step a;
   c. calculating the Hill coefficient from the dose response measured in step b;
   d. selecting the combination with the largest Hill coefficient as the most therapeutically effective of the candidate combinations.
5. A method of manufacturing a therapeutically effective combination of anti-cancer drugs from a pool of candidate drugs, the method comprising:
   a. forming at least two different candidate combinations of candidate drugs from a pool of candidate drugs;
   b. contacting cancer cells in vitro with the at least two different candidate combinations of the candidate drugs;
   c. measuring the in vitro dose response of the cancer cells to each candidate combination in step b;
   d. calculating the Hill coefficient from the dose response measured in step c;
   e. selecting the combination with the largest Hill coefficient as the most therapeutically effective of the candidate combinations; and
   f. providing the combination selected in step e as the therapeutically effective combination of anti-cancer drugs.
6. The method of any of the preceding paragraphs, wherein the largest Hill coefficient is greater than 0.8.
7. The method of any of the preceding paragraphs, wherein the largest Hill coefficient is greater than 1.0.
8. The method of any of the preceding paragraphs, wherein the largest Hill coefficient is greater than 1.5.
9. The method of any of paragraphs 2-7, wherein the cancer cells are primary cancer cells obtained from a/the subject.
10. The method of any of paragraphs 2-7, wherein the cancer cells are primary cancer cells obtained from a/the subject during treatment or diagnosis.
11. The method of any of paragraphs 2-7, wherein the cancer cells are primary cancer cells obtained from a/the subject no more than 3 months prior to the determination of the Hill coefficients.
12. The method of any of the preceding paragraphs, wherein the combination or candidate combination is a pairwise combination.
13. The method of any of the preceding paragraphs, wherein the combination or candidate combination is a combination of three, four, or more drugs or candidate drugs.
14. The method of any of the preceding paragraphs, wherein the drug combination or candidate combination is a. Doxorubicin and b. at least one of 5-fluorouracil, gemcitabine, and irinotecan.
15. The method of any of the preceding paragraphs, wherein the drug combination or candidate combination is at least two of:
   doxil; doxorubicin; 5-fluorouracil; gemcitabine; irinotecan; vincristine; mifamurtide; cytarbine; and daunarubicin.
16. The method of any of the preceding paragraphs, wherein the drug combination or candidate combination is provided in a liposome, wherein each member of the combination is present in the liposome.
17. The method of any of the preceding paragraphs, wherein the drug combination or candidate combination is provided in a mixture of liposomes, wherein each liposome comprises only one member of the combination.
18. The method of any of the preceding paragraphs, wherein combinations or candidate combinations differ from other combinations or candidate combinations in the identity of the drugs therein, the relative dose of the drugs therein, and/or the liposome formulation.
19. A method of treating cancer in a subject in need thereof, the method comprising administering to the subject a liposomal composition comprising individual liposomes each comprising both gemcitabine and doxorubicin.
20. The method of paragraph 19, wherein the gemcitabine and doxorubicin are present at a molar ratio of from 0.5:1 to 2:1.
21. The method of paragraph 19, wherein the gemcitabine and doxorubicin are present at a molar ratio of about 1:1.
22. The method of any of the preceding paragraphs, wherein the cancer is breast cancer.
23. A liposomal composition comprising individual liposomes each comprising both gemcitabine and doxorubicin.
24. The composition of paragraph 23, wherein the gemcitabine and doxorubicin are present at a molar ratio of from 0.5:1 to 2:1.
25. The composition of paragraph 23, wherein the gemcitabine and doxorubicin are present at a molar ratio of about 1:1.
26. The composition of any of paragraphs 23-25, for use in a method of treating cancer.
27. The composition of paragraph 26, wherein the cancer is breast cancer.

In some embodiments, the present technology may be defined in any of the following numbered paragraphs:
1. A method of treating cancer in a subject in need thereof with a drug combination, the method comprising administering to the subject a drug combination having an in vitro dose response Hill coefficient greater than 0.8.
2. A method of treating cancer in a subject in need thereof with a drug combination, the method comprising:
   a. contacting cancer cells in vitro with at least two different candidate combinations of the candidate drugs;
   b. measuring the in vitro dose response of the cancer cells to each candidate combination of step a;
   c. calculating the Hill coefficient from the dose response measured in step b;
   d. administering the combination with the largest Hill coefficient to the subject.
3. A method of treating cancer in a subject in need thereof with a drug combination, the method comprising administering to the subject a drug combination determined to have the largest in vitro dose response Hill coefficient from among a group of different drug combinations.
4. A method of selecting the most therapeutically effective combination of anti-cancer drugs from a pool of candidate drugs, the method comprising:
   a. contacting cancer cells in vitro with at least two different candidate combinations of the candidate drugs;
   b. measuring the in vitro dose response of the cancer cells to each candidate combination of step a;
   c. calculating the Hill coefficient from the dose response measured in step b;
   d. selecting the combination with the largest Hill coefficient as the most therapeutically effective of the candidate combinations.
5. A method of manufacturing a therapeutically effective combination of anti-cancer drugs from a pool of candidate drugs, the method comprising:
   a. forming at least two different candidate combinations of candidate drugs from a pool of candidate drugs;
   b. contacting cancer cells in vitro with the at least two different candidate combinations of the candidate drugs;
   c. measuring the in vitro dose response of the cancer cells to each candidate combination in step b;
   d. calculating the Hill coefficient from the dose response measured in step c;
   e. selecting the combination with the largest Hill coefficient as the most therapeutically effective of the candidate combinations; and
   f. providing the combination selected in step e as the therapeutically effective combination of anti-cancer drugs.
6. The method of any of the preceding paragraphs, wherein the largest Hill coefficient is greater than 0.8.
7. The method of any of the preceding paragraphs, wherein the largest Hill coefficient is greater than 1.0.
8. The method of any of the preceding paragraphs, wherein the largest Hill coefficient is greater than 1.5.
9. The method of any of paragraphs 2-7, wherein the cancer cells are primary cancer cells obtained from a/the subject.
10. The method of any of paragraphs 2-7, wherein the cancer cells are primary cancer cells obtained from a/the subject during treatment or diagnosis.
11. The method of any of paragraphs 2-7, wherein the cancer cells are primary cancer cells obtained from a/the subject no more than 3 months prior to the determination of the Hill coefficients.
12. The method of any of the preceding paragraphs, wherein the combination or candidate combination is a pairwise combination.
13. The method of any of the preceding paragraphs, wherein the combination or candidate combination is a combination of three, four, or more drugs or candidate drugs.
14. The method of any of the preceding paragraphs, wherein the combination or candidate combination comprises one or more adjuvants.
15. The method of paragraph 14, wherein the one or more adjuvants comprise one or more TLR4 adjuvants.
16. The method of paragraph 15, wherein the TLR4 adjuvant is monophosphoryl lipid A (MPLA).
17. The method of any of the preceding paragraphs, wherein the drug combination or candidate combination is or comprises a. Doxorubicin and b. at least one of 5-fluorouracil, gemcitabine, and irinotecan.
18. The method of any of the preceding paragraphs, wherein the drug combination or candidate combination is or comprises at least two of:
   doxil; doxorubicin; 5-fluorouracil; gemcitabine; irinotecan; vincristine; mifamurtide; cytarbine; and daunarubicin.
19. The method of any of the preceding paragraphs, wherein the drug combination or candidate combination is provided in a liposome, wherein each member of the combination is present in the liposome.
20. The method of any of the preceding paragraphs, wherein the drug combination or candidate combination is provided in a mixture of liposomes, wherein each liposome comprises only one member of the combination.
21. The method of any of the preceding paragraphs, wherein combinations or candidate combinations differ from other combinations or candidate combinations in the identity of the drugs therein, the relative dose of the drugs therein, and/or the liposome formulation.
22. A method of treating cancer in a subject in need thereof, the method comprising administering to the subject a liposomal composition comprising individual liposomes each comprising both gemcitabine and doxorubicin.
23. The method of paragraph 22, wherein the gemcitabine and doxorubicin are present at a molar ratio of from 0.5:1 to 2:1.
24. The method of paragraph 22, wherein the gemcitabine and doxorubicin are present at a molar ratio of about 1:1.
25. The method of any of paragraphs 22-24, wherein the composition further comprises one or more adjuvants.
26. The method of paragraph 25, wherein the one or more adjuvants comprise one or more TLR4 adjuvants.
27. The method of paragraph 26, wherein the TLR4 adjuvant is monophosphoryl lipid A (MPLA).
28. The method of any of the preceding paragraphs, wherein the cancer is breast cancer.
29. A liposomal composition comprising individual liposomes each comprising both gemcitabine and doxorubicin.
30. The composition of paragraph 29, wherein the gemcitabine and doxorubicin are present at a molar ratio of from 0.5:1 to 2:1.
31. The composition of paragraph 29, wherein the gemcitabine and doxorubicin are present at a molar ratio of about 1:1.
32. The composition of any of paragraphs 29-31, wherein the composition further comprises one or more adjuvants.
33. The composition of paragraph 32, wherein the one or more adjuvants comprise one or more TLR4 adjuvants.
34. The composition of paragraph 33, wherein the TLR4 adjuvant is monophosphoryl lipid A (MPLA). The composition of any of paragraphs 29-34, for use in a method of treating cancer.
35. The composition of paragraph 35, wherein the cancer is breast cancer.

### EXAMPLES

### Example 1: Design Principles of Drug Combinations for Chemotherapy

Combination chemotherapy is the leading clinical option for cancer treatment. The current approach to designing drug combinations includes in vitro optimization to maximize drug cytotoxicity and/or synergistic drug interactions. However, in vivo translatability of drug combinations is complicated by the disparities in drug pharmacokinetics and biodistribution. In vitro cellular assays also fail to represent the immune response that can be amplified by chemotherapy when dosed appropriately. Using three common chemotherapeutic drugs, 5-fluorouracil (5FU), gemcitabine (GEM), and irinotecan (IRIN), paired with another common drug and immunogenic cell death inducing agent, doxorubicin (DOX), we describe herein the in vitro parameters that predict in vivo outcomes of drug combinations using the highly aggressive orthotopic 4T1 model. Using liposomes to encapsulate drug combinations permitted uniform drug pharmacokinetics across the drug combinations, thus leading to the study of the inherent benefits of the drug pairs and comparisons to DOX liposomes representative of DOXIL®. Surprisingly, the Hill Coefficient (HC) of the in vitro dose-response curve provided a better prediction of in vivo efficacy than drug IC₅₀ or combination index. GEM/DOX liposomes exhibited a high HC in vitro and an increase in M1/M2 macrophage ratio in vivo. Hence, GEM/DOX liposomes were further investigated in a long-term survival study and compared against doxorubicin liposomes and gemcitabine liposomes. The results showed a doubling of median survival time of GEM/DOX liposomes when compared to DOX liposomes alone and represented a 3.4-fold increase when compared to untreated controls. These studies document the development of a more efficacious formulation than clinically representative liposomal doxorubicin for breast cancer treatment and a novel strategy for designing cancer drug combinations.

### Introduction

Combination chemotherapies have become the standard of care for treating various cancers¹⁻³. Chemotherapeutic regimens typically utilize drugs with non-overlapping mechanisms of action to minimize tumor drug resistance while maximizing tumor response; however, some also exhibit significantly worsened patient toxicity⁴⁻⁶ without much added therapeutic benefit. This is partially due to differences in clearance and distribution of each drug, which changes the ratiometric composition of the drugs that reach the tumor site and subsequently makes it challenging to predict the combination's activity. Drug delivery strategies have addressed this problem by utilizing nanoparticle encapsulation to control the release profile and pharmacokinetics of the drug combination, as well as increase tumor accumulation⁷. However, despite a handful of successful examples, nanoparticle drug combinations still suffer from unpredictable translation to the clinic^{8,9}, pointing to the need to re-evaluate how drug combinations are developed in vitro before translation begins. Currently, drug combinations are extensively optimized in vitro before testing in vivo, with heavy reliance on parameters such as drug IC₅₀ and combination index¹⁰ serving as benchmarks of success¹¹⁻¹⁴. Identifying other in vitro parameters to permit more comprehensive prediction of in vivo outcome remains a neglected area of research in spite of its potential impact on clinical translation of early stage therapeutics.

Using a panel of four common chemotherapeutic drugs, we sought to determine in vitro parameters that correlate with the in vivo performance of drug combinations. Doxorubicin (DOX) was selected due to its wide applicability in a range of cancers, as well as its ease of use in a liposomal form. It has the added benefit of causing immunogenic cell death in tumors, which is useful for immune activation against cancer at moderate doses¹⁵. DOX was evaluated in combination with a 5-fluorouracil prodrug (5FURW), gemcitabine (GEM), and irinotecan (IRIN). All drug combinations were encapsulated in liposomes to stabilize the drug pairs during in vivo translation. Liposomes offer an excellent means for controlling the relative pharmacokinetics of multiple drugs while also improving their circulation half-life¹⁶⁻¹⁸. In addition to providing a platform to help answer the key question posed in this study, liposomes also facilitate clinical translation of chemotherapeutic drugs. Liposomal drug delivery systems have already experienced considerable clinical and commercial success for single drugs¹⁹, in particular, the first commercial liposomal formulation Doxil® has been used for treating several cancers and continues to be used in clinical trials for combination with immunotherapy or other agents²⁰⁻²³. Several additional liposomal drugs including irinotecan (ONIVYDE®), vincristine (MARQIBO®), mifamurtide (MEPACT®), and daunorubicin (DaunoXome®) have also been approved by regulatory agencies and are currently used clinically. Many other drugs, including 5-fluorouracil²⁴, gemcitabine²⁵, and paclitaxel²⁶, have been encapsulated in liposomes and tested at the preclinical level. Finally, recent approval of Vyxeos®, a co-encapsulated liposomal formulation of cytarabine and daunorubicin for the treatment of acute myeloid leukemia, marks the start of a new generation of liposomes for the delivery of drug combinations as well²⁷.

Liposomal formulations were evaluated in vitro and in vivo in terms of toxicity, release, and pharmacokinetics. In vivo efficacy was studied in the orthotopic 4T1 murine breast cancer model, which advances aggressively and metastasizes to the lungs. Tumors were extracted for immune profiling to assess changes in tumor immune infiltrate, and the correlation between tumor mass and several different in vitro parameters was investigated. It is described herein that among all combination parameters tested, the Hill Coefficient (HC) of in vitro dose-response model served as the best predictor of in vivo efficacy. The lead liposomal formulation, as indicated by its HC, doubled the median survival time when compared to DOX liposomes, a clinically relevant formulation. These findings indicate that accounting for more parameters such as the Hill coefficient would help make better informed decisions in translational studies.

Results

Liposome Synthesis and Physical Characterization

Liposomes (56.4% 1,2-distearoyl-sn-glycero-3-phosphocholine (DSPC), 5.3% 1,2-distearoyl-sn-glycero-3-phosphoethanolamine-N-[methoxy(polyethylene glycol (PEG))-2000] (DSPE-PEG(2000)), 38.3% cholesterol) were synthesized using the thin film hydration technique. The lipid composition is similar to many used in clinical liposomal formulations. Drugs were encapsulated in liposomes (denoted with -L) and characterized for size and surface charge in milliQ water (Table 1). The incorporation of 5 mol % DSPE-PEG(2000) affords a negative surface potential of <−20 mV to all liposomes, which is necessary for improving circulation times and improving stability via electrostatic repulsion^{29,30}. Passive and active methods were used for loading of drugs (see description in the Methods), yet all liposomal formulations possessed comparable sizes and zeta potentials, demonstrating that drug loading did not alter the physical characteristics of the liposomes. The drug ratios in all combination liposomes were approximately equimolar unless mentioned otherwise.

TABLE 1
Summary of liposome physical and chemical properties.

| | | | | Zeta |
|---|---|---|---|---|
| | Loading | | | potential |
| Liposome | technique | Molar ratio | Size (nm) | (mV) |
| DOX-L | active | n/a | 77.3 ± 1.1 | −24.2 ± 1.7 |
| 5FURW/ | active | 0.95 | 75.1 ± 0.33 | −26.7 ± 1.6 |
| DOX-L | | | | |
| 5FURW/ | active | 2.6 | 72.0 ± 3.0 | −24.7 ± 1.9 |
| DOX-L_{R = 2.5} | | | | |
| GEM/DOX-L | passive + active | 0.76 | 70.3 ± 0.7 | −23.2 ± 2.8 |
| IRIN/DOX-L | active | 1.0 | 74.7 ± 0.9 | −27.7 ± 2.0 |

Liposomes Demonstrate Stable Encapsulation of Drugs

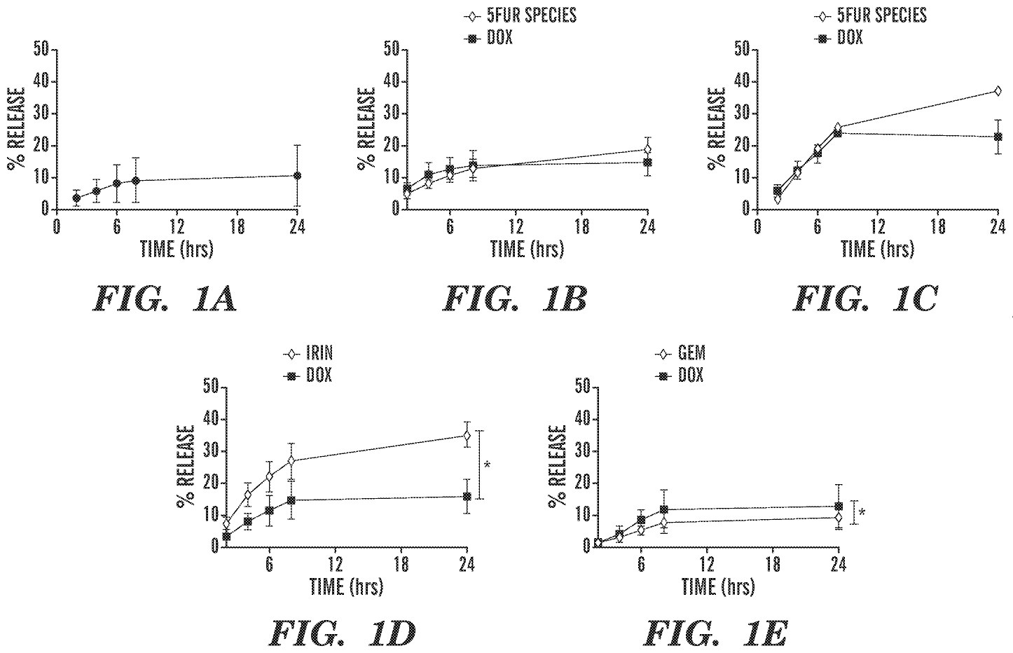

Drug release from all formulations was quantified over a period of 24 hours to measure liposome stability under conditions that simulate circulation (FIGS. 1A-1E). A control formulation of DOX-L showed slow DOX release, with approximately 10% released over 24 hours. In co-encapsulated liposomes, less than 25% of the encapsulated DOX dose was released over 24 hours. Other drugs exhibited release rates similar to DOX with the exception of IRIN, which was released faster than DOX (p<0.05). In the case of increased ratio (R=3.5) 5FURW/DOX-L, high loading of 5FURW resulted in accelerated release of 5FURW such that 40% was released in 24 hours. However, at equimolar loading, GEM/DOX-L and 5FURW/DOX-L both released less than 20% of each encapsulated drug species.

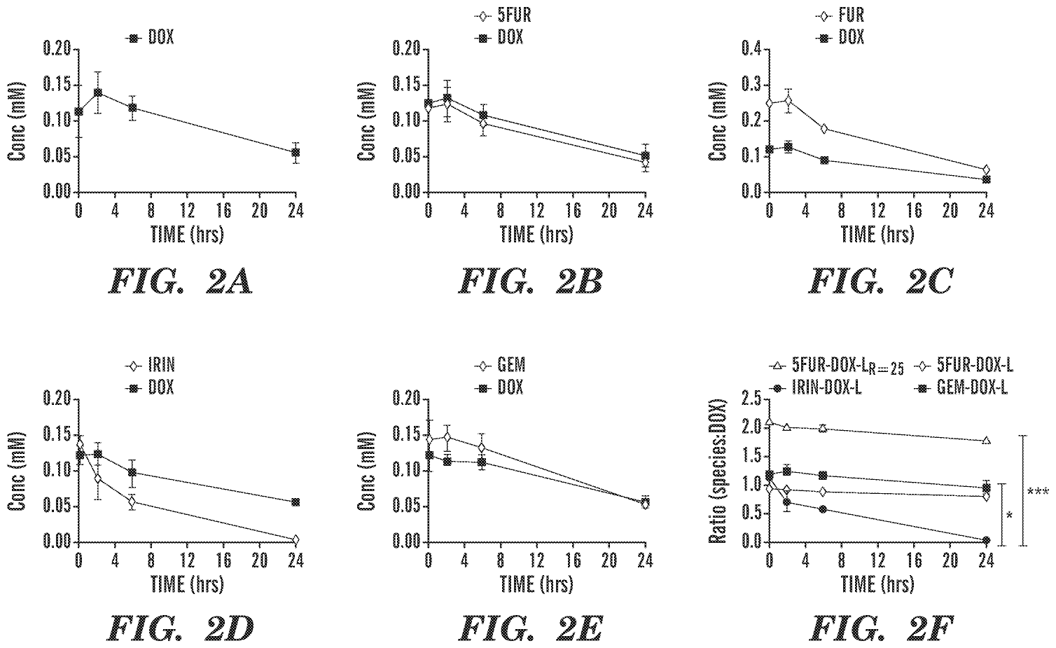

Upon intravenous injections in mice, all formulations exhibited prolonged circulation in blood for up to 24 hours in circulation (FIGS. 2A-2F). The pharmacokinetic parameters of each formulation were calculated using the PK Solver plugin in Microsoft Excel³¹ (Table 5). Calculations based on the DOX concentration showed that the half-life (t_{1/2}) of the formulations were similar, with values in a range from 12 to 21 hours, with an average of 17.5±3.4 hours. The drug ratios in blood remained mostly conserved over 24 hours. In the case of GEM/DOX-L and both formulations of 5FURW/DOX-L, the ratio of drugs stayed within 80% of their original value. The ratio of IRIN/DOX dropped to 6% of its original value over 24 hours likely due to faster release of IRIN from liposomes (FIGS. 1A-1E). However, the ratio remained above 50% of the original value for 8 hours after administration.

Cellular Inhibition Assays Demonstrate Efficacy and Drug Sensitivity Differences

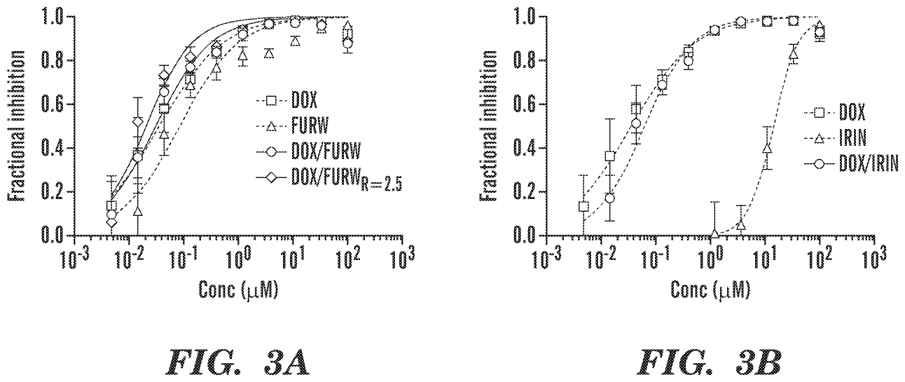

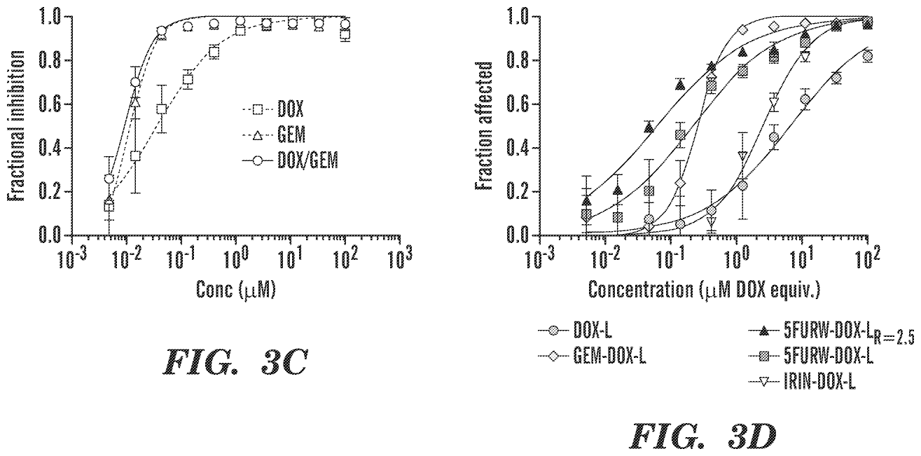

The drug pairs were evaluated for cytotoxicity using a two-dimensional culture of the murine breast cancer cell line 4T1. The dose-response data were fitted to the Hill equation for all free drugs, drug combinations, and liposomal formulations (FIGS. 3A-3D). Synergy was evaluated using the Combination Index (CI) from the Chou-Talalay method¹⁰. All drug combinations had a CI that fell between 1 and 2 (Table 3). In some cases, large differences in IC₅₀ values between drugs were present; such was the case of doxorubicin and irinotecan, which had IC₅₀ concentrations of 0.036 μM and 14.04 μM respectively (FIG. 3B). Combining DOX and IRIN with a 1:1 ratio resulted in an IC₅₀ close to the IC₅₀ of DOX, thereby improving heavily on the IC₅₀ of IRIN. In another case, neither 5FURW/DOX combination were different from DOX (p>0.05) (FIG. 3A). The only free single drug with lower IC₅₀ than DOX was GEM. A 1:1 combination of GEM and DOX did not boost the efficacy beyond single drug GEM, and thus the IC₅₀ of the combination was only marginally below GEM (FIG. 3C).

Interestingly, while GEM and IRIN have widely different IC₅₀ values, they have the steepest rise in their dose-response curves. This can be quantified by the Hill coefficient, an exponential variable in the dose-response curve that indicates the degree of change in cellular inhibition when the dose is incrementally increased. IC₅₀ and HC are fundamentally independent of the other. For example, GEM has both a low IC₅₀ and a high HC (12±0.3 nM and 1.8±0.09), whereas IRIN initially has a high IC₅₀ but a high HC as well (14±0.7 μM and 1.9±0.15). Table 3 gives a summary of the IC₅₀, CI, and HC of each drug both when given alone and in combination with DOX.

TABLE 3
Combination indices, IC₅₀ values, and Hill coefficients of
free drugs and free drug combinations

| Drug/ | | | Hill coefficient, |
|---|---|---|---|
| Drug combination | CI | IC₅₀ (μM) | HC |
| DOX | — | 0.036 ± 0.003 | 0.75 ± 0.04 |
| 5FURW | — | 0.084 ± 0.009 | 0.82 ± 0.06 |
| IRIN | — | 14.0 ± 0.65 | 1.90 ± 0.15 |
| GEM | — | 0.012 ± 0.0003 | 1.83 ± 0.09 |
| 5FURW/DOX_{R = 1} | 1.22 ± 0.11 | 0.031 ± 0.002 | 0.86 ± 0.05 |
| 5FURW/DOX_{R = 2.5} | 1.18 ± 0.12 | 0.021 ± 0.001 | 1.0 ± 0.08 |
| IRIN/DOX_{R = 1} | 1.70 ± 0.16 | 0.061 ± 0.004 | 0.99 ± 0.05 |
| GEM/DOX_{R = 1} | 1.03 ± 0.04 | 0.01 ± 0.0003 | 1.68 ± 0.07 |

The trend in HC from free drug experiments (FIG. 3A-3C) carries through to liposomal formulations as well (FIG. 3D). Liposomes containing GEM/DOX, 5FURW/DOX, or IRIN/DOX were tested against 4T1 cell cultures. The HC and IC₅₀ value trends were found to mirror the results of free drugs in vitro (Table 4). However, in comparison to free drugs, all liposomal formulations demonstrate an increase in IC₅₀. The release kinetics of the liposomes are likely to play a role in their toxicity. As shown in FIGS. 1A-1E, DOX is very stably encapsulated in liposomes. The IC₅₀ of liposomal DOX demonstrated a 166-fold increase when compared to that of its free form. Combinations with DOX exhibited an increase in IC₅₀ as well, although none were as pronounced as the difference between DOX-L and free DOX due to the tendency of co-loaded drugs to release faster.

TABLE 4
IC₅₀ values and Hill coefficients of liposomal formulations.

| | IC₅₀ (μM) | HC |
|---|---|---|
| DOX-L | 6.65 ± 0.93 | 0.67 ± 0.06 |
| 5FURW/DOX-L | 0.221 ± 0.02 | 0.67 ± 0.04 |
| 5FURW/DOX-L_{R = 2.5} | 0.063 ± 0.01 | 0.62 ± 0.03 |
| IRIN/DOX-L | 2.54 ± 0.24 | 1.2 ± 0.1 |
| GEM/DOX-L | 0.25 ± 0.01 | 1.9 ± 0.2 |

Tumor Growth Inhibition by Single-Dose Liposomal Formulations

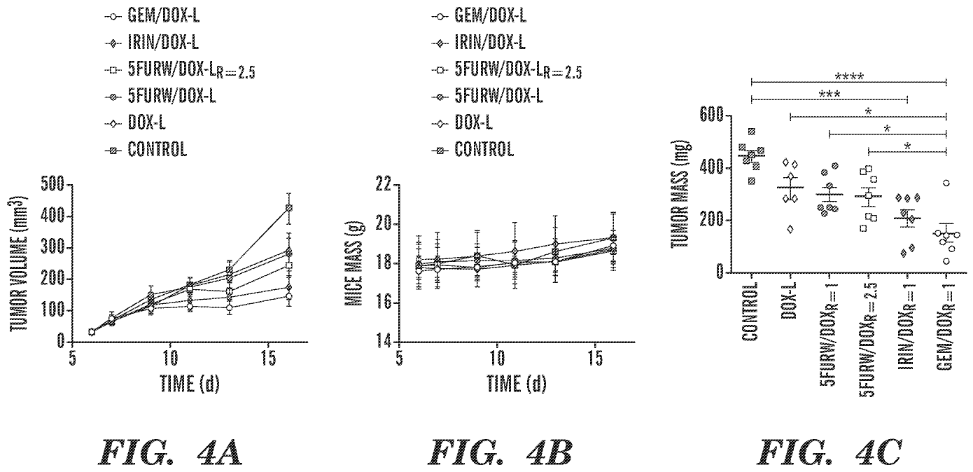

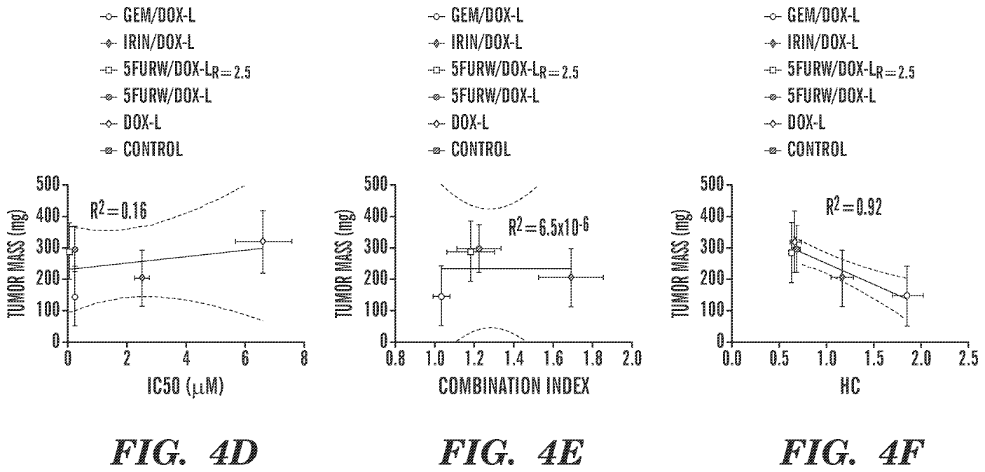

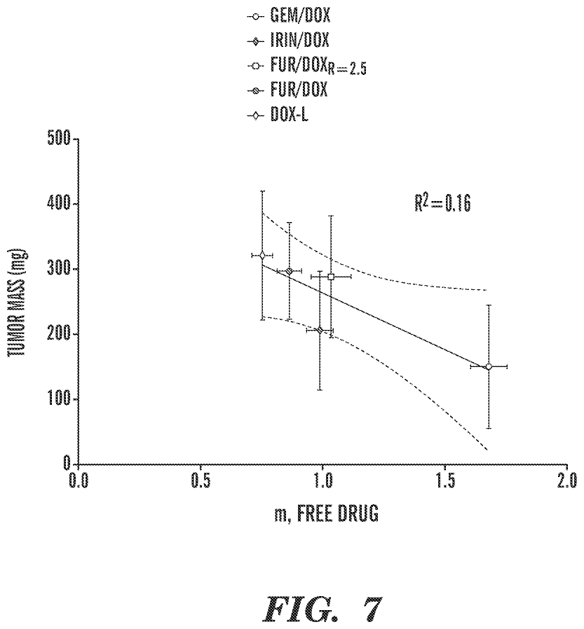

The efficacy of liposomal drug combinations was tested in vivo using an orthotopic 4T1 tumor model, an aggressive model that tends to metastasize to the lungs and hence makes a representative model for human breast cancer³². Mice were a given a single dose of liposomes with DOX content eight-fold lower than the maximum tolerated dose of Doxil³³ and the growth was followed for 10 days (FIG. 4A). The DOX dose was 3 mg/kg in all cases and the dose of the paired drug was fixed by the combination molar ratio. No weight loss was observed for any drug combination (FIG. 4B). DOX alone was only moderately effective in tumor reduction whereas GEM/DOX was most effective. GEM/DOX demonstrated a 71% tumor growth inhibition compared to controls and 56% reduction compared to DOX. IRIN/DOX also demonstrated 64% tumor growth inhibition compared to the control group and 45% compared to DOX. Tumor mass was recorded and agreed with tumor volume measurements (FIG. 4C) No correlation (R²=0.16) was found between tumor mass and in vitro IC₅₀ (FIG. 4D) or combination index (FIG. 4E). However, a strong correlation (R²=0.92) was found between HC of in vitro liposomal formulations and tumor mass (FIG. 4F). This correlation was also observed with free drug combinations (FIG. 7) indicating that drug sensitivity is a more representative method to predict in vivo response than IC₅₀ or combination index at the doses administered in the 4T1 tumor model.

Tumor Immune Infiltrate Profiling after Liposomal Treatment

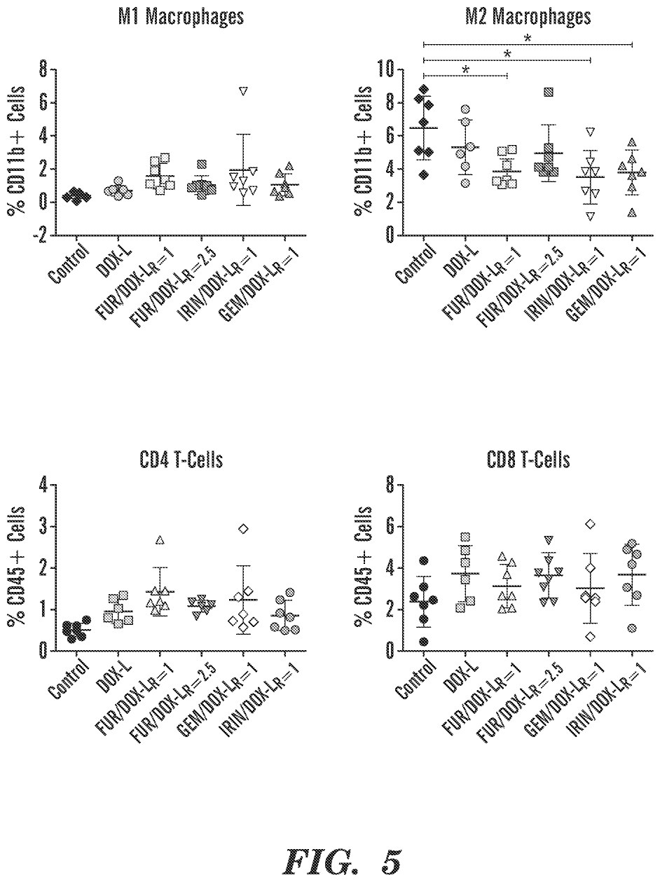

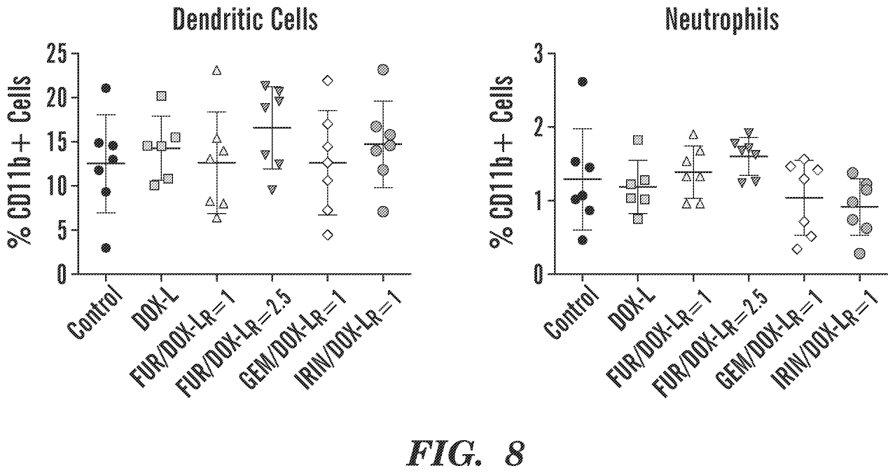

The impact of the liposomal formulations on the immune tumor environment were evaluated. The immunogenic effects of doxorubicin have been well-reported in the 4T1 model^{34,35}. DOX and other anthracyclines have been shown to elicit immunogenic cell death³⁶, but their effects when in combination with other chemotherapeutic drugs has not been well studied. Characteristic markers of classically activated (M1) and alternatively activated (M2) macrophages, cytotoxic (CD8+) T cells and helper T cells (CD4+CD25-), as well as dendritic cells and myeloid-derived suppressor cells were examined for upregulation upon administration of single-dose liposomal formulations (FIG. 5). No significant difference in the adaptive immune response between the control and treated groups was found, which may have been due to insufficient dosing^{34,37,38}. Dendritic cells and neutrophils in the treatment groups seemed to be relatively unchanged from the untreated control group (FIG. 8).

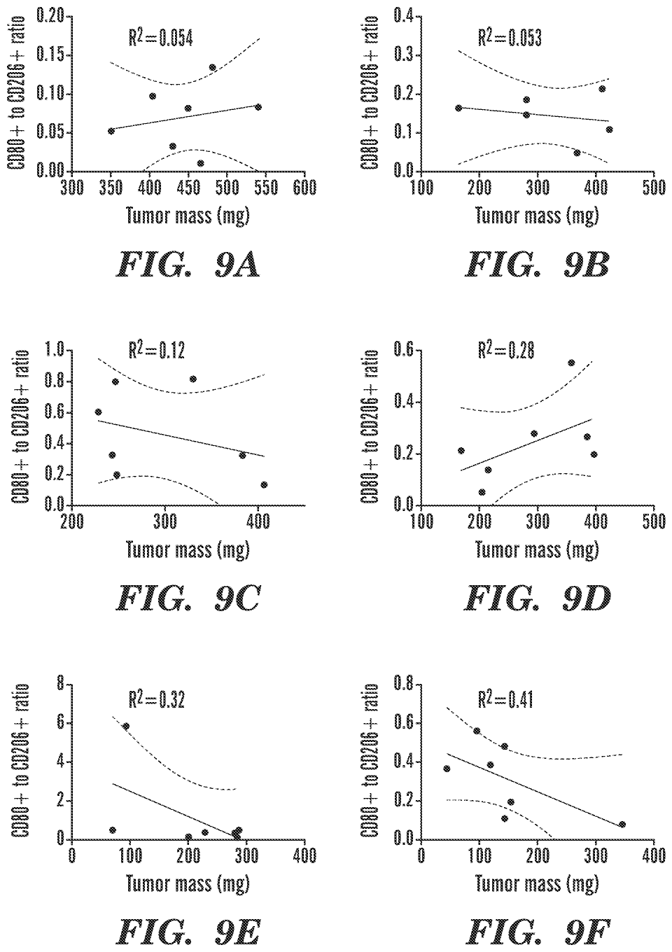

GEM/DOX and IRIN/DOX treated tumors, which had the greatest reduction in tumor volume, also had significantly decreased levels of M2 macrophages. Immunosuppressive tumor-associated M2 macrophages typically correlate with poor tumor prognosis, while their immunostimulatory counterpart M1 macrophages are associated with better tumor immune recognition³⁹. GEM/DOX and IRIN/DOX treated tumors had M2 levels that were 57.8% and 54.2% of the untreated average. DOX treatment alone did not produce significantly different levels of M1 or M2 macrophages. This suggests the drug pairings had an effect in elevating the immune response, as single DOX treatment did not seem to influence any tumor infiltrating cell phenotype. Within the GEM/DOX and IRIN/DOX groups, the M1/M2 macrophage ratios exhibited an inverse correlation with tumor mass (FIGS. 9A-9F).

Survival Study

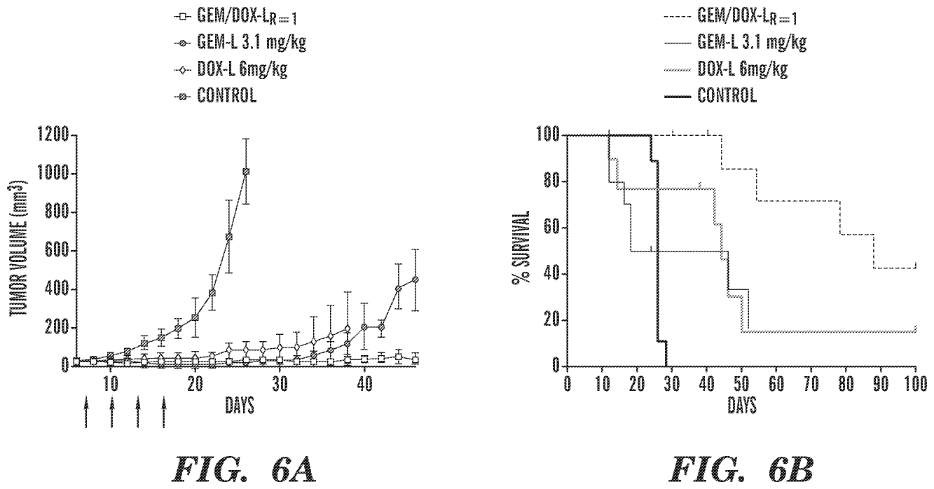

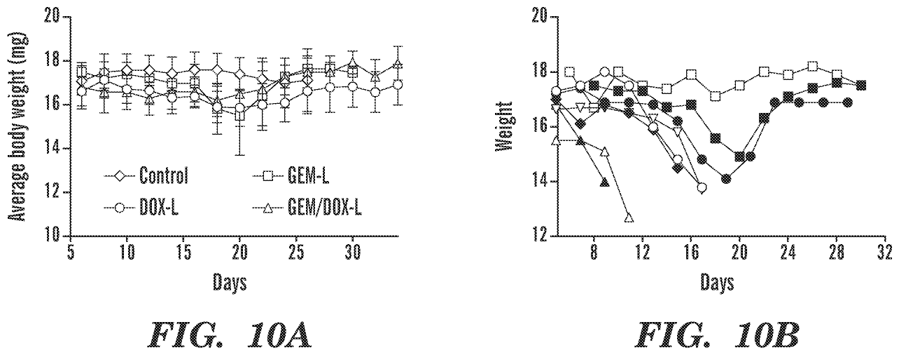

Given its efficacy after a single injection in vivo, a long-term survival study was conducted with GEM/DOX co-encapsulated liposomes. The GEM/DOX-L group was treated with 3 mg/kg DOX and 1.55 mg/kg GEM. Corresponding controls were DOX liposomes (DOX-L) alone at 6 mg/kg and GEM liposomes (GEM-L) alone at 3.1 mg/kg. High doses of DOX-L and GEM-L were chosen as controls to assess whether the combination is truly beneficial over individual drugs. DOX-L serves to represent the clinical formulation of pegylated liposomal doxorubicin, better known as Doxil. The cumulative dose delivered by GEM/DOX-L was 12 mg/kg DOX and 6.2 mg/kg GEM, which is well below reported dosages of Doxil (25 mg/kg)³³ and liposomal GEM (8 mg/kg)⁴⁰. GEM/DOX-L demonstrated remarkable tumor volume control (FIG. 6A), and GEM/DOX-L treated mice significantly outlived their DOX treated and GEM treated counterparts (FIG. 6B). The median survival doubled when comparing DOX-L to GEM/DOX-L from 44 days to 88 days (Table 7). This is exceptional tumor control especially in the highly aggressive 4T1 tumor model. Additionally, this represents a 238% increase in the lifespan compared to the untreated control group, and a 100% increase in lifespan compared to DOX-L. Five out of the original seven mice treated with GEM/DOX-L became long-term survivors (60 days) with cured primary tumors. Of these five mice, two eventually succumbed not to primary tumor growth but to lung metastases after 60 days from tumor inoculation, as evidenced by visible nodule formations on the lungs of mice analyzed post-mortem. However, three mice in the GEM/DOX-L group continued to survive until the study concluded at 100 days. In comparison, half of the GEM-L group did not survive during dosing (FIG. 10B) and there were no long-term survivors past 60 days. The mice in the DOX-L group were euthanized due to weight loss or tumor volume endpoints before 60 days, with one long term survivor past the 60-day benchmark.

Discussion

Using dual-drug loaded liposomes, we evaluated in vitro to in vivo translation of drug combinations in an immunocompetent model. By using liposomes, one of the most translatable drug carriers, we controlled drug ratio and distribution to understand what in vitro parameters could be used to predict in vivo performance. We used the highly metastatic 4T1 model in immune competent BALB/c mice to study how the immune system impacted tumor regression for a given drug pair. We chose to use a variety of drugs for co-encapsulation with doxorubicin, which is clinically approved for breast cancer and is a known immunostimulatory⁴¹.

The drug combinations were evaluated in vitro in terms of IC₅₀, Hill coefficient, and synergy before immune effects were studied in vivo. We paired doxorubicin with passively loaded gemcitabine, and actively loaded prodrug forms of 5-flourouracil¹². While GEM and 5FURW are both immunogenic drugs³⁵, we also co-loaded irinotecan, which does not have well-characterized immune effects in vivo. However, IRIN was notable because it had a much higher IC₅₀ than the other drugs, but also had a Hill coefficient greater than 1.

Liposomes were used as a model nanocarrier for controlling the pharmacokinetic parameters and drug release profile of the drug pairs. This would exclude these factors from consideration when studying the in vitro to in vivo translation of the drugs. We observed negative zeta potentials on all liposomal formulations, which helps to prevent opsonization in vivo and leads to reduced aggregation of particles in solution through electrostatic repulsion⁴². The release profile of all formulations showed DOX to be stably encapsulated, as well as GEM and 5FURW. IRIN released substantially quicker than DOX, but even so less than 40% had released within 24 hours. Combined with sustained release, we were able to observe typical liposomal half-lives for all formulations as well as stable drug ratios. With consistent pharmacokinetic parameters and release profiles, we were able to specifically study the impact of in vitro drug activity and immune response on the final tumor efficacy.

Surprisingly, tumor response did not correlate with decreasing IC₅₀ or CI, but rather with Hill coefficient (HC). A steep dose-response curve indicates that the tumor cells are more sensitive to the drug or drug combination. With a greater cellular response for every incremental change in drug concentration, combinations with higher HC were more likely to induce a stronger anti-tumor effect. We were able to confirm the translational significance of high HC using our selected drugs in an in vitro 4T1 dose-response experiments and the orthotopic 4T1 tumor model. The 4T1 model distinguishes itself as being extremely aggressive with a quick doubling time both in vitro and in vivo, which may have had an impact on our results. In addition, we verified the importance of high HC.

A drug combination with high HC was sufficient to control the growth of the highly aggressive 4T1 model and that can apply to other cell lines and tumor models as well. The Hill coefficient is traditionally known as an indicator of “interactivity” among binding ligands to multiple sites on a receptor⁴³. A Hill coefficient of 1 represents independent binding of a ligand to one specific site on the receptor, whereas values greater than one indicate cooperative binding, in which the binding of one ligand encourages the binding of other ligands to the receptor⁴⁴. In the case of combination chemotherapy, this likely corresponds to rapid cancer cell death with larger increases in cellular inhibition for a small change in drug concentration. IC₅₀ itself fundamentally changes with cell division rates, cell seeding density, and drug incubation time, making it an extrinsic variable⁴⁵. Thus, more parameters such as the Hill coefficient would help make more informed decisions when translating from in vitro to an animal model.

When the formulations were studied in vivo, GEM/DOX and IRIN/DOX had the sharpest reduction in tumor volume after one administration. In addition to having a high Hill coefficient, GEM/DOX showed significantly lower levels of M2 macrophages compared to the untreated control and produced a significantly longer overall survival in an immunocompetent 4T1 murine breast cancer model. M2 macrophages in the tumor microenvironment become tumor-associated macrophages (TAMs) that facilitate tumor growth by stimulating tumor angiogenesis and metastasis⁴⁶. Most reports identify TAMs as the M2 phenotype and correlate it with poor prognosis. This has been shown in ovarian cancer⁴⁷, breast cancer⁴⁸, liver cancer⁴⁹, and non-small cell lung cancer⁵⁰. In addition, TAMs often further the development of drug resistance within tumors through the release of cytokines and directly stimulate tumor growth by releasing growth factors⁵¹. While it has been reported that TAMs can act as drug depots that sustainably release drug into the surrounding tumor tissue⁵², PEGylated liposomes such as the ones presented herein are known for evasion of the mononuclear phagocyte system, and are unlikely to cause drug depot formation⁵³.

Finally, GEM/DOX was evaluated against equimolar doses of DOX-L and GEM-L in a survival study. Free GEM and pegylated liposomal doxorubicin are commonly used together in clinical trials for metastatic breast cancer⁵⁴, but to our knowledge this is the first report of a co-encapsulated doxorubicin and gemcitabine liposome. In other studies of co-encapsulated liposomes, control single-drug liposomes are administered with same amount of the single drug that is included in the combination liposomes⁵⁵. However, to fully confirm the GEM/DOX combination is superior to either single drug, the control GEM-L and DOX-L liposomes were administered with single drug doses equivalent to the total molar drug encapsulated in the GEM/DOX-L. Half of the mice in the GEM-L treated group to lost over 15% body weight. This was likely caused by toxic accumulation of PEGylated GEM liposomes in tissues, as GEM is unlikely to cause such toxicity in its free form, which suffers from enzyme degradation⁵⁶. Neither DOX-L nor GEM-L extended the overall survival to the extent exhibited by the GEM/DOX-L treated group, indicating that the combination is both more efficacious and safer than each drug alone. This is in contrast to conventional formulations such as Doxil that offer safety benefits but have been ineffective at increasing survival in patients⁵⁷. Upon further research, GEM/DOX liposomes may offer a superior drug combination for breast cancer therapy. We credit this to the combination's elevated Hill coefficient, a parameter that must be considered for a more comprehensive approach to designing drug combinations for clinical use.

Our findings indicate that the Hill coefficient of the dose-response Hill equation can predict efficacy in vivo and should be evaluated along with IC₅₀ and synergy for design of drug combinations. Using these in vitro parameters, we designed and optimized a co-encapsulated chemotherapeutic treatment that considers both the cytotoxicity and the immunogenicity of the drugs. The decrease of M2 macrophages and increase of the M1/M2 ratio enhanced the final GEM/DOX liposomal formulation. Tumor response correlated with increasing Hill coefficient, and not with decreasing IC₅₀. Finally, a two-fold greater median survival rate was found in mice treated with GEM/DOX compared to a higher dose of either liposomal drug alone, as well as disappearance of the primary tumor in 50% of the GEM/DOX treated mice. In summary, this work evaluates several in vitro and in vivo factors that are necessary in the translation of chemotherapeutic nanocarriers that provides a balanced approach that assess the importance of these factors on improving survival.

Materials and Methods

Chemotherapeutic and Liposomes Agents

Doxorubicin and irinotecan were purchased from LC labs (Woburn, Mass.). Gemcitabine was purchased from Oxchem Corporation (Wood Dale, Ill.). 5-fluorouridine-W was a prodrug based on 5-fluorouracil synthesized by Pharmaron (Beijing, China). Lipids such as 1,2-distearoyl-sn-glycero-3-phosphocholine (DSPC) and 1,2-distearoyl-sn-glycero-3-phosphoethanolamine-N-[methoxy(polyethylene glycol (PEG))-2000] (DSPE-mPEG2000) were purchased from Avanti Polar Lipids (Alabaster, Ala.). Cholesterol was purchased from Millipore Sigma (Burlington, Mass.).

Cell Culture, Flow Cytometry, and Tumor Processing Materials

4T1 murine breast cancer cells (ATCC CRL-2539) was purchased from ATCC (Manassas, Va.). The cells were cultured in RPMI-1640 supplemented with 10% fetal bovine serum (FBS) and 1% penicillin/streptomycin, and cellular inhibition assays used 3-(4,5-dimethylthiazol-2-yl)-2,5-diphenyltetrazolium bromide (MTT); all aforementioned materials were purchased from Thermo Fisher Scientific (Waltham, Mass.). Cell culture flasks and 96 well plates were purchased from Corning (Corning, N.Y.). Heparin-coated plasma preparation tubes, Gibco™ Type 1 Collagenase, ACK Lysing Buffer, Invitrogen™ UltraComp eBeads™ Compensation Beads and SYTOX™ Blue Dead Cell Stain were also purchased from Thermo Fisher Scientific (Waltham, Mass.). DNAse I was purchased from Roche (Indianapolis, Ind.). Antibodies (Table 9) were purchased from ThermoFisher Scientific (Waltham, Mass.) and Millipore Sigma (Burlington, Mass.).

Liposome Preparation and Characterization

Liposomes were prepared by the conventional thin-film hydration method²⁸. Briefly, 40 mmol of lipids (56.4% DSPC, 5.3% DSPE-mPEG2000, 38.3% cholesterol) were dissolved in chloroform and dried under vacuum using rotary evaporation. The resulting lipid film was further dried under heating by water bath. The lipid film was resuspended in 1.1 ml of ammonium sulfate buffer (250 mM, pH 5.5). The solution was sonicated and extruded through a 50 nm polycarbonate membrane to form unilamelar liposomes. Both extruder and extruder membranes were purchased from Avestin Inc. (Ottowa, Ontario, Canada).

During liposome preparation, each drug was either passively or actively loaded. Active loading was done by establishing an ammonium sulfate gradient across the liposomal membrane¹⁶. The gradient was created by using size exclusion chromatography (PD-10 Sephadex columns, GE Healthcare) equilibrated with PBS to remove ammonium sulfate salts from the extraliposomal space. After collecting the liposomes from the size exclusion column, drug loading commenced. DOX-only liposomes were made by incubation of 50 μl of 40 mg/ml doxorubicin with 500 μl of blank extruded liposomes at 65° C. for one hour. Free drug was removed using with the size exclusion column with PBS as the mobile phase.

The tryptophan conjugation of 5-FUR served to make the compound weakly basic and allowed loading through the ammonium sulfate pH gradient as well¹². To create 1:1 and 2.5:1 5FURW:DOX liposomes, DOX loading was carried out first with 50 μl of 20 mg/ml DOX in PBS at 65° C. for one hour. A 175 mg/ml solution of 5FURW was adjusted to approximately pH 6. After the one hour DOX incubation, 100 μl of the 175 mg/ml 5FURW solution was added, and kept for a further one hour at 65° C. Then, the liposomes were removed from 65° C. and free drug was removed by size exclusion chromatography.

IRIN was also able to load through the ammonium sulfate gradient mechanism⁵⁸. After extrusion, the liposomes were passed through a size exclusion column equilibrated with milliQ water, to aid in IRIN solubility. 50 μl of 40 mg/ml IRIN was incubated for one hour. DOX loading followed with 50 μl of 40 mg/ml DOX. Free drug was removed using size exclusion chromatography.

However, GEM was unable to be sufficiently encapsulated with active loading methods. We passively loaded gemcitabine by rehydrating the lipid film with 75 mg/ml of GEM in 1.1 ml of ammonium sulfate buffer. During the active loading of DOX (50 μl, 20 mg/ml), 50 μl of 95 mg/ml of GEM was also added to reduce the gemcitabine gradient across the liposomal bilayer. Free drug was removed using PD-10 desalting size exclusion columns from GE Healthcare (Piscataway, N.J.).

The size and surface potential of the liposomes were verified using a Malvern Zetasizer™. Liposomes were diluted 100-fold prior to analysis. To quantify drug loading, liposomes were diluted 10× and disrupted in 1:1 methanol: acetonitrile with 0.05% formic acid. After 30 minutes of sonication, the resulting solution was centrifuged, and the supernatant was removed. The supernatant was further 10× diluted in water with 0.1% formic acid, and drug content was quantified using RP-HPLC with a Zorbx 300Extend™ C18 3.5 μm column (150 mm×4.6 mm) purchased from Agilent (Santa Clara, Calif.). The column was equilibrated with a flow rate of 0.5 ml/min 99% mobile phase A (water with 0.1% trifluoroacetic acid) and 1% mobile phase B (acetonitrile with 0.1% trifluoroacetic acid). Samples were started with 99% mobile phase A and 1% mobile phase B. After 10 minutes, mobile phase B had ramped to 60%. The composition changed back to 99% mobile phase A and 1% mobile phase B at 15 minutes, and was maintained until 20 minutes.

In Vitro Cellular Inhibition and Synergy Quantification

4T1 cells were seeded in 96 well plates at a density of 500 cells/well. Cells were given 24 hours to adhere to the well plate. Afterwards, a series of ten drug or drug combination dilutions prepared in fresh media were administered to the cells, with a starting concentration of 100 μM. The drugs were incubated with the cells for 72 hours before the media was removed and replaced with 0.5 mg/ml of MTT reagent in media fresh media. The MTT reagent was left incubating with the cells for 4 hours, during which living cells metabolized the reagent to form solid formazan crystals. Afterwards, the MTT reagent in media was removed, and the crystallized formazan was dissolved in DMSO. The plate is shaken at 300 rpm for 15 minutes to fully dissolve the formazan crystals, and absorbance was measured at 590 nm via Spectramax i3 plate reader. The fraction of cells inhibited at a certain drug concentration was calculated by:

$${f}_{a} = 1 - \frac{A - {A}_{0}}{{A}_{C} - {A}_{0}}$$

where A is the average absorbance of the treated wells, A₀ is the average absorbance of the blank wells with DMSO, and A_{C} is the average absorbance of the control untreated wells. The IC₅₀ values for each treatment was then determined through fitting of the Hill equation for dose-response in GraphPad™. The Hill equation is given as follows:

$$Y = {( 1 + {( \frac{IC 50}{X} )}^{m} )}^{- 1}$$

with X as the drug concentration and Y as the fractional inhibition, and m is known as the Hill coefficient and is determined according to the fit.

To evaluate drug efficacy against 4T1, drugs were evaluated as single drugs and as combination treatments with DOX. Synergy was quantified using the combination index (CI)¹⁰. The formula for CI is as follows:

$$CI = \frac{{[ {A}_{C} ]}_{50}}{{[ A ]}_{50}} + \frac{{[ {B}_{C} ]}_{50}}{{[ B ]}_{50}}$$

Where [A_{C}]₅₀ and [B_{C}]₅₀ represent the IC₅₀ of drugs A and B when given in combination, and the denominator represents the IC₅₀ of single drugs A and B. CI<1 indicates synergy, while a CI=1 indicates additive effects, and a CI>1 indicates antagonism.

Release and Pharmacokinetic Studies

The release profile of drug from liposomes was studied using Amicon Ultra mini dialysis filters supplied by Millipore Sigma (Burlington, Mass.) at sink conditions⁵⁹. A tenfold dilution of each liposomal formulation in PBS was kept in the mini dialysis filter at 37° C. above a 1.1 ml PBS reservoir. Samples were placed on a plate shaker for constant agitation. At each timepoint (2, 4, 6, 8, and 24 hours), the samples were removed and transferred to fresh PBS reservoirs while the former was analyzed. All PBS reservoirs were analyzed for released drug content using HPLC.

Liposomal pharmacokinetics were also studied to confirm drug circulation in vivo. One 100 μl injection of each liposomal formulation was injected at 0.54 mg/ml DOX into healthy BALB/c mice, resulting in a 3 mg/kg dose. Blood was collected by mandibular puncture at 5 minutes (˜20 μl) and diluted 5-fold with PBS. Blood was also collected by cardiac puncture at 2, 6, and 24 hours after injection and stored in heparin-coated collection tubes from BD (Franklin Lakes, N.J.). Then, the blood was diluted twofold in PBS. 100 μl of the blood/PBS mixture was centrifuged down at 7000 g for 10 minutes to obtain plasma. The plasma was tenfold diluted in 1:1 methanol:acetonitrile organic with 0.05% formic acid for drug extraction. After centrifuging to remove serum proteins, the supernatant was filtered with 0.2 μm syringe filters from Waters (Milford, Mass.) and run using the drug quantification protocol on LC-MS. The mass spectrometer was used to determine if metabolite forms of the drugs were present in the blood.

Tumor Model Development

Murine breast cancer tumors were established by subcutaneous injection of 50 μl containing 10⁵ 4 T1 cells above the 4^{th} abdominal mammary fat pad of BALB/c mice. This method yields uniform breast tumors that resemble human tumors in their metastasis to the lungs and aggressive growth rate^{60,61} Tumor dimensions were measured every other day with calipers, and the tumor volume was calculated using

$$V = \frac{1}{2} ( L ) ( {W}^{2} ) .$$

Once the tumors reached 50 mm³ in volume (˜7 days), liposomal formulations were injected intravenously. For the tumor-associated immune profiling study, the liposomal formulations were injected once at a dose of 3 mg/kg DOX (100 μl of 0.54 mg/ml DOX). Tumors were extracted 10 days after treatment. Afterwards, a full survival study was completed using GEM/DOX-L. GEM/DOX-L was dosed at 3 mg/kg DOX and 1.55 mg/kg GEM. To create equal molar doses of drug in all treatments, the control treatments were 6 mg/kg DOX-L and 3.1 mg/kg GEM-L. The endpoint criteria of the study were a tumor size greater than 1000 mm³ and weight loss exceeding 15% of the starting weight. Animals that developed necrosis in the tumor were also euthanized and excluded from the study. Each treatment group was injected a total of four times with two days between administrations to avoid toxic accumulation of long-circulating liposomes. Surviving mice were monitored for up to 100 days before euthanasia. Tumor growth inhibition percentage was calculated by the following formula,

$$TGI ( \% ) = 100 * ( 1 - \frac{{V}_{{t}_{f}} - {V}_{{t}_{i}}}{{V}_{{c}_{f}} - {V}_{{c}_{i}}} )$$

where V_{tf} and Vₜᵢ represent the final and initial average volumes of the treated tumors, and V_{cf} and V_{ci} represent the final and initial volumes of the control tumors, respectively.

Tumor Processing and Flow Cytometry

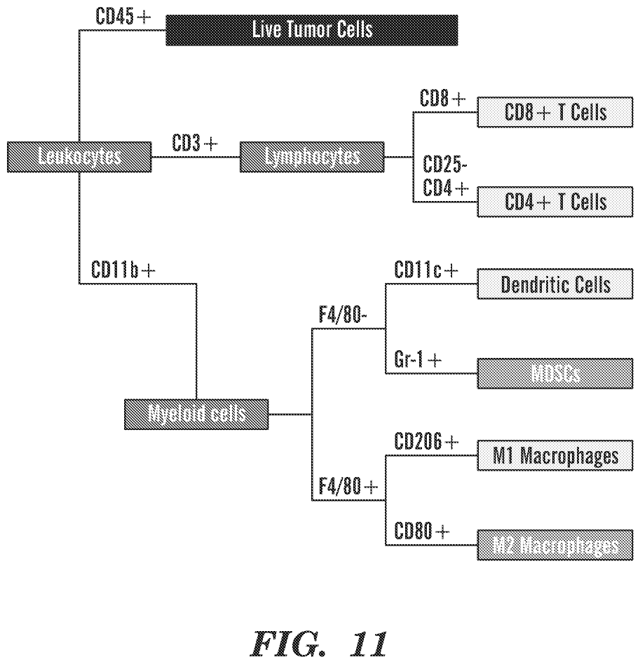

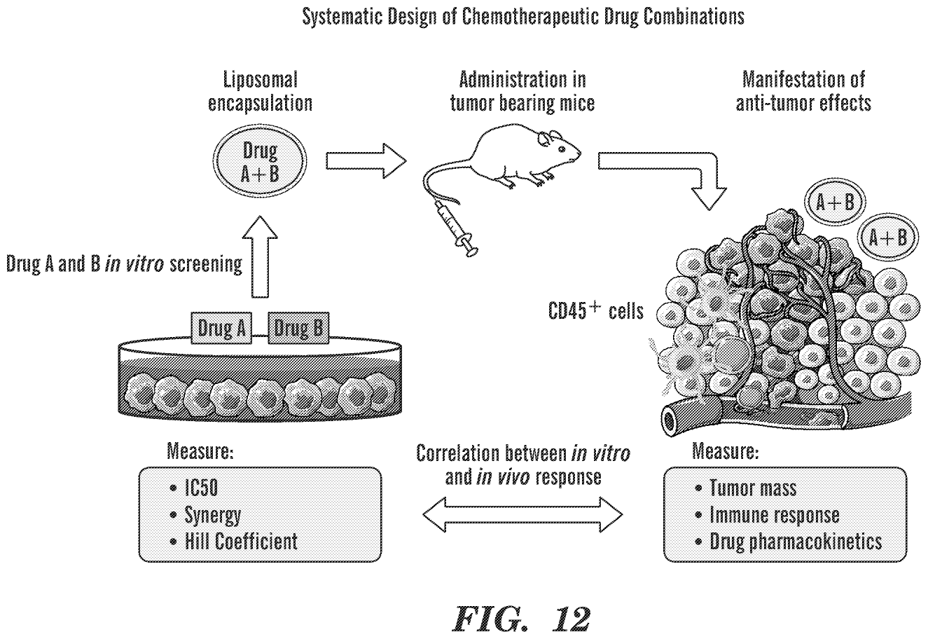

Ten days after the administration of treatment, 4T1 tumors were extracted and weighed. Each tumor was cut into small pieces and enzymatically digested using in 1 ml of Collagenase Type I (5 mg/ml) and DNAse I (20 U/ml) in HBSS buffer at 37° C. for 60 minutes. Afterwards, the cells were passed through 70 μm cell strainers with trituration and then centrifuged and resuspended in ACK red cell lysis buffer for 2 minutes at room temperature. The cells were then resuspended in PBS and 50 U/ml DNAse and adjusted to obtain 10⁶ cells/ml. 100 μl of the cell suspension for each tumor was pelleted and treated with blocking buffer for 30 minutes at room temperature. Blocking buffer was made by supplementing FACS buffer (lx PBS, 3% FBS, 30 μM EDTA) with 5% rat serum, 5% mouse serum, and 1% CD16/32. After washing the cells once with FACS buffer, the tumors from the control group were treated with isotype control antibodies and the tumors from the treatment groups were treated with antibodies specific to immune cell subtypes (FIG. 11). Finally, cells were washed twice more in FACS buffer and subsequently analyzed by flow cytometry (BD LSRII) and all data was analyzed with FCS Express 6™ software (De Novo Software, Glendale, Calif.).

Statistical Analysis

All analysis was performed using GraphPad Prism 5™. The analysis of significance (p<0.05) between treatment tumor volume averages was done either by t-tests or one-way ANOVA test modified with Tukey's multiple comparison test to compare the differences between groups. For analysis of survival data, the study used a Mantel-Cox analysis to test for significance using a p<0.05.

### REFERENCES

1. Kim, Y. S. et al. Oxaliplatin, 5-fluorouracil and Leucovorin (FOLFOX-4) Combination Chemotherapy as a Salvage Treatment in Advanced Gastric Cancer. Cancer Res. Treat. 42, 24 (2010).
2. Sacchi, A. et al. Synergistic antitumor activity of cisplatin, paclitaxel, and gemcitabine with tumor vasculature-targeted tumor necrosis factor-α. Clin. Cancer Res. 12, 175-182 (2006).
3. Mokhtari, R. B. et al. Combination therapy in combating cancer SYSTEMATIC REVIEW: COMBINATION THERAPY IN COMBATING CANCER BACKGROUND. 8, 38022-38043 (2017).
4. Klastersky, J. et al. A four-drug combination chemotherapy with cisplatin, mitomycin, vindesine and 5 fluorouracile: a regimen associated with major toxicity in patients with advanced non-small cell lung cancer. Lung Cancer 11, 373-384 (1994).
5. Conroy, T. et al. FOLFIRINOX or gemcitabine as adjuvant therapy for pancreatic cancer. N Engl. J. Med. 379, 2395-2406 (2018).
6. Qu, C. P. et al. Toxicities of different first-line chemotherapy regimens in the treatment of advanced ovarian cancer: A network meta-analysis. Med. (United States) 96, (2017).
7. Senapati, S., Mahanta, A. K., Kumar, S. & Maiti, P. Controlled drug delivery vehicles for cancer treatment and their performance. Signal Transduct. Target. Ther. 3, 7 (2018).
8. Anselmo, A. C. & Mitragotri, S. Nanoparticles in the clinic. Bioeng. Transl. Med. 1, 10-29 (2016).
9. Hua, S., de Matos, M. B. C., Metselaar, J. M. & Storm, G. Current trends and challenges in the clinical translation of nanoparticulate nanomedicines: Pathways for translational development and commercialization. Front. Pharmacol. 9, 1-14 (2018).
10. Chou, T. C. Drug combination studies and their synergy quantification using the chou-talalay method. Cancer Res. 70, 440-446 (2010).
11. Pusuluri, A. et al. Treating tumors at low drug doses using an aptamer-peptide synergistic drug conjugate. Angew. Chemie Int. Ed. (2018). doi: 10.1002/anie.201812650
12. Camacho, K. M. et al. DAFODIL: A novel liposome-encapsulated synergistic combination of doxorubicin and 5FU for low dose chemotherapy. J. Control. Release 229, 154-162 (2016).
13. Vogus, D. R., Pusuluri, A., Chen, R. & Mitragotri, S. Schedule dependent synergy of gemcitabine and doxorubicin: Improvement of in vitro efficacy and lack of in vitro-in vivo correlation. Bioeng. Transl. Med. 3, 49-57 (2018).
14. Shaikh, I. M. et al. Liposome co-encapsulation of synergistic combination of irinotecan and doxorubicin for the treatment of intraperitoneally grown ovarian tumor xenograft. J. Control. Release 172, 852-861 (2013).
15. Casares, N. et al. Caspase-dependent immunogenicity of doxorubicin-induced tumor cell death. J. Exp. Med. 202, 1691-1701 (2005).
16. Barenholz, Y. Doxil®—The first FDA-approved nano-drug: Lessons learned. J. Control. Release 160, 117-134 (2012).
17. Suk, J. S., Xu, Q., Kim, N., Hanes, J. & Ensign, L. M. PEGylation as a strategy for improving nanoparticle-based drug and gene delivery. Adv. Drug Deliv. Rev. 99, 28-51 (2016).
18. Lancet, J. E. et al. CPX-351 (cytarabine and daunorubicin) Liposome for Injection Versus Conventional Cytarabine Plus Daunorubicin in Older Patients With Newly Diagnosed Secondary Acute Myeloid Leukemia. J. Clin. Oncol. 36, 2684-2692 (2018).
19. Anselmo, A. C. & Mitragotri, S. Nanoparticles in the clinic: An update. Bioeng. Transl. Med. 1, 10-29 (2016).
20. Simpkins, F. et al. Chemoimmunotherapy using pegylated liposomal Doxorubicin and interleukin-18 in recurrent ovarian cancer: a phase I dose-escalation study. Cancer Immunol. Res. 1, 168-178 (2013).
21. Ferrandina, G. et al. Phase III trial of gemcitabine compared with pegylated liposomal doxorubicin in progressive or recurrent ovarian cancer. J. Clin. Oncol. 26, 890-896 (2008).
22. Shoji, T. et al. A phase II study of irinotecan and pegylated liposomal doxorubicin in platinum-resistant recurrent ovarian cancer (Tohoku Gynecologic Cancer Unit 104 study). Cancer Chemother. Pharmacol. 80, 355-361 (2017).
23. Verschraegen, C. F. et al. Phase II study of bevacizumab with liposomal doxorubicin for patients with platinum- and taxane-resistant ovarian cancer. Ann. Oncol. 23, 3104-3110 (2012).
24. Lakkadwala, S. & Singh, J. Dual Functionalized 5-Fluorouracil Liposomes as Highly Efficient Nanomedicine for Glioblastoma Treatment as Assessed in an In Vitro Brain Tumor Model. J. Pharm. Sci. 107, 2902-2913 (2018).
25. Xu, H. et al. Development of high-content gemcitabine PEGylated liposomes and their cytotoxicity on drug-resistant pancreatic tumour cells. Pharm. Res. 31, 2583-2592 (2014).
26. Hong, S. S. et al. Development of paclitaxel-loaded liposomal nanocarrier stabilized by triglyceride incorporation. Int. J. Nanomedicine 11, 4465-4477 (2016).
27. Brunetti, C., Anelli, L., Zagaria, A., Specchia, G. & Albano, F. CPX-351 in acute myeloid leukemia: can a new formulation maximize the efficacy of old compounds? Expert Rev. Hematol. 10, 853-862 (2017).
28. Szoka, F. & Papahadjopoulos, D. Comparative Properties Preparation Of Lipid Vesicles

(Liposomes). Ann Rev. Biophys Bioeng 9, 467-508 (1980).
29. Alexis, F., Pridgen, E., Molnar, L. K. & Farokhzad, 0. C. Factors affecting the clearance and biodistribution of polymeric nanoparticles. Mol. Pharm. 5, 505-515 (2008).
30. Smith, R. J., Lotya, M. & Coleman, J. N. The importance of repulsive potential barriers for the dispersion of graphene using surfactants. New J. Phys. 12, (2010).
31. Zhang, Y., Huo, M., Zhou, J. & Xie, S. PKSolver: An add-in program for pharmacokinetic and pharmacodynamic data analysis in Microsoft Excel. Comput. Methods Programs Biomed. 99, 306-314 (2010).
32. Pulaski, B. A. & Ostrand-Rosenberg, S. Mouse 4T1 Breast Tumor Model. Curr. Protoc. Immunol. (2001). doi:doi:10.1002/0471142735.im2002s39
33. Papahadjopoulos, D. et al. Sterically stabilized liposomes: Improvements in pharmacokinetics and antitumor therapeutic efficacy. Proc. Natl. Acad. Sci. U S. A. 88, 11460-11464 (1991).
34. Lu, J. et al. Breast Cancer Chemo-immunotherapy through Liposomal Delivery of an

Immunogenic Cell Death Stimulus Plus Interference in the IDO-1 Pathway. ACS Nano 12, 11041-11061 (2018).
35. Galluzzi, L., Buqué, A., Kepp, O., Zitvogel, L. & Kroemer, G. Immunological Effects of Conventional Chemotherapy and Targeted Anticancer Agents. Cancer Cell 28, 690-714 (2015).
36. Gebremeskel, S. & Johnston, B. Concepts and mechanisms underlying chemotherapy induced immunogenic cell death: impact on clinical studies and considerations for combined therapies. Oncotarget 6, 41600-41619 (2015).
37. Pfirschke, C. et al. Immunogenic Chemotherapy Sensitizes Tumors to Checkpoint Blockade Therapy. Immunity 44, 343-354 (2016).
38. JY, P. et al. Doxorubicin enhances CD4+ T-cell immune responses by inducing expression of CD40 ligand and 4-1BB. Int. Immunopharmacol. 9, 1530-1539 (2009).
39. Yang, L. & Zhang, Y. Tumor-associated macrophages: from basic research to clinical application. J. Hematol. Oncol. 10, 58 (2017).
40. Federico, C., Morittu, V. M., Britti, D., Trapasso, E. & Cosco, D. Gemcitabine-loaded liposomes: Rationale, potentialities and future perspectives. Int. J. Nanomedicine 7, 5423-5436 (2012).
41. Obeid, M. et al. Calreticulin exposure dictates the immunogenicity of cancer cell death. Nat. Med. 13, 54-61 (2007).
42. Xiao, K. et al. The effect of surface charge on in vivo biodistribution of PEG-oligocholic acid based micellar nanoparticles. Biomaterials 32, 3435-3446 (2011).
43. Weiss, J. The Hill equation revisited: uses and misuses. FASEB J. 11, 835-841 (1997).
44. Ravera, S., Quick, M., Nicola, J. P., Carrasco, N. & Mario Amzel, L. Beyond non-integer Hill coefficients: A novel approach to analyzing binding data, applied to Na+-driven transporters. J. Gen. Physiol. 145, 555-563 (2015).
45. Hafner, M., Niepel, M., Chung, M. & Sorger, P. K. Growth rate inhibition metrics correct for confounders in measuring sensitivity to cancer drugs. Nat. Methods 13, 521-527 (2016).
46. Hagemann, T., Biswas, S. K., Lawrence, T., Sica, A. & Lewis, C. E. Regulation of macrophage function in tumors: The multifaceted role of NF-κB. Blood 113, 3139-3146 (2009).
47. Lan, C. et al. Expression of M2-Polarized Macrophages is Associated with Poor Prognosis for Advanced Epithelial Ovarian Cancer. Technol. Cancer Res. Treat. 12, 259-267 (2013).
48. Zhao, X. et al. Prognostic significance of tumor-associated macrophages in breast cancer: a meta-analysis of the literature. Oncotarget 8, 30576-30586 (2017).
49. Guo, B. et al. M2 tumor-associated macrophages produce interleukin-17 to suppress oxaliplatin-induced apoptosis in hepatocellular carcinoma. Oncotarget 8, 44465-44476 (2017).
50. Rakaee, M. et al. Prognostic Value of Macrophage Phenotypes in Resectable Non-Small Cell Lung Cancer Assessed by Multiplex Immunohistochemistry. Neoplasia (United States) 21, 282-293 (2019).
51. Ireland, L. V. & Mielgo, A. Macrophages and Fibroblasts, Key Players in Cancer Chemoresistance. Front. Cell Dev. Biol. 6, 1-14 (2018).
52. Miller, M. A. et al. Tumour-associated macrophages act as a slow-release reservoir of nano-therapeutic Pt(IV) pro-drug. Nat. Commun. 6, 1-13 (2015).
53. Li, S. D. & Huang, L. Stealth Nanoparticles: High Density but Sheddable PEG is a Key for Tumor Targeting. J Control Release 145, 178-181 (2010).
54. Adamo, V. et al. Pegylated liposomal doxorubicin and gemcitabine in the front-line treatment of recurrent/metastatic breast cancer: A multicentre phase II study. Br. J. Cancer 98, 1916-1921 (2008).
55. Tardi, P. et al. In vivo maintenance of synergistic cytarabine:daunorubicin ratios greatly enhances therapeutic efficacy. Leuk. Res. 33, 129-139 (2009).
56. Ciccolini, J., Serdjebi, C., Peters, G. J. & Giovannetti, E. Pharmacokinetics and pharmacogenetics of Gemcitabine as a mainstay in adult and pediatric oncology: an EORTC-PAMM perspective. Cancer Chemother. Pharmacol. 78, 1-12 (2016).
57. O'Brien, M. E. R. et al. Reduced cardiotoxicity and comparable efficacy in a phase III trial of pegylated liposomal doxorubicin HCl (CAELYX™/Doxil®) versus conventional doxorubicin for first-line treatment of metastatic breast cancer. Ann. Oncol. 15, 440-449 (2004).
58. Chou, T.-H., Chen, S.-C. & Chu, I.-M. Effect of Composition on the stability of liposomal irinotecan prepared by a pH gradient method. J. Biosci. Bioeng. 95, 405-408 (2003).
59. Fugit, K. D. & Anderson, B. D. Dynamic, nonsink method for the simultaneous determination of drug permeability and binding coefficients in liposomes. Mol. Pharm. 11, 1314-1325 (2014).
60. Cho, H. J. et al. Bone marrow-derived, alternatively activated macrophages enhance solid tumor growth and lung metastasis of mammary carcinoma cells in a Balb/C mouse orthotopic model. Breast Cancer Res. 14, R81 (2012).
61. Gao, Z. G., Tian, L., Hu, J., Park, I. S. & Bae, Y. H. Prevention of metastasis in a 4T1 murine breast cancer model by doxorubicin carried by folate conjugated pH sensitive polymeric micelles. J. Control. Release 152, 84-89 (2011).

TABLE 5
Pharmacokinetic parameters

| | | 5FURW-DOX- | | 5FURW-DOX- | | IRIN-DOX- | | GEM-DOX- | |
|---|---|---|---|---|---|---|---|---|---|
| Pharmacokinetic | DOX-L | L_{R=1} | | L_{R=2.5} | | L_{R=1} | | L_{R=1} | |
| parameter^{a} | DOX | 5FURW | DOX | 5FURW | DOX | IRIN | DOX | GEM | DOX |
| Cₘₐₓ (mmol/L) | 0.14 | 0.123 | 0.132 | 0.25 | 0.13 | 0.14 | 0.12 | 0.14 | 0.12 |
| AUC_{0→t} | 2.34 | 1.93 | 2.18 | 3.60 | 1.84 | 1.07 | 2.05 | 2.51 | 2.19 |
| (mmol/L*hr) | | | | | | | | | |
| t_{1/2} (hr) | 16.6 | 14.5 | 16.5 | 11.6 | 12.8 | 4.65 | 20.8 | 14.9 | 21.0 |
| V_{d} (ml) | 0.62 | 0.69 | 0.62 | 0.75 | 0.60 | 0.60 | 0.76 | 0.67 | 0.84 |
| CL (ml/hr) | 0.026 | 0.033 | 0.026 | 0.045 | 0.032 | 0.089 | 0.026 | 0.031 | 0.028 |

^{a}Cₘₐₓ, plasma concentration maximum; AUC, area under the curve representing total drug exposure from t = 0 hr to t = 24 hr; t_{1/2}, half life; V_{d}, volume of distribution; CL, total body clearance.

TABLE 6
Tumor mass statistics using one-way ANOVA
with Tukey's multiple comparison test

| | | | 5FURW/ | 5FURW/ | IRIN/ | GEM/ |
|---|---|---|---|---|---|---|
| | Control | DOX | DOX_{R=2.5} | DOX_{R=1} | DOX_{R=1} | DOX_{R=1} |
| Control | — | | | | | |
| DOX | | — | | | | |
| 5FURW/DOX_{R=2.5} | * | | — | | | |
| 5FURW/DOX_{R=1} | * | | | — | | |
| IRIN/DOX_{R=1} | *** | | | | — | |
| GEM/DOX_{R=1} | **** | * | * | * | | — |

* is p < 0.05,
** is p < 0.01,
*** is p < 0.001,
**** is p < 0.0001

TABLE 7
Median survival

| Treatment | | | |
|---|---|---|---|
| Group | MST¹ (days) | % ILS² | % LTS³ |
| Control | 26 | N/A | 0 |
| DOX-L | 44 | 69 | 17 |
| GEM-L | 32 | 23 | 0 |
| GEM/DOX-L | 88 | 238 | 71 |

¹Median survival time
²Percent increase in life span compared to control group
³Long-term survival, the number of survivors in the group at the end of 60 days

TABLE 8
Comparison of survival curves. Log-rank (Mantel-Cox) Test

| | |
|---|---|
| Chi square | 10.96 |
| df | 3 |
| P value | 0.0120 |
| P value summary | * |

*is p < 0.05

TABLE 9
Antibodies for cell staining flow cytometry

| Antibody | Clone | Host/Isotype | Fluorophore | Supplier |
|---|---|---|---|---|
| F4/80 | BM8.1 | Rat/IgG2a, kappa | PerCP-Cy5.5 | Millipore Sigma |
| CD8a | 53-6.7 | Rat/IgG2a, kappa | PE | ThermoFisher |
| CD11b | M1/70 | Rat/IgG2b, kappa | APC | Scientific |
| CD4 | RM4-5 | Rat/IgG2a, kappa | PE-Cy7 | |
| CD3e | 145-2C11 | Armenian | PerCP-Cy5.5 | |
| | | hamster/IgG | | |
| CD45 | 30-F11 | Rat/IgG2b, kappa | FITC | |
| CD11c | N418 | Armenian | PE | |
| | | hamster/IgG | | |
| Gr-1/Ly-6G | RB6-8C5 | Rat/IgG2b, kappa | PE-Cy7 | |
| CD80 | 16-10A1 | Armenian | PE | |
| | | hamster/IgG | | |
| CD206 | MR6F3 | Rat/IgG2b, kappa | PE-Cy7 | |
| Isotype controls | eB149/10H5 | Rat/IgG2b, kappa | FITC | |
| | eB149/10H5 | Rat/IgG2b, kappa | APC | |
| | eBio299Arm | Armenian | PerCP-Cy5.5 | |
| | | hamster/IgG | | |
| | eBRG1 | Rat/IgG1, kappa | APC | |
| | eBR2a | Rat/IgG2a, kappa | PE-Cy7 | |
| | eBR2a | Rat/IgG2a, kappa | PE | |
| | eBR2a | Rat/IgG2a, kappa | PerCP-Cy5.5 | |
| | eB149/10H5 | Rat/IgG2b, kappa | PE-Cy7 | |
| | eBio299Arm | Armenian | PE | |
| | | hamster/IgG | | |

### Example 2: Gemcitabine and Doxorubicin in Immunostimulatory Monophosphoryl Lipid a Liposomes for Treating Breast Cancer

Cancer therapy is increasingly shifting toward targeting the tumor immune microenvironment and influencing populations of tumor infiltrating lymphocytes. Breast cancer presents a unique challenge as tumors of the triple-negative breast cancer subtype employ a multitude of immunosilencing mechanisms that promote immune evasion and rapid growth. Treatment of breast cancer with chemotherapeutics has been shown to induce underlying immunostimulatory responses that can be further amplified with the addition of immune-modulating agents. Described herein are the effects of combining doxorubicin (DOX) and gemcitabine (GEM), two chemotherapeutics, with monophosphoryl lipid A (MPLA), a clinically used TLR4 adjuvant derived from liposaccharides. MPLA was incorporated into the lipid bilayer of liposomes loaded with a 1:1 molar ratio of DOX and GEM to create an intravenously administered treatment. In vivo studies indicated excellent efficacy of both GEM-DOX liposomes and GEM-DOX-MPLA liposomes against 4T1 tumors. In vitro and in vivo results showed increased dendritic cell expression of CD86 in the presence of liposomes containing chemotherapeutics and MPLA. Despite this, a tumor rechallenge study indicated little effect on tumor growth upon rechallenge, indicating the lack of a long-term immune response. GEM/DOX/MPLA-L displayed remarkable control of the primary tumor growth and is contemplated for the treatment of triple-negative breast cancer.

### Introduction

The engineering of the tumor immune response has rapidly become an integral part of cancer therapies. Treatments such as checkpoint inhibitors have significantly improved patient prognosis in late-stage non-small cell lung cancer¹ and melanoma.² Studies have shown that breast cancer, while traditionally considered immunologically cold,³ may also manifest host antitumor immune responses that may be amplified through use of immunotherapy.^{4,5} However, few clinical trials of checkpoint inhibitor monotherapy in the treatment of triple negative breast cancer have demonstrated substantial efficacy.⁶ The mechanisms by which breast cancer cells escape immune recognition are still not fully recognized, but include recruitment of suppressive immune cells such as regulatory T cells and tumor-associated macrophages, as well as the secretion of immune inhibitory cytokines.⁷ Breast cancer subtypes also express relatively low levels of tumor antigens, which makes recognition difficult for activated cytotoxic T-cells.⁸

The use of immune adjuvants to boost recognition of otherwise poorly immunogenic antigens can potentially improve the immune microenvironment of breast cancer. Clinically approved immune adjuvants include oil/water emulsions, aluminum salts, and agents that activate innate immunity by binding to “Toll”-like receptors (TLRs) that recognize pathogen-associated molecular patterns.⁹ One such adjuvant, monophosphoryl lipid A (MPLA), is a detoxified derivative of lipopolysaccharide (LPS) from Salmonella minnesota R595. MPLA was the first TLR adjuvant approved for clinical use and is currently licensed for use in Ceravix (human papilloma virus-16 and -18 vaccine) and Fendrix (Hepatitis B vaccine).¹⁰MPLA has also been incorporated in liposomes in the malaria vaccine AS01E (or AS01B) and was shown to induce stronger cytotoxic T cell reactions than formulations that had similar composition but smaller particle size.¹¹

Recent work has shown MPLA to be effective in altering the tumor immune environment when used in liposomes containing immune stimulating cytokines.¹² MPLA may also sensitize breast cancer tumors to doxorubicin (DOX) treatment.¹³ However, the effect of MPLA in combination with different drug pairs has not been extensively explored. The immune effects of chemotherapy have long been disregarded, as drug cocktails were administered to the point of patient myelosuppression.¹⁴ Also, human-derived tumor cell lines are typically implanted in immunodeficient mouse models to ensure tumor growth, resulting in the development of most chemotherapy combinations without consideration of immune effects. However, in the past decade focus has shifted to understanding the immune interactions of low-dose chemotherapy with immunotherapy, and the identification of immunogenic chemotherapy combinations that can enhance immune responses.¹⁵⁻¹⁸

We have recently shown very effective tumor control with gemcitabine (GEM) and DOX liposomes in the orthotopic 4T1 murine breast cancer tumor model.¹⁹ GEM and DOX, both chemotherapeutics, were co-loaded into liposomes with lipid content representative of clinically used formulations. DOX has been reported to stimulate immunogenic cell death of tumor cells, prompting immune recognition and activation,²⁰ and GEM has been shown to restrict myeloid-derived suppressor cells while promoting antigen cross-presentation in dendritic cells.²¹ Treatment with the co-loaded liposome in the 4T1 murine breast cancer tumor model produced a moderate response in terms of increased M1/M2 macrophage ratio in the tumor immune infiltrate. Described herein is the incorporation of MPLA into the lipid bilayer of GEM/DOX liposomes and evaluation of the benefit of MPLA addition in terms of immune response and overall efficacy. These results show that GEM/DOX MPLA liposomes induced a strong effect on the growth of primary tumor. MPLA produced short-term immune activation benefits but did not lead to a long-term immune response upon tumor rechallenge. However, the short-term dendritic cell activation, along with the strong effect on the primary tumor, make GEM/DOX/MPLA liposomes suitable for combination with other forms of immunotherapy to better treat triple-negative breast cancer.

Materials and Methods

Liposome Fabrication and Cell Culture Materials

1,2-distearoyl-sn-glycero-3-phosphocholine (DSPC) and 1,2-distearoyl-sn-glycero-3-phosphoethanolamine-N-[methoxy(polyethylene glycol [PEG])-2000] (DSPE-mPEG2000) were purchased from Avanti Polar Lipids (Alabaster, Ala.). Cholesterol was purchased from Millipore Sigma (Burlington, Mass.). MPLA from Salmonella enterica serotype minnesota Re 595 was purchased from Millipore Sigma. Doxorubicin hydrochloride was purchased from LC labs (Woburn, Mass.) and gemcitabine hydrochloride was purchased from Oxchem Corporation (Wood Dale, Ill.).

4T1 murine breast cancer cells (ATCC CRL-2539) and JAWSII immature murine dendritic cells (ATCC CRL-11904) were purchased from ATCC (Manassas, Va.). 4T1 cells were grown in RPMI-1640 supplemented with 10% fetal bovine serum (FBS) and 1% penicillin/streptomycin. JAWSII dendritic cells were grown in alpha-MEM supplemented with 20% FBS, 1% penicillin/streptomycin, 4 mM 1-glutamine, and 5 ng/ml granulocyte-macrophage colony-stimulating factor. Cellular inhibition assays used 3-(4,5-dimethylthiazol-2-yl)-2,5-diphenyltetrazolium bromide (MTT) to quantify cell viability. All materials were purchased from ThermoFisher Scientific (Waltham, Mass.). Cell culture flasks and tissue culture-treated well plates were purchased from Corning (Corning, N.Y.).

Tumor Model and Flow Cytometric Analysis Materials

All animals used were female BALB/c mice (age 50-56 days) purchased from Charles River Laboratories (Wilmington, Mass.). Heparin-coated plasma preparation tubes, Gibco™ Type 1 Collagenase, ACK Lysing Buffer, Invitrogen™ UltraComp eBeads™ Compensation Beads, and SYTOX™ Blue Dead Cell Stain were also purchased from ThermoFisher Scientific. DNAse I was purchased from Roche (Indianapolis, Ind.). Cell staining buffer was purchased from Biolegend (San Diego, Calif.). Round-bottom 96 well plates were purchased from Corning. Antibodies (Table 12) were purchased from ThermoFisher Scientific, Abcam (Cambridge, Mass.), and Biolegend.

GEM/DOX Liposome Fabrication

Liposomes (40 μmol, molar ratio 56.4% DSPC, 5.3% DSPE-mPEG2000, 38.3% cholesterol) were made by the conventional thin-film hydration method. When making MPLA liposomes, 0.5 mg of MPLA was incorporated as well. The lipids were dissolved in chloroform and added to a dry round-bottom flask. The lipids were dried under reduced pressure and heating to produce a thin lipid film. The lipids were then resuspended using 75 mg/ml GEM in 1.1 ml of ammonium sulfate buffer (250 mM, pH 5.5) and hydrated at 70° C. for 30 min, followed by extrusion through a 50 nm polycarbonate membrane to create liposomes of similar size. Then, a pH gradient was created through the removal of extra-liposomal ammonium sulfate salts and unencapsulated GEM by PD-10 size exclusion columns from GE Healthcare (Chicago, Ill.). The pH gradient served to actively load DOX (20 mg/ml, 50 μl) at 65° C. for 30 min. During this step, 100 μl of 95 mg/ml GEM was also added to reduce GEM loss from diffusion. Then, unencapsulated drugs were removed once more by size exclusion chromatography.

Liposome Characterization

Samples were diluted 10-fold in 9:1 methanol: water with 0.05% trifluoroacetic acid. After a brief sonication, MPLA was detected by reverse phase HPLC. A Zorbax 300Extend C18 3.5 μm column (150 mm×4.6 mm) purchased from Agilent (Santa Clara, Calif.) was equilibrated with 0.5 ml/min 40% mobile phase A (1 mM ammonium acetate) and 60% mobile phase B (2-propanol, LC-MS grade). Ten microliters of sample was injected using this solvent composition. The solvent gradient gradually changed to become 100% mobile phase B at 15 min. It was then changed back to 60% mobile phase B and 40% mobile phase A at 20 min and was maintained until the end of the run at 25 min. MPLA eluted at approximately 16 min and was detected by UV absorption at 240 nm.

Liposomal size and zeta potential were measured by dynamic light scattering using a Malvern Zetasizer. Size was obtained from the number distribution. In order to detect drug content, samples were diluted 10-fold in 1:1 methanol: acetonitrile with 0.05% trifluoroacetic acid (n=3). Samples were then sonicated in a water bath for 30 min and centrifuged for 5 min. Sample supernatant was then analyzed for drug concentration by reverse phase HPLC. The Zorbax column used previously in the detection of MPLA was equilibrated with 0.5 ml/min 99% mobile phase A (water with 0.1% trifluoroacetic acid) and 1% mobile phase B (acetonitrile with 0.1% trifluoroacetic acid). Sample (10 μl) was injected at this composition. After injection, the gradient changed to 60% mobile phase B at 10 min. The solvent composition reverted to 1% mobile phase B at 15 min and was maintained until the end of the run at 20 min.

Liposomal release was measured using Amicon Ultra mini dialysis filters purchased from Millipore Sigma. A 10-fold dilution (100 μl) of the liposomes was placed into the mini dialysis filter (n=5), which was installed over a reservoir of PBS. Samples were kept under constant shaking at 37° C. At each timepoint, the PBS reservoirs were replaced to maintain sink conditions. Released drug was quantified using the drug detection HPLC method described previously.

In Vitro Cellular Assays

Cells for antibody staining and flow cytometry studies were plated in 6-well plates in 3 ml of media and allowed to adhere overnight. In single-cell experiments, 9×10⁵ of either JAWSII dendritic cells or 4T1 murine breast cancer cells were plated in 6-well plates. In co-culture experiments, 9×10⁵ cells consisting of a 1:1 ratio of JAWSII dendritic cells and 4T1 murine breast cancer cells were plated. Treatment was administered approximately 24 h after plating. Cells were harvested using 0.5 ml of trypsin and resuspended to establish 10⁶ cells in 100 μl of cell staining buffer. Cells were washed once and incubated at room temperature with 1% CD16/32 in 100 μl of cell staining buffer. After another wash, cells were incubated for 30 min on ice with fluorescently labeled antibodies (Table 12) to distinguish tumor antigens or characteristic markers of immune cell subtypes. Antibody-stained cells were then washed twice before analysis with a BD LSRII flow cytometer.

Tumor Model Development and Treatment

Tumors were developed by injection of 10⁵ 4 T1 cells in PBS above the fourth mammary fat pad in female BALB/c mice. Tumors were monitored every other day through caliper size measurements. When tumors were approximately 50 mm³, which occurred approximately 7 days after injection, tumors were treated with two intravenous injections of liposomal formulations occurring 4 days apart. Tumors were harvested for immune profiling 48 h after the last treatment.

Treatment efficacy was evaluated with the same tumor implantation procedure. Liposomal formulations were administered when tumors were ˜15 mm³. Treatment was administered on day 5, 9, and 16 after tumor inoculation. Tumor volume and mice weight were monitored every other day until the control group tumors reached the endpoint criteria of 1000 mm³, at which point the study was terminated and tumors were extracted for mass measurements. Mice body weight loss greater than 15% was also a criterion for euthanasia.

In performing the tumor rechallenge, tumors were established with the same implantation procedure. When tumors were ˜15 mm³ in size, two injections of liposomal formulations were administered 4 days apart. Tumors were observed for ˜20 days, at which point 10⁵ 4 T1 cells in PBS were injected in the opposite mammary fat pad. Mice were monitored for tumor growth and weight loss.

Tumor Dissociation and Immune Profiling

Two days after the second administration of treatment, 4T1 tumors were extracted and weighed. Each tumor was cut into small pieces and enzymatically digested using Collagenase Type I (5 mg/ml) and DNAse I (50 U/ml) in 5 ml of HBSS buffer at 37° C. for 60 min. Afterwards, the cells were passed through 70 μm cell strainers with trituration and then centrifuged and resuspended in ACK red cell lysis buffer for 2 min at room temperature. The cells were then resuspended in PBS with 50 U/ml DNAse with volume adjusted to obtain 10⁶ cells/ml. One hundred microliters of the cell suspension for each tumor was pelleted and treated with blocking buffer for 30 min at room temperature in a round-bottom 96 cell plate. Blocking buffer was made by supplementing cell staining buffer (lx PBS, 3% FBS, 30 μM EDTA) with 1% CD16/32. After washing the cells once with cell staining buffer, the tumors were treated with cell marker staining antibodies (Table 12). Leukocytes were identified by CD45, and cells of the myeloid lineage were identified by CD11b. Macrophages were identified by CD11b+F4/80+ and further differentiated by CD80 (M1) and CD206 (M2). Dendritic cells were identified by CD11b+CD11c+. Finally, cells were washed twice more in cell staining buffer and subsequently analyzed by a BD LSRII™ flow cytometer manufactured by BD (Franklin Lakes, N.J.) and all data was analyzed with FCS Express 6™ software (De Novo Software, Glendale, Calif.).

Statistical Analysis

Statistical comparison of groups was done using a one-way analysis of variance with Tukey's multiple comparison test and Student's t-test in GraphPad Prism™ v5. Statistical significance was defined as *p<0.05, **p<0.01, ***p<0.001.

Results

Liposome Fabrication

Liposomes were fabricated by the conventional thin-film hydration technique and loaded with an equimolar ratio of GEM and DOX. MPLA was incorporated into the lipid bilayer during creation of the thin lipid film. Liposomes are hereafter referred to by their encapsulated agents, and denoted by -L. Drug loading, evaluated by HPLC, showed equimolar loading of GEM and DOX achieved with active loading of DOX and passive loading of GEM. The liposomal size and zeta potentials were very similar to that of standard DOX liposomes, representative of clinically used Doxil®.²² Additionally, MPLA was quantified as 88.5 μg/ml in the final liposomal formulation. This resulted in a 17.7% encapsulation efficiency and was due to dilution of the liposomes during drug loading and size-exclusion separation processes. The encapsulation efficiency of GEM and DOX remained similar to previously reported values.¹⁹ The size and zeta potential of the formulations remained similar, showing that incorporation of a small amount of MPLA does not significantly change the liposome physical properties (Table 10).

TABLE 10
GEM/DOX MPLA characterization

| | | | | Zeta | |
|---|---|---|---|---|---|
| | Molar ratio | MPLA | Size | potential | |
| | (GEM:DOX) | (μg/ml) | (nm) | (mV) | PDI |
| DOX-L | — | — | 75.5 ± 2.8 | −23.3 ± 1.2 | 0.05 ± 0.02 |
| GEM/DOX-L | 0.8 | — | 72.3 ± 2.3 | −25.6 ± 1.5 | 0.09 ± 0.01 |
| GEM/DOX/ | 1.0 | 88.5 | 72.0 ± 2.1 | −26.3 ± 1.4 | 0.05 ± 0.02 |
| MPLA-L | | | | | |

In Vitro Cellular Activation

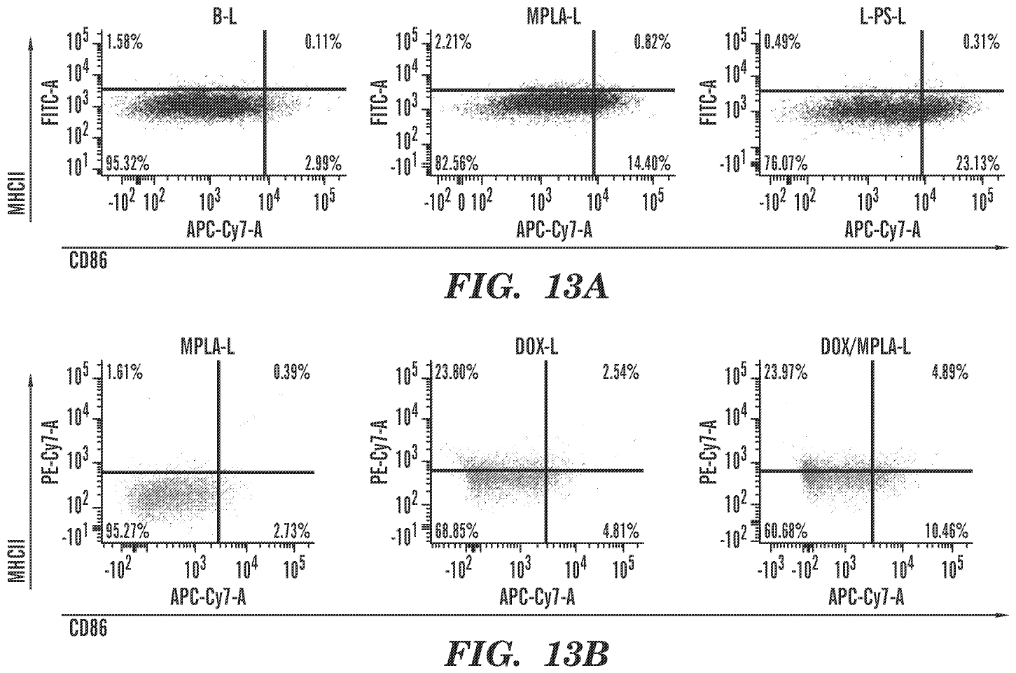

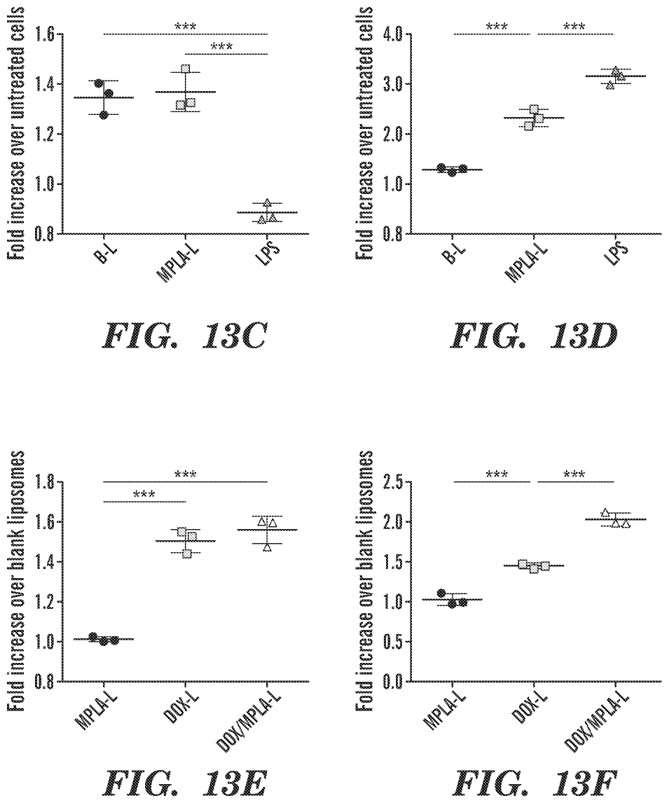

MPLA has been shown to increase dendritic cell activation.^{23,24} Both blank liposomes and liposomes with ˜5 μg/ml MPLA were administered to JAWSII immature murine dendritic cells. 1 μg/ml of liposaccharides (LPS) was used as a positive control for dendritic cell activation. The amount of LPS used was lower than the amount of MPLA because LPS is highly stimulating and a potential cause of decreased cellular viability.²⁵ In JAWSII cells, addition of MPLA-containing liposomes (denoted MPLA-L) did not cause a significant difference in major histocompatibility complex II (MHCII) expression when compared to treatment with an equivalent volume of blank liposomes (denoted B-L) (FIGS. 13A, 13C). However, there was a significant increase in CD86 expression in groups treated with MPLA-L compared to blank liposomes (FIGS. 13A, 13D), indicating greater dendritic cell activation.

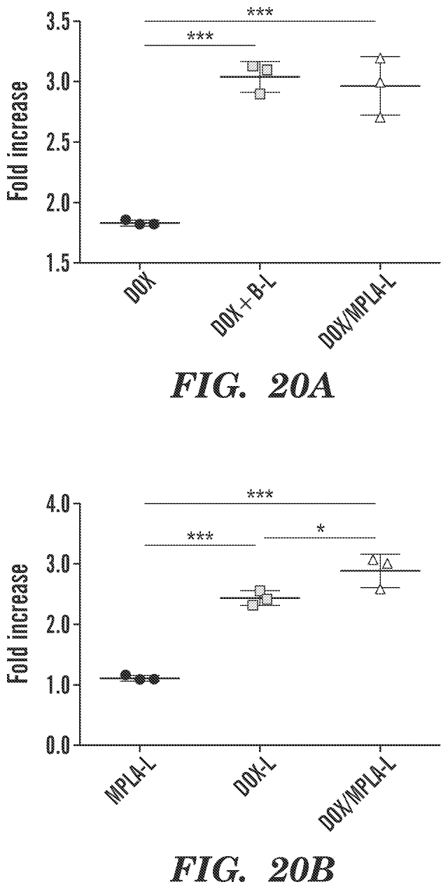

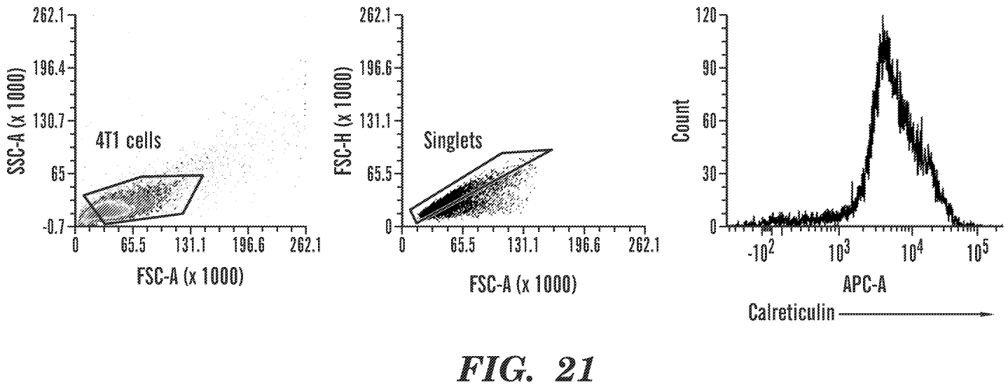

In addition to the immunogenic effects of MPLA, DOX has been shown to increase tumor immunogenic cell death through a variety of mechanisms including the exposure of calreticulin, which stimulates dendritic cell antigen presentation.²⁰ A 1.8-fold increase in calreticulin exposure on 4T1 cells was observed after treatment with 10 μM free DOX compared to untreated controls and increased to approximately threefold upon combination treatment of DOX and liposomes (FIG. 20A). There was no significant difference between the free DOX+blank liposomes and free DOX+MPLA-L, indicating that the inclusion of MPLA does not influence calreticulin exposure. Representative gating for this study is shown in (FIG. 21).

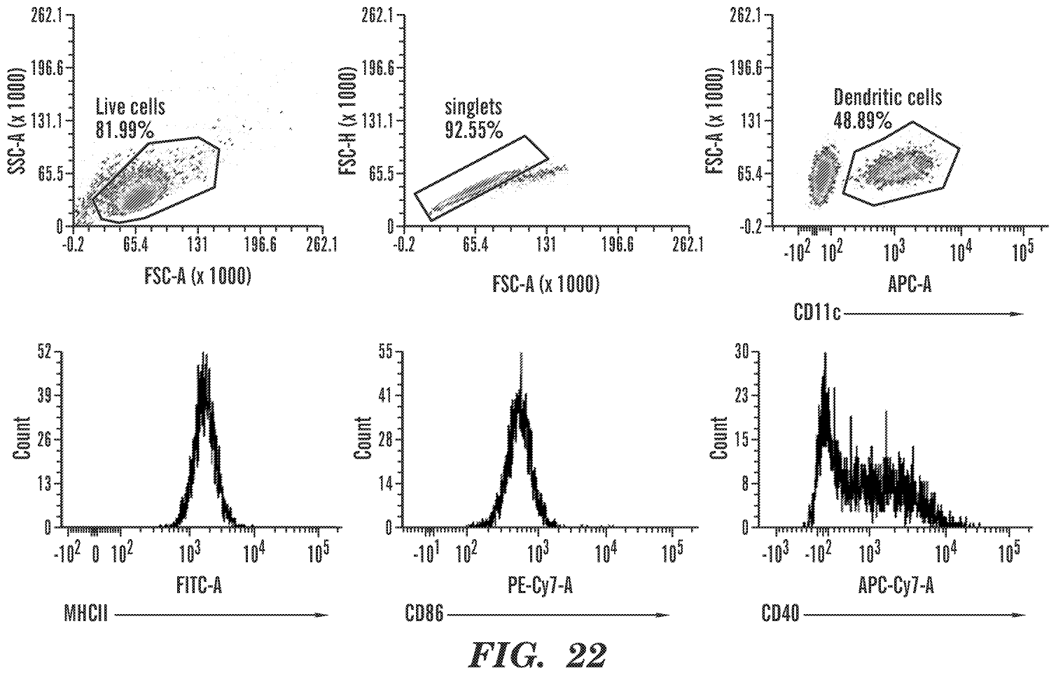

A co-culture of both JAWSII cells and 4T1 cells was developed to study dendritic cell activity in the presence of 4T1 cells, which are shown to undergo immunogenic cell death from exposure to DOX.26 The 1:1 co-culture was treated with MPLA-L, DOX-L, and DOX/MPLA-L. As GEM is not reported to stimulate expression of immunogenic cell death markers, GEM-L and GEM/MPLA-L were not included in this study.¹⁵ The co-culture observed little to no increase in MHCII expression with treatment by MPLA-L alone, possibly due to immunosuppressive signaling produced by 4T1 cells, such as the production of TGF-β and IL-6.²⁷ However, DOX/MPLA-L treatment resulted in a 1.6-fold increase in MHCII expression (FIGS. 13B, 13E) and a twofold increase in CD86 expression (FIGS. 13B, 13F). Another co-stimulatory ligand, CD40, experienced a 2.9-fold increase in expression when treated with DOX/MPLA-L (FIG. 20B). Representative gating of this experiment is reported in FIG. 22.

In Vitro Comparison of Liposomal Toxicity and Release Profile

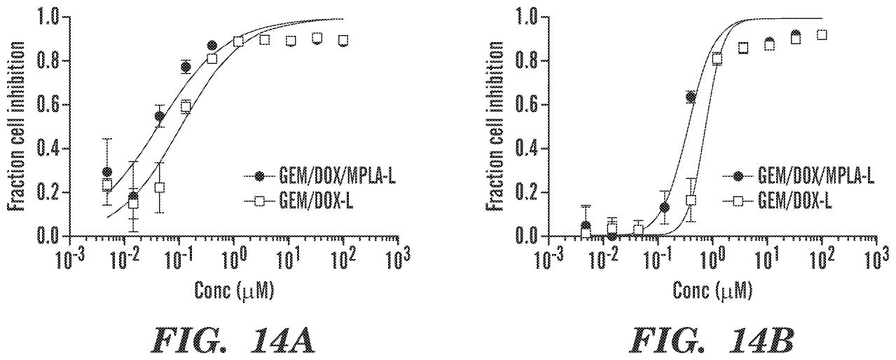

GEM/DOX liposomes containing MPLA (GEM/DOX/MPLA-L) were synthesized and compared to GEM/DOX liposomes without MPLA (GEM/DOX-L) in terms of in vitro cytotoxicity and release profile. The drug combination was previously shown to possess no synergistic effects using the Combination Index on 4T1 cells.¹⁹As MPLA is primarily an immune adjuvant, there was no anticipated effect on 4T1 cells in vitro. Liposomal IC50 and hill coefficient derived from the dose-response Hill equation fitted to cellular viability of 4T1 cells plated at 500 cells/well (FIG. 14A) and 5000 cells/well (FIG. 14B) had no significant differences between the two treatments (Table 11). The IC50 of GEM/DOX-L and GEM/DOX/MPLA-L increased 6.8-fold and 8.8-fold respectively when comparing values from the 500 cell/well and the 5000 cell/well experiments. However, the Hill coefficient of the drug combinations increased to >1 in the 5000 cell/well experiment. The Hill coefficient is an indicator of dose-response curve steepness and can indicate cooperative binding to cell ligands, which may lead to reduction of drug resistance.²⁸ This indicates that while there may be a higher drug concentration threshold to surpass in the case of higher tumor burden, the potency of the drug combination is not lost as high Hill coefficient shows effective tumor control once that threshold is met.

TABLE 11
Dose-response parameters of GEM/DOX-L and GEM/DOX/MPLA-L

| | IC₅₀ (μM) | Hill coefficient |
|---|---|---|
| 500 cells | | |
| GEM/DOX-L | 0.11 ± 0.01 | 0.77 ± 0.06 |
| GEM/DOX/MPLA-L | 0.04 ± 0.01 | 0.69 ± 0.07 |
| 5000 cells | | |
| GEM/DOX-L | 0.75 ± 0.05 | 2.70 ± 0.32 |
| GEM/DOX/MPLA-L | 0.35 ± 0.03 | 1.82 ± 0.26 |

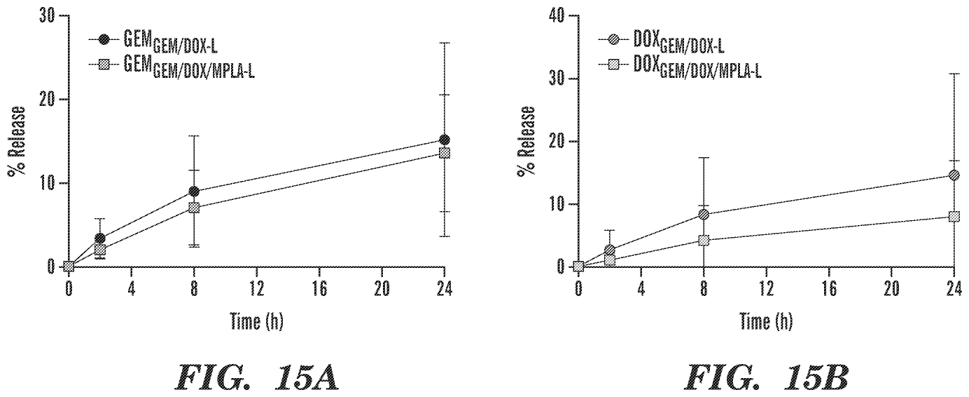

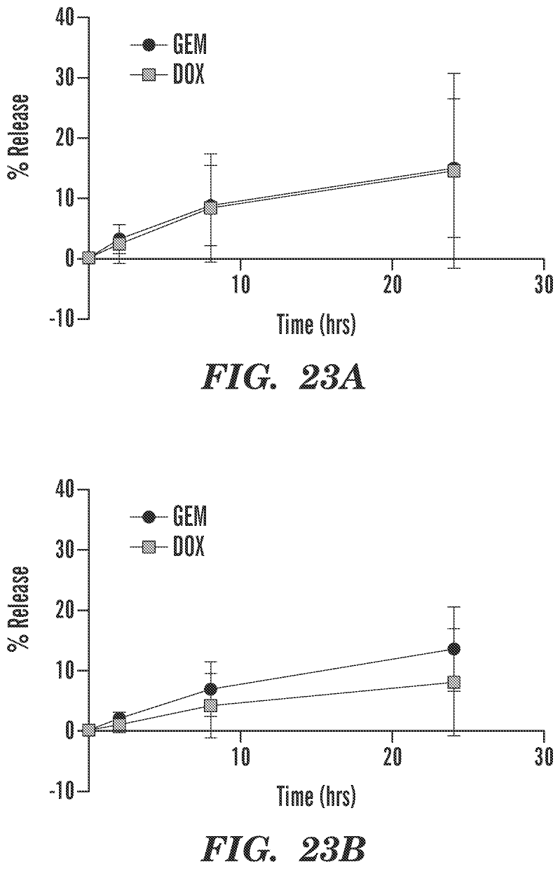

Comparable in vitro toxicity of the liposomal formulations is also an indicator of similar release profiles. The release profile of the formulations into PBS was studied for 24 h at 37° C. under constant shaking to determine if incorporation of MPLA caused significant deviations in drug release. Comparisons between the release of GEM in both GEM/DOX-L and GEM/DOX/MPLA-L showed no significant difference (FIG. 15A) and neither did the release of DOX from both formulations (FIG. 15B). Furthermore, both formulations showed similar release rates of both encapsulated drugs. GEM/DOX-L demonstrated stable encapsulation of drugs with ˜15% of both drugs released at the end of the 24 hr period (FIG. 23A). GEM/DOX/MPLA-L showed similar stable encapsulation, with 14% of GEM released and 8% of DOX released (FIG. 23B). No statistical difference was found between GEM release and DOX release in each formulation. Therefore, MPLA incorporation in the liposomal bilayer did not have a detrimental effect on sustained drug release.

In Vivo Efficacy and Immune Profiling

The liposomal formulations were next evaluated in vivo for immunogenicity and tumor response in the highly aggressive orthotopic 4T1 model. The 4T1 model is also regarded as immunologically cold, making it representative of human breast cancers.²⁹ The liposomal formulations were injected twice intravenously at a dosage of 3 mg/kg DOX and 1.55 mg/kg GEM before tumors were extracted 48 h after the final injection. At that dosage, the GEM/DOX/MPLA-L group delivered a total of 5.7 μg MPLA per injection, which is similar to dosages used in intratumoral injections.^{12, 30}

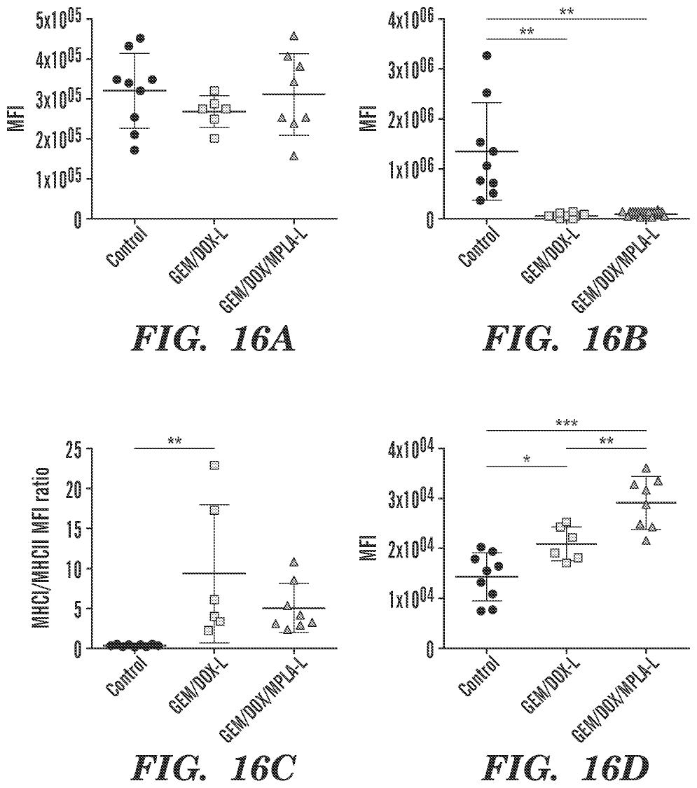

Dendritic cell activation was studied as the fold change in median fluorescence intensity of each treatment group in comparison to the untreated control group. Expression of major histocompatibility complex I (MHC I) (FIG. 16A) and MHC II (FIG. 16B) had no significant difference in expression levels between the treatment groups. MHCII expression was significantly lower in the treatment groups compared to the untreated control group. However, the ratio of MHCI to MHCII expression was significantly elevated in GEM/DOX-L treated mice compared to the control group (FIG. 16C). Antigen presentation by MHC class I molecules has proved essential for recognition by T cell receptors on CD8+ T cells.³¹ Dendritic cell co-stimulatory ligand CD86 was significantly upregulated in the GEM/DOX/MPA-L treatment group (FIG. 16D).

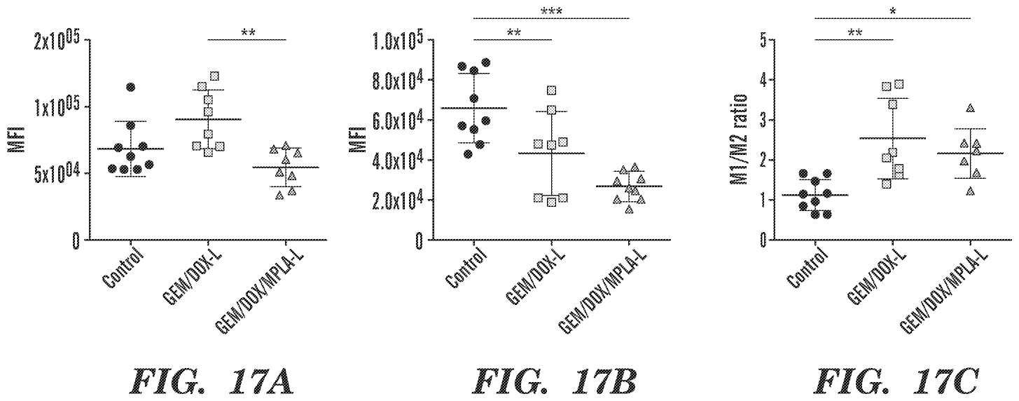

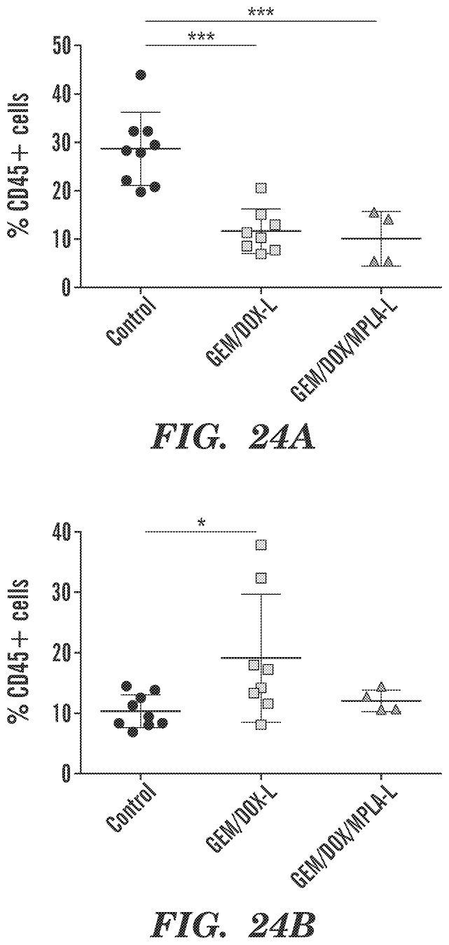

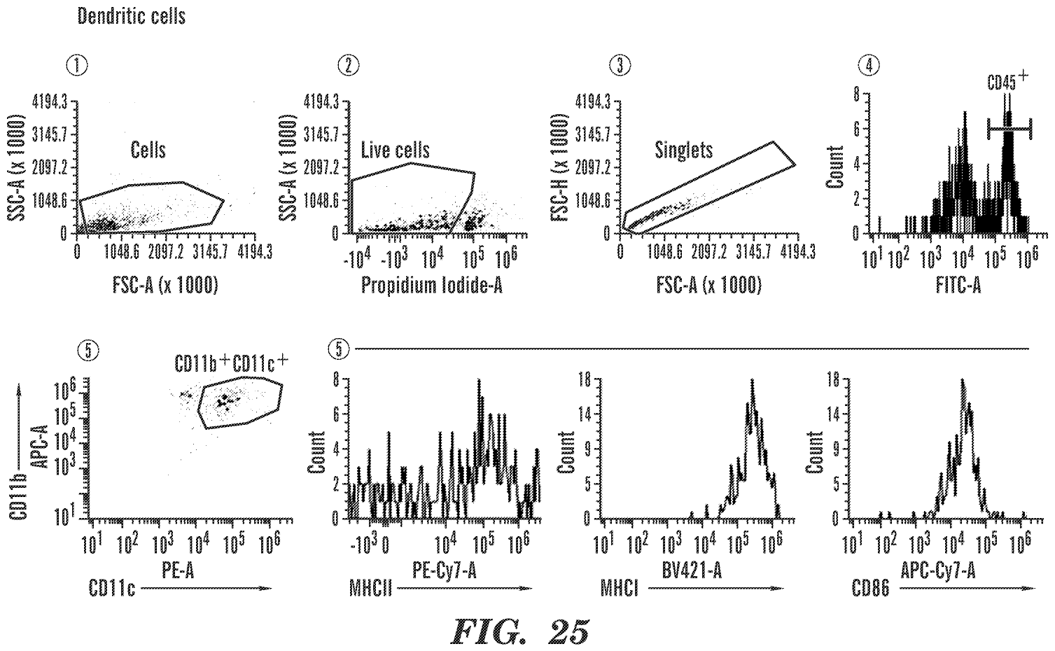

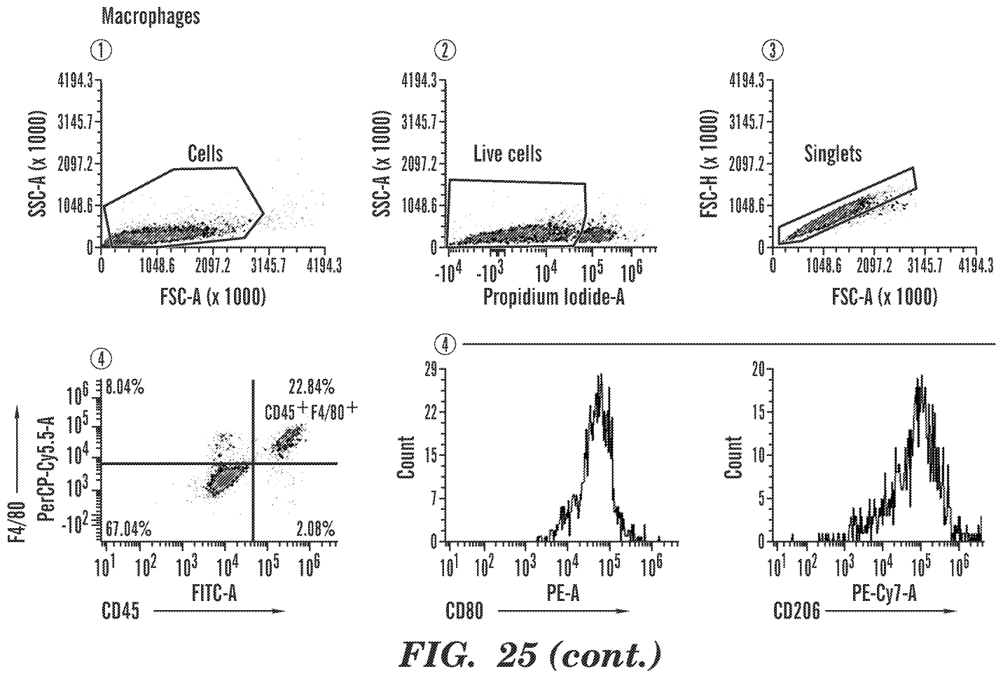

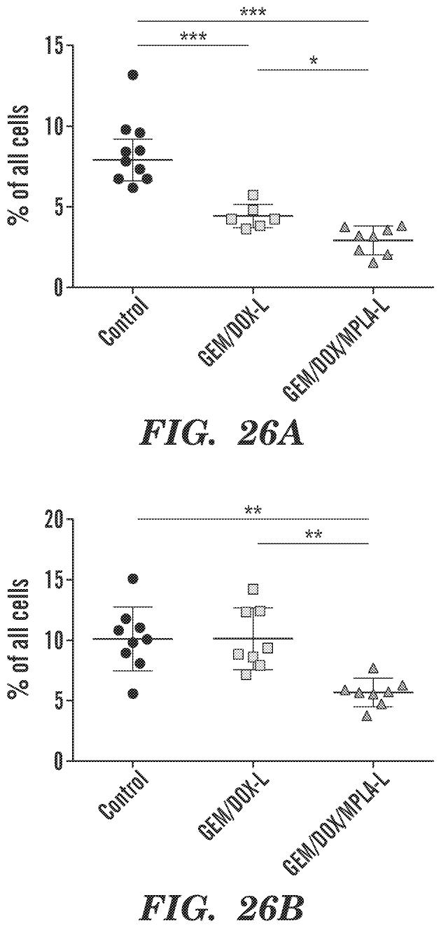

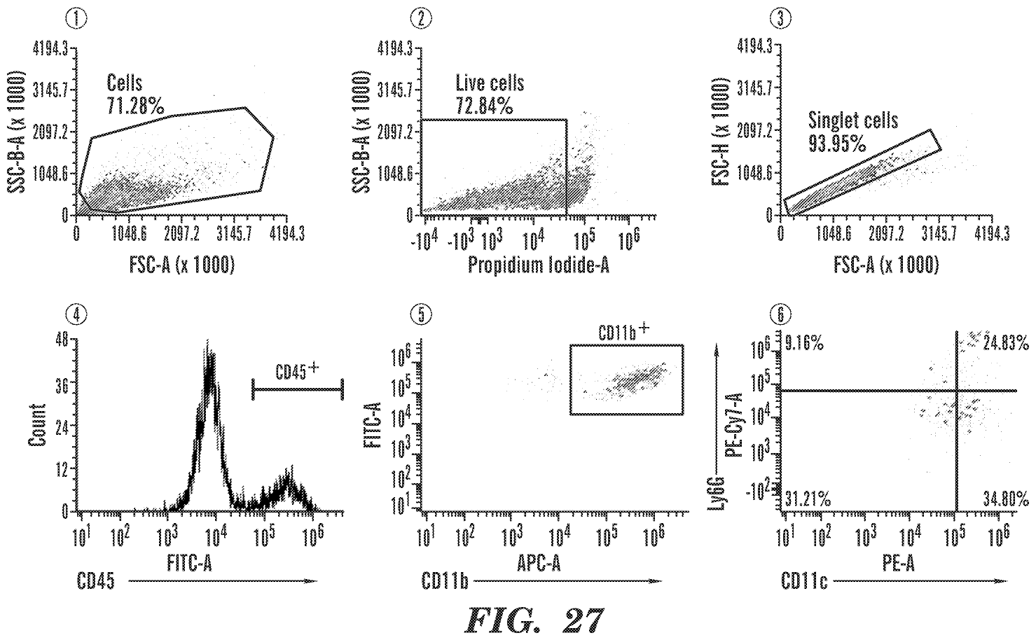

Immune cell populations in the 4T1 tumor environment were quantified by fluorescent antibody staining and analyzed with flow cytometry. The immune effects of the GEM/DOX combination have been shown in previous work to increase macrophage M1/M2 ratio without impacting the adaptive immune response.¹⁹ Similar results were observed in this study. While GEM/DOX-L exhibited increased amounts of CD80+F4/80+M1 macrophages (FIG. 17A) and both treatment groups exhibited decreased CD206+F4/80+M2 macrophages (FIG. 17B), there was ultimately no significant difference between M1/M2 ratio between treatment groups, although both were significantly higher than the control group (FIG. 17C). Negligible differences in CD11c+CD11b+ dendritic cells and Ly6G+CD11b+ myeloid-derived suppressor cells were found between the GEM/DOX-L and GEM/DOX/MPLA-L treatment groups (FIG. 24). Representative gating of in vivo dendritic cells and macrophages are given in FIG. 25, and the cell populations of dendritic cells and macrophages given as a percentage of total cells can be found in FIG. 26. Chemotherapy-treated groups had lower populations of immune cells, although there was no significant difference between the macrophage count of the GEM/DOX-L treated group and the control group. Representative gating of Ly6G+CD1b+ myeloid-derived suppressor cells is given in FIG. 27.

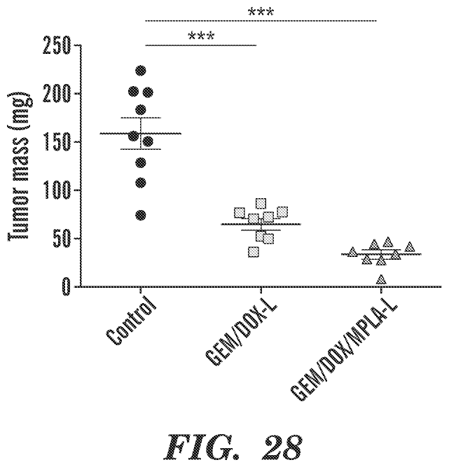

Also, the mass of GEM/DOX-L and GEM/DOX/MPLA-L-treated tumors was significantly less than that of untreated controls, despite undergoing treatment twice with extraction 48 h after the last dosage (FIG. 28).

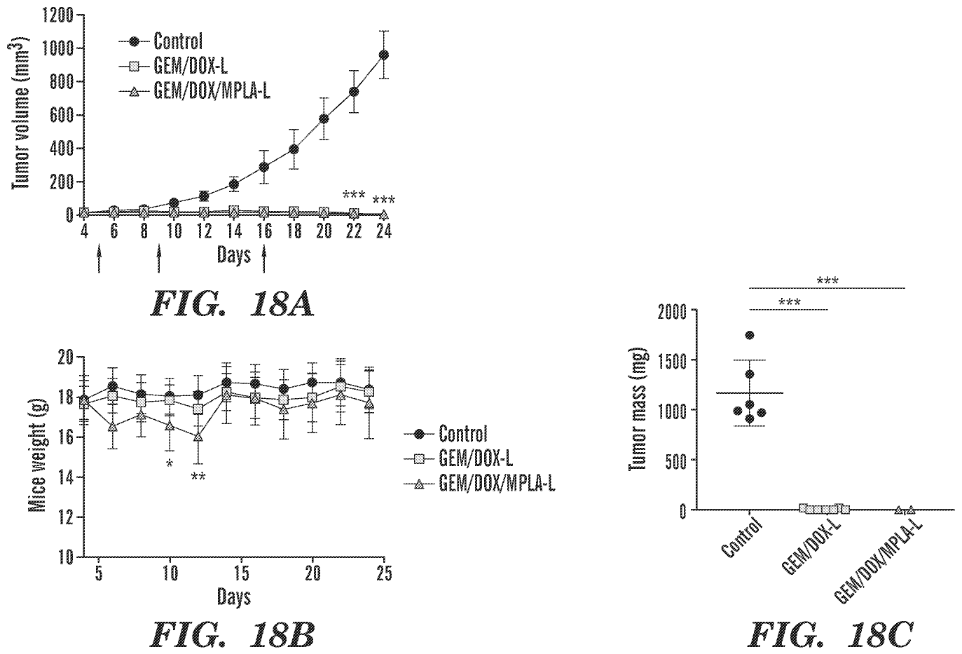

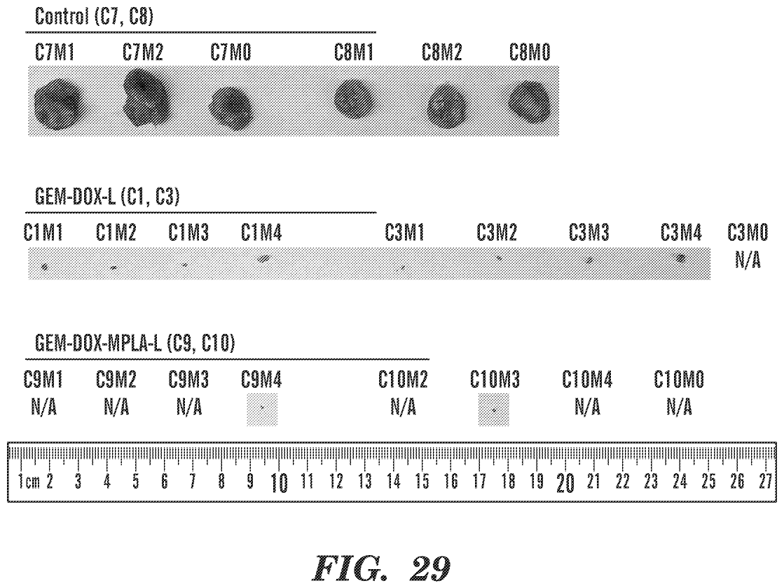

To measure tumor efficacy, treatment was administered when tumors were approximately ˜15 mm³ in size. Treatment comprised of three injections on days 5, 9, and 16 of GEM/DOX-L and GEM/DOX/MPLA-L, both containing 3 mg/kg DOX and 1.55 mg/kg GEM. Mice treated with GEM/DOX/MPLA-L received 5.7 μg MPLA per injection. Tumors were then monitored until the control tumors reached approximately 1000 mm³. The liposomal formulations demonstrated extremely efficient tumor control (FIG. 18A). The 4T1 tumor model is known for aggressive growth and lung metastasis. However, both the GEM/DOX-L and GEM/DOX/MPLA-L formulations managed to limit tumor growth to under 25 mm³. Also, the given dosage of DOX and GEM in co-loaded liposomes was reduced compared to the doses of either drug alone reported in the preclinical literature,^{32, 33} and the dosing schedule allowed for relative stability in mice weight. However, on day 12, mice treated with GEM/DOX/MPLA-L demonstrated significantly more weight loss (*p<0.05) than those treated with the purely chemotherapeutic formulation, which were not significantly different in weight from the control group (FIG. 18B). One of the mice treated with GEM/DOX/MPLA-L was eventually removed from the study due to weight loss greater than 15% of its starting body weight. However, all remaining mice recovered and did not have significantly different weights from the control group by the end of the study. When tumors were extracted at the end of the study on day 27, GEM/DOX/MPLA-L showed no tumor mass in six out of eight mice, whereas GEM/DOX-L led to no detectable tumor mass in only one mouse out of nine. The extracted tumors were weighed, and while both treatment groups had a significantly smaller average mass than the controls, no significant difference could be measured between the treatment groups (FIG. 18C). Tumors after extraction are shown in FIG. 29, and a direct comparison between the tumor masses of GEM/DOX-L and GEM/DOX/MPLA-L is given in FIG. 30.

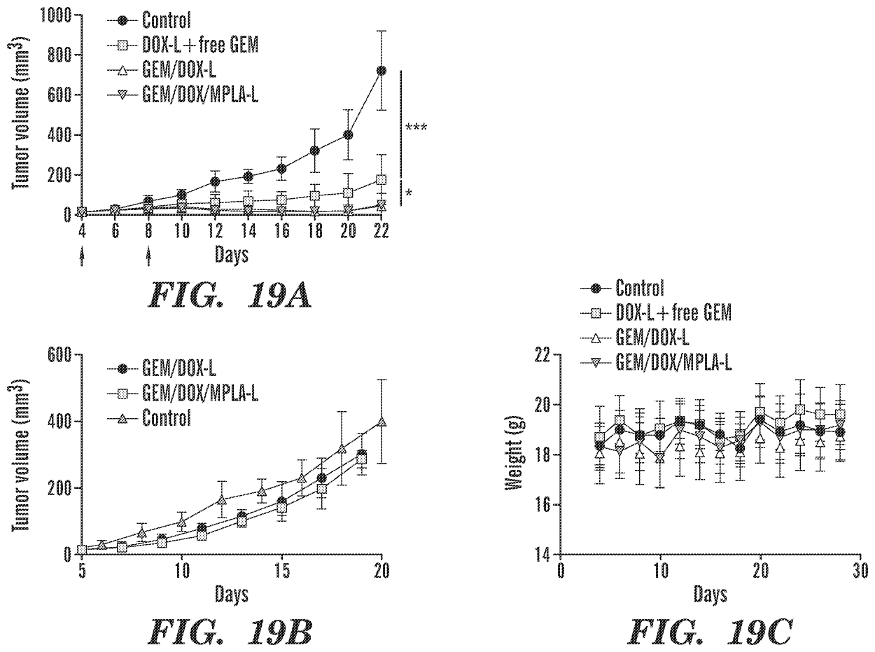

To further investigate and evaluate the relevance of MPLA addition into GEM/DOX liposomes, a tumor rechallenge was conducted in the opposite mammary fat pad using the 4T1 model in BALB/c mice. As before, treatment occurred when tumors were ˜15 mm³ in size. However, one notable difference in this study was that two injections of treatment were given to remain consistent with tumor immune profiling conditions. The MPLA content in this experiment was slightly lower at 4.3 μg per injection. GEM/DOX-L and GEM/DOX/MPLA-L again both showed very similar tumor volume control (FIG. 19A). Upon re-challenge, tumor growth in both groups was similar (FIG. 19B), and there was no weight loss in the MPLA-treated group due to less aggressive dosing (FIG. 19C). The immunogenic cell death of 4T1 cells and enhanced dendritic cell infiltration do not appear to yield long-term immune memory under the current conditions.

Discussion

Effective treatment of breast cancer remains a clinical challenge. This is further compounded by the heterogeneity of breast cancer, which can be generalized by the presence of three receptors: estrogen receptor (ER)-positive, progesterone receptor (PR)-positive, and human epidermal growth factor receptor 2 (HER2)-positive. The lack of all three characteristic receptors defines the triple negative subtype of breast cancer, both the most aggressive and immunosuppressive form of breast cancer.³⁴ The current standard of care for breast cancer includes aggressive chemotherapy regimens, resection, and radiotherapy. However, it is increasingly shown that the tumor microenvironment immune cell infiltrates play a large role in influencing clinical outcome and patient prognosis.^{35, 36} Treatments for breast cancer are rapidly being reconsidered for use with immunotherapy or immunostimulants.

Chemotherapy is traditionally viewed as immunosuppressive, and initially not considered for combination with immunogenic compounds. When dosed at high levels to maximize antitumor cytotoxicity, an unfortunate consequence is the obliteration of immune cell progenitors, leading to severe myelosuppression.

DOX liposomes alone were unable to trigger an adaptive immune response in the highly aggressive 4T1 murine breast cancer tumor model.¹⁹ 4T1 is a form of triple negative breast cancer, which has been shown to have a lower mutational burden than other subtypes of breast cancer. The current approach was to combine MPLA, a potent TLR4 agonist, with a chemotherapeutic combination of DOX and GEM to further amplify the tumor immune response. MPLA has been explored for use in cancer vaccines' but has not been studied extensively in combination with chemotherapy. Here, MPLA was used to enhance the immunogenicity of chemotherapeutics in a novel and translatable dual-loaded liposome with MPLA in the lipid bilayer.

Described herein is a co-loaded DOX, GEM, and MPLA liposomal formulation to ensure controlled drug ratios and consistent MPLA concentration throughout the circulation time of the formulation. The effect of MPLA was confirmed both in vitro and in vivo, and the benefits in tumor efficacy that resulted from this combination were evaluated.

GEM/DOX-L was shown to increase the ratio of CD80+F4/80+(M1) to CD206+F4/80+(M2) macrophages. GEM/DOX-L did not cause significant activation of dendritic cells, which are essential to mounting an anti-tumor immune response. GEM/DOX/MPLA-L treatment did not express significantly higher levels of M1 macrophages than the GEM/DOX-L-treated group. The primary confirmed effect of MPLA in GEM/DOX/MPLA-L was the increase in dendritic cell activation. Dendritic cells are particularly important in mediating the immunogenic cell death process of DOX, as they detect the upregulation of tumor antigens caused by DOX treatment.²⁰ In vitro experiments indicate that DOX combined with MPLA provided higher expression of the tumor antigen calreticulin while MPLA stimulated dendritic cell activation to recognize exposed antigens. The in vivo effect of DOX-initiated immunogenic cell death has been well characterized in the 4T1 tumor model.⁴³

GEM/DOX/MPLA-L was dosed at the same chemotherapeutic drug concentrations as its GEM/DOX-L counterpart (3 mg/kg DOX, 1.55 mg/kg GEM). However, while animals treated with GEM/DOX-L displayed no signs of toxicity, GEM/DOX/MPLA-L appeared to cause more animal weight loss than its GEM/DOX counterpart. Mice injected with GEM/DOX/MPLA-L received 5.7 of MPLA (equating to a 0.3 mg/kg dosage), which falls in the range of MPLA generally used in vaccinations or given intravenously (1-10 μg).⁵⁰ Intravenous administration of MPLA has been given in the range of 0.2-2 mg/kg in C57BL/6 mice.⁵¹

Despite initial immune activation in treated tumors, a tumor rechallenge study with 4T1 cells in the opposite mammary fat pad was not able to produce significant differences in subsequent tumor growth. Other treatments involving immunogenic cell death caused by physical cues such as local photodynamic therapy on 4T1 tumors⁵² and local nanopulse stimulation⁵³ have shown successful reduction of abscopal tumors. Similarly, after vaccination with irradiated CT26 tumor cells treated with a DOX liposome and microbubble complex, rechallenged tumor growth showed reduced tumor volume compared to vaccination with tumor cells treated with control formulations.⁵⁴

GEM/DOX-L also proved to be more effective in reducing tumor size than DOX-L and an equivalent amount of free GEM, which highlights the overall efficacy of the co-encapsulated GEM/DOX combination.

Unique combinations of chemotherapy and immune-modulating agents can influence nonimmunogenic tumor environments to create potential targets for immunotherapies. It is demonstrated herein that the chemotherapeutic combination of GEM and DOX can influence tumor infiltrating lymphocytes when combined with a potent TLR4 agonist, MPLA, in the aggressive 4T1 tumor model. While tumor volume was comparable, GEM/DOX/MPLA-L regressed tumors in six out of eight mice at the time of tumor extraction. However, the rechallenge of tumors in both the GEM/DOX-L and GEM/DOX/MPLA-L treatment groups were unable to suppress growth of newly implanted tumors, indicating the absence of a long-lasting immune memory. The heightened immune response during treatment, however, makes GEM/DOX/MPLA-L an interesting liposomal formulation to pair with immunotherapy.

### REFERENCES

1 Jain P, Jain C, Velcheti V. Role of immune-checkpoint inhibitors in lung cancer. Ther Adv Respir Dis. 2018; 12: 1-13.
2 Buder-Bakhaya K, Hassel J C. Biomarkers for clinical benefit of immune checkpoint inhibitor treatment—a review from the melanoma perspective and beyond. Front Immunol. 2018; 9: 1474.
3 Criscitiello C, Curigliano G. Immunotherapeutics for breast cancer. Curr Opin Oncol. 2013; 25: 602-608.
4 Gatti-Mays M E, Balko J M, Gameiro S R, et al. If we build it they will come: targeting the immune response to breast cancer. NPJ Breast Cancer. 2019; 5: 37.
5 Segovia-Mendoza M, Morales-Montor J. Immune tumor microenvironment in breast cancer and the participation of estrogens and its receptors into cancer physiopathology. Front Immunol. 2019; 10: 1-16.
6 Polk A, Svane I M, Andersson M, Nielsen D. Checkpoint inhibitors in breast cancer—current status. Cancer Treat Rev. 2018; 63: 122-134.
7 Bates J P, Derakhshandeh R, Jones L, Webb T J. Mechanisms of immune evasion in breast cancer. BMC Cancer. 2018; 18: 1-14.
8 Voutsadakis I A. High tumor mutation burden and other immunotherapy response predictors in breast cancers: associations and therapeutic opportunities. Target Oncol. 2020; 15: 127-138.
9 Shi S, Zhu H, Xia X, Liang Z, Ma X, Sun B. Vaccine adjuvants: understanding the structure and mechanism of adjuvanticity. Vaccine. 2019; 37: 3167-3178.
10 Gregg K A, Harberts E, Gardner F M, et al. Rationally designed TLR4 ligands for vaccine adjuvant discovery. MBio. 2017; 8: 1-14.
11 Alving C R, Peachman K K, Rao M, Reed S G. Adjuvants for human vaccines. Curr Opin Immunol. 2012; 24: 310-315.
12 Meraz I M, Savage D J, Segura-Ibarra V, et al. Adjuvant cationic liposomes presenting MPL and IL-12 induce cell death, suppress tumor growth, and alter the cellular phenotype of tumors in a murine model of breast cancer. Mol Pharm. 2014; 11: 3484-3491.
13 Meraz I, Hearnden C H, Liu X, et al. Multivalent presentation of MPL by porous silicon microparticles favors T helper 1 polarization enhancing the anti-tumor efficacy of doxorubicin nanoliposomes. PLoS One. 2014; 9:e94703.
14 Rivera E, Smith R E Jr. Trends in recommendations of myelosuppressive chemotherapy for the treatment of breast cancer: evolution of the National Comprehensive Cancer Network Guidelines and the Cooperative Group studies. Clin Breast Cancer. 2006; 7: 33-41.
15 Pusuluri A, Wu D, Mitragotri S. Immunological consequences of chemotherapy: single drugs, combination therapies and nanoparticle-based treatments. J Control Release. 2019; 305: 130-154.
16 Tesniere A, Schlemmer F, Boige V, et al. Immunogenic death of colon cancer cells treated with oxaliplatin. Oncogene. 2010; 29: 482-491.
17 Pfirschke C, Engblom C, Rickelt S, et al. Immunogenic chemotherapy sensitizes tumors to checkpoint blockade therapy. Immunity. 2016; 44: 343-354.
18 Pusuluri A, Krishnan V, Wu D, Shields C W, Wang L W, Mitragotri S. Role of synergy and immunostimulation in design of chemotherapy combinations: an analysis of doxorubicin and camptothecin. Bioeng Transl Med. 2019; 4: 1-11.
19 Wu D, Pusuluri A, Vogus D, et al. Design principles of drug combinations for chemotherapy. J Control Release. 2020; 323: 36-46.
20 Obeid M, Tesniere A, Ghiringhelli F, et al. Calreticulin exposure dictates the immunogenicity of cancer cell death. Nat Med. 2007; 13: 54-61.
21 Zitvogel L, Apetoh L, Ghiringhelli F, Kroemer G. Immunological aspects of cancer chemotherapy. Nat Rev Immunol. 2008; 8: 59-73.
22 a Gabizon H S, Barenholz Y. Pharmacokinetics of pegylated liposomal doxorubicin: review of animal and human studies. ClinPharmacokinet. 2003; 42: 419-436.
23 Ismaili J, Rennesson J, Aksoy E, et al. Monophosphoryl lipid A activates both human dendritic cells and T cells. J Immunol. 2002; 168: 926-932.
24 Maji M, Mazumder S, Bhattacharya S, et al. Based antigen delivery system efficiently facilitates MHC class-I antigen presentation in dendritic cells to stimulate CD8+ T cells. Sci Rep.
25 Martin I, Caban-Hernandez K, Figureueroa-Santiago O, Espino A M. Fasciola hepatica fatty acid binding protein inhibits TLR4 activation and suppresses the inflammatory cytokines induced by lipopolysaccharide in vitro and in vivo. J Immunol. 2015; 194: 3924-3936.
26 Gao J, Deng F, Jia W. Inhibition of indoleamine 2,3-dioxygenase enhances the therapeutic efficacy of immunogenic chemotherapeutics in breast cancer. J Breast Cancer. 2019; 22: 196-209.
27 Hsieh C C, Wang C H. Aspirin disrupts the crosstalk of angiogenic and inflammatory cytokines between 4t1 breast cancer cells and macrophages. Mediators Inflamm. 2018; 2018: 1-12.
28 Prinz H. Hill coefficients, dose-response curves and allosteric mechanisms. J Chem Biol. 2010; 3: 37-44.
29 Lechner M G, Karimi S S, Barry-holson K, et al. Immunogenicity of murine solid tumor models as a defining feature of in vivo behavior and response to immunotherapy. J Immunother. 2013; 36: 477-489.
30 Van De Voort T J, Felder M A R, Yang R K, Sondel P M, Rakhmilevich A L. Intratumoral delivery of low doses of anti-CD40 mAb combined with monophosphoryl lipid A induces local and systemic antitumor effects in immunocompetent and T cell-deficient mice. J Immunother. 2013; 36: 29-40.
31 Fehres C M, Unger W W J, Garcia-Vallejo J J, van Kooyk Y. Understanding the biology of antigen cross-presentation for the design of vaccines against cancer. Front Immunol. 2014; 5: 1-10.
32 Papahadjopoulos D, Allen T M, Gabizon A, et al. Sterically stabilized liposomes: improvements in pharmacokinetics and antitumor therapeutic efficacy. Proc Natl Acad Sci USA. 1991; 88: 11460-11464.
33 Federico C, Morittu V M, Britti D, Trapasso E, Cosco D. Gemcitabine-loaded liposomes: rationale, potentialities and future perspectives. Int J Nanomed. 2012; 7: 5423-5436.
34 Stagg J, Allard B. Immunotherapeutic approaches in triple-negative breast cancer: latest research and clinical prospects. Ther Adv Med Oncol. 2013; 5: 169-181.
35 Bates G J, Fox S B, Han C, et al. Quantification of regulatory T cells enables the identification of high-risk breast cancer patients and those at risk of late relapse. J Clin Oncol. 2006; 24: 5373-5380.
36 Wang M, Zhang C, Song Y, et al. Mechanism of immune evasion in breast cancer. Onco Targets Ther. 2017; 10: 1561-1573.
37 Hodge J W, Garnett C T, Farsaci B, et al. Chemotherapy-induced immunogenic modulation of tumor cells enhances killing by cytotoxic T lymphocytes and is distinct from immunogenic cell death. Int J Cancer. 2013; 133: 624-636.
38 Wu J, Waxman D J. Metronomic cyclophosphamide eradicates large implanted GL261 gliomas by activating antitumor Cd8+ T-cell responses and immune memory. Onco Targets Ther. 2015; 4: 1-13.
39 Bracci L, Schiavoni G, Sistigu A, Belardelli F. Immune-based mechanisms of cytotoxic chemotherapy: implications for the design of novel and rationale-based combined treatments against cancer. Cell Death Differ. 2014; 21: 15-25.
40 Sakamoto J, Morita S, Oba K, et al. Efficacy of adjuvant immunochemotherapy with polysaccharide K for patients with curatively resected colorectal cancer: a meta-analysis of centrally randomized controlled clinical trials. Cancer Immunol Immunother. 2006; 55: 404-411.
41 Pastore A, Jurinovic V, Kridel R, et al. Integration of gene mutations in risk prognostication for patients receiving first-line immunochemotherapy for follicular lymphoma: a retrospective analysis of a prospective clinical trial and validation in a population-based registry. Lancet Oncol. 2015; 16: 1111-1122.
42 Yan Y, Kumar A B, Finnes H, et al. Combining immune checkpoint inhibitors with conventional cancer therapy. Front Immunol. 2018; 9: 1739.
43 Lu J, Liu X, Liao Y P, et al. Breast cancer chemo-immunotherapy through liposomal delivery of an immunogenic cell death stimulus plus interference in the IDO-1 pathway. ACS Nano. 2018; 12: 11041-11061.
44 Zhong Z, Wei X, Qi B, et al. A novel liposomal vaccine improves humoral immunity and prevents tumor pulmonary metastasis in mice. Int J Pharm. 2010; 399: 156-162.
45 Julka P K, Chacko R T, Nag S, et al. A phase 2 study of sequential neoadjuvant chemotherapy with gemcitabine and doxorubicin followed by gemcitabine and cisplatin in patients with large or locally advanced operable breast cancer: results from long-term follow-up. Breast Cancer. 2013; 20: 357-362.
46 Jacquin J-P, Chargari C, Thorin J, et al. Phase II trial of pegylated liposomal doxorubicin in combination with gemcitabine in metastatic breast cancer patients. Am J Clin Oncol. 2012; 31: 18-21.
47 Zhang Y, Bush X, Yan B, Chen J A. Gemcitabine nanoparticles promote antitumor immunity against melanoma. Biomaterials. 2019; 189: 48-59.
48 Le H K, Graham L, Cha E, Morales J K, Manjili M H, Bear H D. Gemcitabine directly inhibits myeloid derived suppressor cells in BALB/c mice bearing 4T1 mammary carcinoma and augments expansion of T cells from tumor-bearing mice. Int Immunopharmacol. 2009; 9: 900-909.
49 Vincent J, Mignot G, Chalmin F, et al. 5-Fluorouracil selectively kills tumor-associated myeloid-derived suppressor cells resulting in enhanced T cell-dependent antitumor immunity. Cancer Res. 2010; 70: 3052-3061.
50 Ko E J, Lee Y T, Lee Y, Kim K H, Kang S M. Distinct effects of monophosphoryl lipid A, oligodeoxynucleotide CpG, and combination adjuvants on modulating innate and adaptive immune responses to influenza vaccination. Immune Netw. 2017; 17: 326-342.
51 Jie C, Sun B, Zhao X, et al. Monophosphoryl lipid A induces bone marrow precursor cells to differentiate into myeloid-derived suppressor cells. Mol Med Rep. 2013; 8: 1074-1078.
52 Chen Z, Liu L, Liang R, et al. Bioinspired hybrid protein oxygen nanocarrier amplified photodynamic therapy for eliciting anti-tumor immunity and abscopal effect. ACS Nano. 2018; 12: 8633-8645.
53 Beebe S J, Lassiter B P, Guo S. Nanopulse stimulation (NPS) induces tumor ablation and immunity in orthotopic 4T1 mouse breast cancer: a review. Cancers (Basel). 2018; 10: 1-11.
54 Huang F Y, Lei J, Sun Y, et al. Induction of enhanced immunogenic cell death through ultrasound-controlled release of doxorubicin by liposome-microbubble complexes. Onco Targets Ther. 2018; 7: 1-16.

Supplemental Materials

TABLE 12
Antibodies used in staining cell markers in flow cytometry analysis

| Antibody | Clone | Host | Fluorophore | Supplier |
|---|---|---|---|---|
| CD45 | 30-F11 | Rat | FITC | Thermo |
| CD11b | M1/70 | Rat | APC | Fisher |
| CD11c | N418 | Armenian | PE | |
| | | hamster | | |
| CD3e | 145-2C11 | Armenian | PerCP-Cy5.5 | |
| | | hamster | | |
| CD4 | GK1.5 | Rat | PE-Cyanine7 | |
| CD8a | 53-6.7 | Rat | PE | |
| F4/80 | BM8 | Rat | PerCP-Cyanine5.5 | |
| CD206 | MMR | Rat | PE-Cyanine7 | |
| CD80 | B7-1 | Armenian | PE | |
| | | hamster | | |
| Ly-6G | RB6-8C5 | Rat | PE-Cyanine7 | |
| Calreticulin | EPR3924 | Rabbit | Alexa Fluor 647 | Abcam |
| MHC II | M5/114.15.2 | Rat | PE-Cy7 | Biolegend |
| MHC I | SF1-1.1 | Mouse | BV421 | |
| CD86 | GL-1 | Rat | APC-Cyanine7 | |

### Example 3

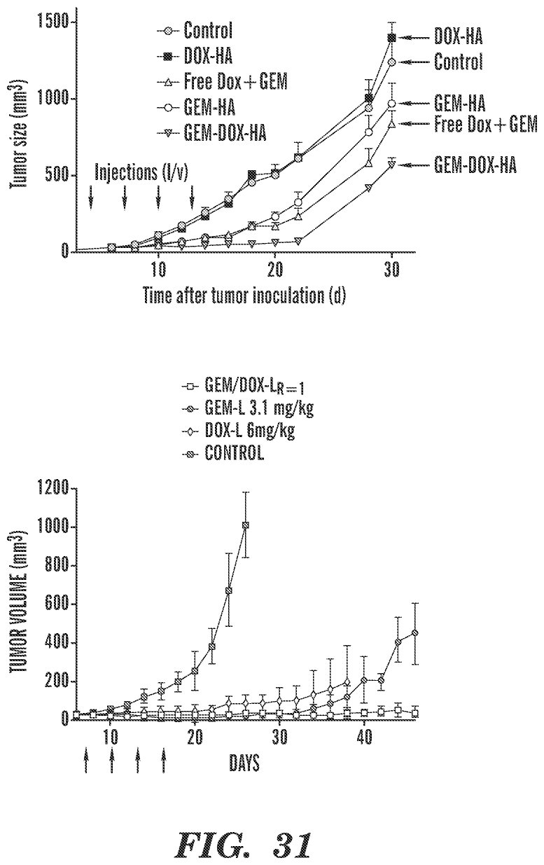

While the drug combination of GEM+DOX provides synergistic benefits when provided as a polymer drug conjugate, the synergistic benefit is strikingly increased when the same drugs are provided in a liposome. See, e.g., FIG. 31, which contrasts polymer drug conjugate data from J Control Release. 2017 Dec. 10; 267:191-202 with newly obtained liposome data. Accordingly, a GEM+DOX liposome combination provides surprising results relative to a GEM+DOX polymer conjugate.

1. (canceled)

2. A method of treating cancer in a subject in need thereof with a drug combination, the method comprising:
a. contacting cancer cells in vitro with at least two different candidate combinations of the candidate drugs;
b. measuring the in vitro dose response of the cancer cells to each candidate combination of step a;
c. calculating the Hill coefficient from the dose response measured in step b;
d. administering the combination with the largest Hill coefficient to the subject.

3.-21. (canceled)

22. A method of treating cancer in a subject in need thereof, the method comprising administering to the subject a liposomal composition comprising individual liposomes each comprising both gemcitabine and doxorubicin.

23. The method of claim 22, wherein the gemcitabine and doxorubicin are present at a molar ratio of from 0.5:1 to 2:1.

24. The method of claim 22, wherein the gemcitabine and doxorubicin are present at a molar ratio of about 1:1.

25. The method of claim 22, wherein the composition further comprises one or more adjuvants.

26. The method of claim 25, wherein the one or more adjuvants comprise one or more TLR4 adjuvants.

27. The method of claim 26, wherein the TLR4 adjuvant is monophosphoryl lipid A (MPLA).

28. The method of claim 22, wherein the cancer is breast cancer.

29. A liposomal composition comprising individual liposomes each comprising both gemcitabine and doxorubicin.

30. The composition of claim 29, wherein the gemcitabine and doxorubicin are present at a molar ratio of from 0.5:1 to 2:1.

31. The composition of claim 29, wherein the gemcitabine and doxorubicin are present at a molar ratio of about 1:1.

32. The composition of claim 29, wherein the composition further comprises one or more adjuvants.

33. The composition of claim 32, wherein the one or more adjuvants comprise one or more TLR4 adjuvants.

34. The composition of claim 33, wherein the TLR4 adjuvant is monophosphoryl lipid A (MPLA).

35.-36. (canceled)
